(19) United States
(12) Patent Application Publication
(10) Pub. No.: US 2024/0200106 A1
(43) Pub. Date: Jun. 20, 2024

(54) LIPID NANOPARTICLES COMPOSITIONS

(71) Applicant: Intellia Thrapeutics, Inc., Cambridge, MA (US)

(72) Inventors: Archana Swami, Cambridge, MA (US); Vishal Rakshe, Cambridge, MA (US); Aaron Prodeus, Cambridge, MA (US); Micah Maetani, Cambridge, MA (US); Rubina Giare Parmar, Cambridge, MA (US)

(21) Appl. No.: 18/287,229

(22) PCT Filed: Apr. 15, 2022
(86) PCT No.: PCT/US22/25074

§ 371 Date: Oct. 17, 2023

Related U.S. Application Data
(60) Provisional application No. 63/316,575, filed on Mar. 4, 2022.
(60) Provisional application No. 63/274,171, filed on Nov. 1, 2021.
(60) Provisional application No. 63/176,228, filed on Apr. 17, 2021.

(51) Int. Cl. C12N 15/90; B82Y 5/00; C12N 9/22; C12N 15/11; C12N 15/88

(52) U.S. Cl. CPC C12N 15/907; C12N 9/22; C12N 15/11; C12N 15/88; B82Y 5/00; C12N 2310/20

(57) ABSTRACT

The disclosure provides lipid nanoparticle (LNP) compositions of ionizable lipids, helper lipids, neutral lipids, and PEG lipids useful for the delivery of biologically active agents, for example delivering biologically active agents to cells to prepare engineered cells. The LNP compositions disclosed herein are useful in methods of gene editing and methods of delivering a biologically active agent and methods of modifying or cleaving DNA.

### CROSS-REFERENCE TO RELATED APPLICATIONS

This application claims the benefit of priority to U.S. Provisional Patent Application No. 63/176,228, filed Apr. 17, 2021; U.S. Provisional Patent Application No. 63/274,171, filed Nov. 1, 2021; and United States Provisional Patent Application No. 63/316,575, filed Mar. 4, 2022, the entire contents of each of which are incorporated herein by reference.

### BACKGROUND

Lipid nanoparticles formulated with ionizable lipids can serve as cargo vehicles for delivery of biologically active agents, in particular polynucleotides, such as RNAs, including mRNAs and guide RNAs into cells. The LNP compositions containing ionizable lipids can facilitate delivery of oligonucleotide agents across cell membranes, and can be used to introduce components and compositions for gene editing into living cells. Biologically active agents that are particularly difficult to deliver to cells include proteins, nucleic acid-based drugs, and derivatives thereof, particularly drugs that include relatively large oligonucleotides, such as mRNA. Compositions for delivery of promising gene editing technologies into cells, such as for delivery of CRISPR/Cas9 system components, are of particular interest (e.g., mRNA encoding a nuclease and associated guide RNA (gRNA)).

There is a need for compositions for improved delivery of nucleic acids, such as RNAs, in vivo and in vitro. As an example, compositions for delivery of the components of CRISPR/Cas to a eukaryotic cell, such as a human cell, are needed. In particular, compositions for delivering mRNA encoding the CRISPR protein component, and for delivering CRISPR gRNAs are of particular interest. Compositions with useful properties for in vitro and in vivo delivery that can stabilize and deliver RNA components, are also of particular interest.

### BRIEF SUMMARY

The present disclosure provides lipid compositions (e.g., nanoparticle (LNP) compositions). Such lipid compositions may have properties advantageous for delivery of biological agents including, e.g., nucleic acid cargo, such as CRISPR/Cas gene editing components, to cells.

In some embodiments, the LNP composition comprises:
a biologically active agent; and
a lipid component, wherein the lipid component comprises:
a) an ionizable lipid in an amount from about 25-50 mol % of the lipid component;
b) a neutral lipid in an amount from about 7-25 mol % of the lipid component;
c) a helper lipid in an amount from about 39-65 mol % of the lipid component; and
d) a PEG lipid in an amount from about 0.5-1.8 mol % of the lipid component;
wherein the ionizable lipid is a compound of Formula (I)

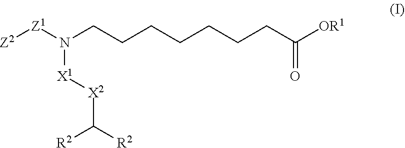

wherein
X¹ is C₆₋₇ alkylene;
X² is

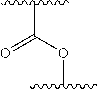 [SMILES: *OC(*)=O]

or absent, provided that if X² is

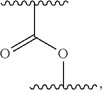 [SMILES: *OC(*)=O]

R² is not alkoxy;
Z¹ is C₂₋₃ alkylene;
Z² is selected from —OH, —NHC(═O)OCH₃, and —NHS(═O)₂CH₃;
R¹ is C₇₋₉ unbranched alkyl or C₇₋₁₁ unbranched alkynyl; and
each R² is independently C₈ alkyl or C₈ alkoxy;
   or a salt thereof.

In some embodiments, the LNP composition comprises:
a biologically active agent; and
a lipid component, wherein the lipid component comprises:
a) an ionizable lipid in an amount from about 25-50 mol % of the lipid component;
b) a neutral lipid in an amount from about 7-25 mol % of the lipid component;
c) a helper lipid in an amount from about 39-65 mol % of the lipid component; and
d) a PEG lipid in an amount from about 0.8-1.8 mol % of the lipid component;
wherein the ionizable lipid is a compound of Formula (I)

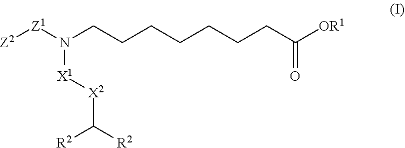

wherein
X¹ is C₆₋₇ alkylene;
X² is

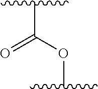 [SMILES: *OC(*)=O]

or absent, provided that if X² is

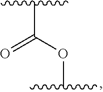 [SMILES: *OC(*)=O]

R² is not alkoxy;
Z¹ is C₂₋₃ alkylene;
Z² is selected from —OH, —NHC(═O)OCH₃, and —NHS(═O)₂CH₃;
R¹ is C₇₋₉ unbranched alkyl; and
each R² is independently C₈ alkyl or C₈ alkoxy;
   or a salt thereof.

In certain embodiments, the amount of the ionizable lipid is from about 30-45 mol % of the lipid component, the amount of the neutral lipid is from about 10-15 mol % of the lipid component, the amount of the helper lipid is from about 39-59 mol % of the lipid component, and the amount of the PEG lipid is from about 1-1.5 mol % of the lipid component.

In some embodiments, the amount of the ionizable lipid is about 30 mol % of the lipid component, the amount of the neutral lipid is about 10 mol % of the lipid component, the amount of the helper lipid is about 59 mol % of the lipid component, and the amount of the PEG lipid is about 1-1.5 mol % of the lipid component.

In some embodiments, the amount of the ionizable lipid is about 40 mol % of the lipid component, the amount of the neutral lipid is about 15 mol % of the lipid component, the amount of the helper lipid is about 43.5 mol % of the lipid component, and the amount of the PEG lipid is about 1.5 mol % of the lipid component.

In some embodiments, the amount of the ionizable lipid is about 50 mol % of the lipid component, the amount of the neutral lipid is about 10 mol % of the lipid component, the amount of the helper lipid is about 39 mol % of the lipid component, and the amount of the PEG lipid is about 1 mol % of the lipid component.

In certain embodiments, the ionizable lipid is

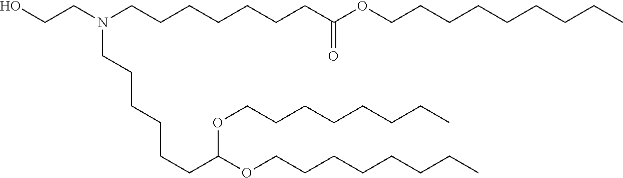 [SMILES: CCCCCCCCCOC(=O)CCCCCCCN(CCO)CCCCCCC(OCCCCCCCC)OCCCCCCCC]

or a salt thereof, the neutral lipid is DSPC; the helper lipid is cholesterol; and the PEG lipid is 1,2-dimyristoyl-rac-glycero-3-methoxypolyethylene glycol-2000.

In certain embodiments, the LNPs have a Z-average diameter of less than about 145 nm, for example, less than about 100 nm, less than about 95 nm, or less than about 90 nm. In certain embodiments, the LNPs have a number-average diameter of greater than about 45 nm, for example, greater than about 50 nm.

In certain embodiments, the LNPs have a polydispersity index of about 0.005 to about 0.75, for example about 0.005 to about 0.1.

In some embodiments, the N/P ratio of the LNP composition is from about 5 to about 7, preferably, about 6.

In certain embodiments, the disclosure relates to any LNP composition described herein wherein the nucleic acid component is an RNA component. In some embodiments, the RNA component comprises an mRNA. In preferred embodiments, the disclosure relates to any LNP composition described herein, wherein the RNA component comprises an RNA-guided DNA-binding agent, for example a Cas nuclease mRNA, such as a Class 2 Cas nuclease mRNA, or a Cas9 nuclease mRNA.

In certain embodiments, the disclosure relates to any LNP composition described herein, wherein the mRNA is a modified mRNA. In some embodiments, the disclosure relates to any LNP composition described herein, wherein the RNA component comprises a gRNA nucleic acid. In certain embodiments, the disclosure relates to any LNP composition described herein, wherein the gRNA nucleic acid is a gRNA.

In certain preferred embodiments, the disclosure relates to an LNP composition described herein, wherein the RNA component comprises a Class 2 Cas nuclease mRNA and a gRNA. In certain embodiments, the disclosure relates to any LNP composition described herein, wherein the gRNA nucleic acid is or encodes a dual-guide RNA (dgRNA). In certain embodiments, the disclosure relates to any LNP composition described herein, wherein the gRNA nucleic acid is or encodes a single-guide RNA (sgRNA).

In certain embodiments, the disclosure relates to an LNP composition described herein, comprising a guide RNA nucleic acid and a Class 2 Cas nuclease mRNA, where the ratio of the mRNA to the guide RNA nucleic acid is from about 2:1 to 1:4 by weight, preferably about 1:1 by weight.

In certain embodiments, the disclosure relates to any LNP composition described herein, wherein the gRNA is a modified gRNA, for example the modified gRNA comprises a modification at one or more of the first five nucleotides at a 5′ end, or the modified gRNA comprises a modification at one or more of the last five nucleotides at a 3′ end, or both.

In certain embodiments, the disclosure relates to a method of delivering a biologically active agent to a cell, comprising contacting a cell with an LNP composition described herein.

In certain embodiments, the disclosure relates to a method of cleaving DNA, comprising contacting a cell with an LNP composition described herein. In certain embodiments, the cleaving step comprises introducing a single stranded DNA nick. In other embodiments, the cleaving step comprises introducing a double-stranded DNA break. In certain embodiments, the LNP composition comprises a Class 2 Cas mRNA and a gRNA nucleic acid. In certain embodiments, the methods further comprise introducing at least one template nucleic acid into the cell.

In certain embodiments, the disclosure relates to any method of gene editing described herein, comprising administering the LNP composition to an animal, for example a human. In certain embodiments, the method comprises administering the LNP composition to a cell, such as a eukaryotic cell, and in particular a human cell. In some embodiments, the cell is a type of cell useful in a therapy, for example, adoptive cell therapy (ACT). Examples of ACT include autologous and allogeneic cell therapies. In some embodiments, the cell is a stem cell, such as a hematopoietic stem cell, an induced pluripotent stem cell, or another multipotent or pluripotent cell. In some embodiments, the cell is a stem cell, for example, a mesenchymal stem cell that can develop into a bone, cartilage, muscle, or fat cell. In some embodiments, the stem cells comprise ocular stem cells. In certain embodiments, the cell is selected from mesenchymal stem cells, hematopoietic stem cells (HSCs), mononuclear cells, endothelial progenitor cells (EPCs), neural stem cells (NSCs), limbal stem cells (LSCs), tissue-specific primary cells or cells derived therefrom (TSCs), induced pluripotent stem cells (iPSCs), ocular stem cells, pluripotent stem cells (PSCs), embryonic stem cells (ESCs), and cells for organ or tissue transplantations.

In certain embodiments, the cell is a liver cell. In other embodiments, the cell is an immune cell, for example, a leukocyte or a lymphocyte, preferably a lymphocyte, even more preferably, a T cell, a B cell, or an NK cell, most preferably an activated T cell or a non-activated T cell.

In certain embodiments, the disclosure relates to any method of gene editing described herein, comprising administering the mRNA formulated in a first LNP composition and a second LNP composition comprising one or more of an mRNA, a gRNA, and a gRNA nucleic acid. In some embodiments, the first and second LNP compositions are administered simultaneously. In other embodiments, the first and second LNP compositions are administered sequentially. In certain embodiments, the mRNA and the gRNA nucleic acid are formulated in a single LNP composition. In some embodiments, the first LNP composition comprises a first gRNA and the second LNP composition comprises a second gRNA, wherein the first and second gRNAs comprise different guide sequences that are complementary to different targets.

In certain embodiments, the disclosure relates to any method of gene editing described herein, wherein the cell is contacted with the LNP composition in vitro. In certain embodiments, the disclosure relates to any method of gene editing described herein, wherein the cell is contacted with the LNP composition ex vivo. In certain embodiments, the disclosure relates to any method of gene editing described herein, comprising contacting a tissue of an animal with the LNP.

In certain embodiments, the disclosure relates to any method of gene editing described herein, wherein the gene editing results in a gene knockout.

In some embodiments, the disclosure relates to any method of gene editing described herein, wherein the gene editing results in a gene correction.

In certain embodiments, the disclosure relates to any method of gene editing described herein, wherein the gene editing results in an insertion. In some embodiments, the insertion is a gene insertion.

Provided herein are methods for genetically engineering T cells in vitro that overcome the hurdles of prior processes. In some embodiments, naïve T cells are contacted in vitro with at least one lipid composition and genetically modified. In some embodiments, non-activated T cells are contacted in vitro with two or more lipid compositions and genetically modified. In some embodiments, activated T cells are contacted in vitro with two or more lipid compositions and genetically modified. In some embodiments, T cells are modified in a pre-activation step, comprising contacting the (non-activated) T cell with one or more lipid compositions, followed by activating the T cell, followed by further modifications to the T cell in a post-activation step, comprising contacting the activated T cell with one or more lipid compositions. In some embodiments, the non-activated T cell is contacted with one, two, or three lipid compositions. In some embodiments, the activated T cell is contacted with one to twelve lipid compositions. In some embodiments, the activated T cell is contacted with one to eight lipid compositions, optionally one to four lipid compositions. In some embodiments, the activated T cell is contacted with one to six lipid compositions. In some embodiments, the T cell is contacted with two lipid compositions. In some embodiments, the T cell is contacted with three lipid compositions. In some embodiments, the T cell is contacted with four lipid compositions. In some embodiments, the T cell is contacted with five lipid compositions. In some embodiments, the T cell is contacted with six lipid compositions. In some embodiments, the T cell is contacted with seven lipid compositions. In some embodiments, the T cell is contacted with eight lipid compositions. In some embodiments, the T cell is contacted with nine lipid compositions. In some embodiments, the T cell is contacted with ten lipid compositions. In some embodiments, the T cell is contacted with eleven lipid compositions. In some embodiments, the T cell is contacted with twelve lipid compositions. Such exemplary sequential administration (optionally with further sequential or simultaneous administration in the pre-activation step and post-activation step) of lipid compositions takes advantage of the activation status of the T cell and provides for unique advantages and healthier cells post-editing. In some embodiments, the genetically engineered T cells have the advantageous properties of high editing efficiency at each target site, increased post-editing survival rate, low toxicity despite the multiplicity of transfections, low translocations (e.g., no measurable target-target translocations), increased production of cytokines (e.g., IL-2, IFNγ, TNFα), continued proliferation with repeat stimulation (e.g., with repeat antigen stimulation), increased expansion, and/or expression of memory cell phenotype markers, including for example, early stem cells.

### BRIEF DESCRIPTION OF DRAWINGS

FIG. 1A is a graph showing the percentage of CD3− cells after delivering Cas9 mRNA and sgRNA to activated CD3+ T cells by using LNP compositions with Compound 3 having various ratios of lipid components.

FIG. 1B is a graph showing the percentage of CD3− cells after delivering Cas9 mRNA and sgRNA to non-activated CD3+ T cells by using LNP compositions with Compound 3 having various ratios of lipid components.

FIG. 2A is a graph showing the percentage of CD3− cells after delivering Cas9 mRNA and sgRNA to activated CD3+ T cells by using LNP compositions with Compound 3 having various ratios of lipid components.

FIG. 2B is a graph showing the percentage of CD3− cells after delivering Cas9 mRNA and sgRNA to non-activated CD3+ T cells by using LNP compositions with Compound 3 having various ratios of lipid components.

FIG. 3A is a graph showing the percentage of CD3− cells after delivering Cas9 mRNA and sgRNA to activated CD3+ T cells by using LNP compositions with Compound 1, Compound 3, and Compound 4, having a nominal mol % ratio of lipid components: 30% ionizable lipid, 10% DSPC, 59% cholesterol, and 1.5% PEG-2k-DMG, and comparative LNP compositions with Compound 1, Compound 3, and Compound 4, having a nominal mol % ratio of lipid components: 50% ionizable lipid, 10% DSPC, 38.5% cholesterol, and 1.5% PEG-2k-DMG.

FIG. 3B is a graph showing the percentage of CD3− cells after delivering Cas9 mRNA and sgRNA to non-activated CD3+ T cells by using LNP compositions with Compound 1, Compound 3, and Compound 4, having a nominal mol % ratio of lipid components: 30% ionizable lipid, 10% DSPC, 59% cholesterol, and 1.5% PEG-2k-DMG, and comparative LNP compositions with Compound 1, Compound 3, and Compound 4, having a nominal mol % ratio of lipid components: 50% ionizable lipid, 10% DSPC, 38.5% cholesterol, and 1.5% PEG-2k-DMG.

FIG. 4 is a graph showing the effect of various LNP composition concentrations on percent editing after delivering Cas9 mRNA and AAVS1-targeting sgRNA to NK cells by using LNP compositions having a nominal mol % ratio of lipid components: 30% ionizable lipid (Compound 3), 10% DSPC, 59% cholesterol, and 1.5% PEG-2k-DMG; 50% ionizable lipid (Compound 3), 10% DSPC, 38.5% cholesterol, and 1.5% PEG-2k-DMG; and 50% ionizable lipid (Compound 8), 10% DSPC, 38.5% cholesterol, and 1.5% PEG-2k-DMG.

FIG. 5A is a graph showing the effect of various LNP composition concentrations on percent editing after delivering Cas9 mRNA and AAVS1-targeting sgRNA to monocytes by using LNP compositions having a nominal mol % ratio of lipid components: 30% ionizable lipid (Compound 3), 10% DSPC, 59% cholesterol, and 1.5% PEG-2k-DMG; 50% ionizable lipid (Compound 3), 10% DSPC, 38.5% cholesterol, and 1.5% PEG-2k-DMG; and 50% ionizable lipid (Compound 8), 10% DSPC, 38.5% cholesterol, and 1.5% PEG-2k-DMG.

FIG. 5B is a graph showing the effect of various LNP composition concentrations on percent editing after delivering Cas9 mRNA and AAVS1-targeting sgRNA to macrophages by using LNP compositions having a nominal mol % ratio of lipid components: 30% ionizable lipid (Compound 3), 10% DSPC, 59% cholesterol, and 1.5% PEG-2k-DMG; 50% ionizable lipid (Compound 3), 10% DSPC, 38.5% cholesterol, and 1.5% PEG-2k-DMG; and 50% ionizable lipid (Compound 8), 10% DSPC, 38.5% cholesterol, and 1.5% PEG-2k-DMG.

FIG. 6 is a graph showing the effect of various LNP composition concentrations on percent editing after delivering Cas9 mRNA and AAVS1-targeting sgRNA to B cells by using LNP compositions having a nominal mol % ratio of lipid components: 30% ionizable lipid (Compound 3), 10% DSPC, 59% cholesterol, and 1.5% PEG-2k-DMG; 50% ionizable lipid (Compound 3), 10% DSPC, 38.5% cholesterol, and 1.5% PEG-2k-DMG; and 50% ionizable lipid (Compound 8), 10% DSPC, 38.5% cholesterol, and 1.5% PEG-2k-DMG.

### DETAILED DESCRIPTION

The present disclosure provides lipid compositions useful for delivering biologically active agents, including nucleic acids, such as CRISPR/Cas component RNAs (mRNA and/or gRNA) (the “cargo”), to a cell, and methods for preparing and using such compositions. Such lipid compositions include an ionizable lipid, a neutral lipid, a PEG lipid, and a helper lipid. In some embodiments, the ionizable lipid is a compound of Formula (I) or (II), as defined herein. In certain embodiments, the lipid compositions may comprise a biologically active agent, e.g. an RNA component. In certain embodiments, the RNA component includes an mRNA. In some embodiments the mRNA is an mRNA encoding a Class 2 Cas nuclease. In certain embodiments, the RNA component includes a gRNA and optionally an mRNA encoding a Class 2 Cas nuclease. In some embodiments, the lipid compositions are lipid nanoparticle (LNP) compositions. “Lipid nanoparticle” or “LNP” refers to, without limiting the meaning, a particle that comprises a plurality of (i.e., more than one) lipid components physically associated with each other by intermolecular forces.

Methods of gene editing and methods of making engineered cells using these lipid compositions are also provided. In some embodiments, LNP compositions, may be used to deliver a biologically active agent to a cell, a tissue, or an animal. In some embodiments, the cell is a eukaryotic cell, and in particular a human cell. In some embodiments, the cell is a liver cell. In some embodiments, the cell is a type of cell useful in a therapy, for example, adoptive cell therapy (ACT), such as autologous and allogeneic cell therapies. In some embodiments, the cell is a stem cell, such as a hematopoietic stem cell, an induced pluripotent stem cell, or another multipotent or pluripotent cell. In some embodiments, the cell is a stem cell, for example, a mesenchymal stem cell that can develop into a bone, cartilage, muscle, or fat cell. In some embodiments, the stem cells comprise ocular stem cells. In certain embodiments, the cell is selected from mesenchymal stem cells, hematopoietic stem cells (HSCs), mononuclear cells, endothelial progenitor cells (EPCs), neural stem cells (NSCs), limbal stem cells (LSCs), tissue-specific primary cells or cells derived therefrom (TSCs), induced pluripotent stem cells (iPSCs), ocular stem cells, pluripotent stem cells (PSCs), embryonic stem cells (ESCs), and cells for organ or tissue transplantations.

In some embodiments, the cell is an immune cell, such as a leukocyte or a lymphocyte. In preferred embodiments, the immune cell is a lymphocyte. In certain embodiments, the lymphocyte is a T cell, a B cell, or an NK cell. In preferred embodiments, the lymphocyte is a T cell. In certain embodiments, the lymphocyte is an activated T cell. In certain embodiments, the lymphocyte is a non-activated T cell.

In some embodiments, the LNP compositions and methods provided herein result in an editing efficiency of greater than about 80%, greater than about 90%, or greater than about 95%. In some embodiments, the LNP compositions and methods result in an editing efficiency of about 80-95%, about 90-95%, about 80-99%, about 90-99%, or about 95-99%.

Ionizable Lipids

The disclosure provides ionizable lipids that can be used in LNP compositions.

In some embodiments, the ionizable lipid is a compound of Formula (I)

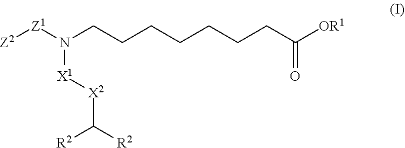

wherein
X¹ is C₆₋₇ alkylene;
X² is

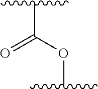 [SMILES: *OC(*)=O]

or absent, provided that if X² is

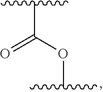 [SMILES: *OC(*)=O]

R² is not alkoxy;
Z¹ is C₂₋₃ alkylene;
Z² is selected from —OH, —NHC(═O)OCH₃, and —NHS(═O)₂CH₃;
R¹ is C₇₋₉ unbranched alkyl or C₇₋₁₁ unbranched alkynyl; and
each R² is independently C₈ alkyl or C₈ alkoxy;
   or a salt thereof.

In some embodiments, the ionizable lipid is a compound having a structure of Formula I

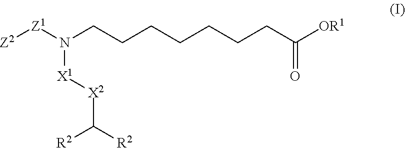

wherein
X¹ is C₆₋₇ alkylene;
X² is

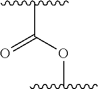 [SMILES: *OC(*)=O]

or absent, provided that if X² is

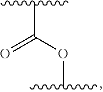 [SMILES: *OC(*)=O]

R² is not alkoxy;
Z¹ is C₂₋₃ alkylene;
Z² is selected from —OH, —NHC(═O)OCH₃, and —NHS(═O)₂CH₃;
R¹ is C₇₋₉ unbranched alkyl; and
each R² is independently C₈ alkyl or C₈ alkoxy;
or a salt thereof

In some embodiments, the ionizable lipid is a compound of Formula (II)

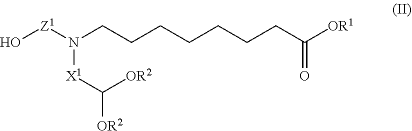

wherein
X¹ is C₆₋₇ alkylene;
Z₁ is C₂₋₃ alkylene;
R¹ is C₇₋₉ unbranched alkyl; and
each R² is C₈ alkyl;
or a salt thereof.

In certain embodiments, X¹ is C₆ alkylene. In other embodiments, X¹ is C₇ alkylene.

In certain embodiments, Z¹ is a direct bond and R⁵ and R⁶ are each C₈ alkoxy. In other embodiments, Z¹ is C₃ alkylene and R⁵ and R⁶ are each C₆ alkyl.

In certain embodiments, X² is

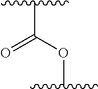 [SMILES: *OC(*)=O]

and R² is not alkoxy. In other embodiments, X² is absent.

In certain embodiments, Z¹ is C₂ alkylene; In other embodiments, Z¹ is C₃ alkylene.

In certain embodiments, Z² is —OH. In other embodiments, Z² is —NHC(═O)OCH₃. In other embodiments, Z² is —NHS(═O)₂CH₃.

In certain embodiments, R¹ is C₇ unbranched alkylene. In other embodiments, R¹ is C₈ branched or unbranched alkylene. In other embodiments, R¹ is C₉ branched or unbranched alkylene.

In certain embodiments, the ionizable lipid is a salt.

Representative compounds of Formula (I) include:

| Compound | | |
|---|---|---|
| Number | Compound | |
| 1 | 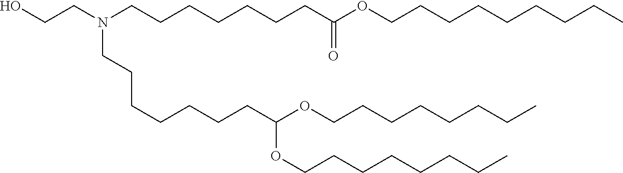 [SMILES: CCCCCCCCCOC(=O)CCCCCCCN(CCO)CCCCCCCC(OCCCCCCCC)OCCCCCCCC] | |
| 2 | 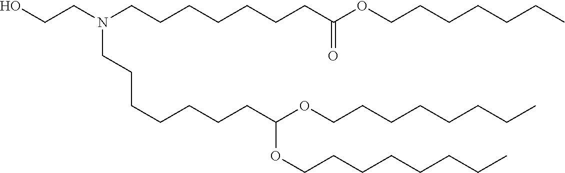 [SMILES: CCCCCCCCOC(CCCCCCCN(CCO)CCCCCCCC(=O)OCCCCCCC)OCCCCCCCC] | |
| 3 | 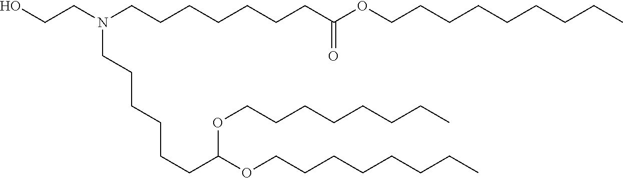 [SMILES: CCCCCCCCCOC(=O)CCCCCCCN(CCO)CCCCCCC(OCCCCCCCC)OCCCCCCCC] | |
| 4 | 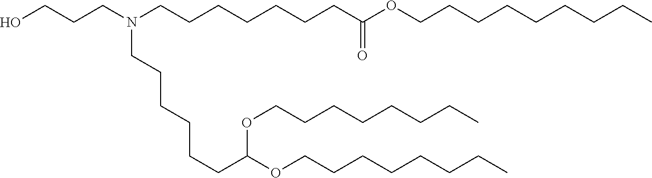 [SMILES: CCCCCCCCCOC(=O)CCCCCCCN(CCCO)CCCCCCC(OCCCCCCCC)OCCCCCCCC] | |
| 5 | 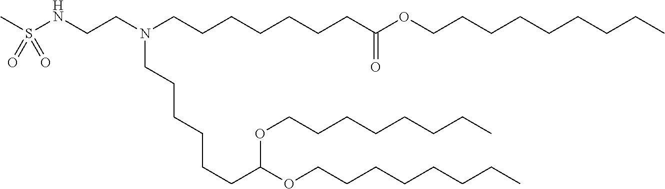 [SMILES: CCCCCCCCCOC(=O)CCCCCCCN(CCCCCCC(OCCCCCCCC)OCCCCCCCC)CCNS(C)(=O)=O] | |
| 6 | 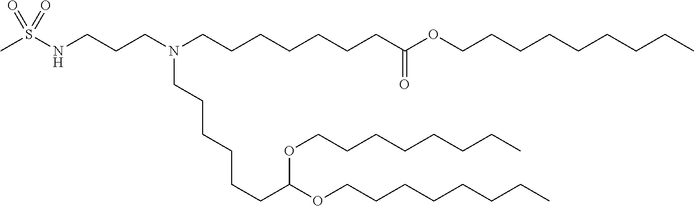 [SMILES: CCCCCCCCCOC(=O)CCCCCCCN(CCCCCCC(OCCCCCCCC)OCCCCCCCC)CCCNS(C)(=O)=O] | |
| 7 | | 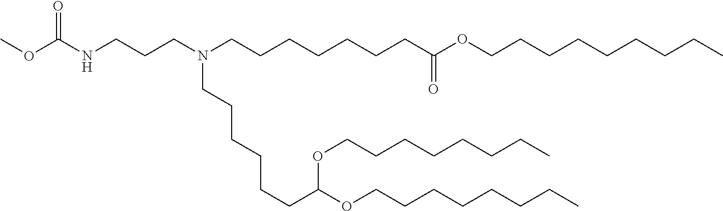 [SMILES: CCCCCCCCCOC(=O)CCCCCCCN(CCCCCCC(OCCCCCCCC)OCCCCCCCC)CCCNC(=O)OC] |

or a salt thereof, such as a pharmaceutically acceptable salt thereof. The compounds may be synthesized according to the methods set forth in WO2020/072605 (e.g., pp 69-101) and Mol. Ther. 2018, 26(6), 1509-1519 (“Sabnis”), each of which is incorporated by reference in its entirety.

The compounds of Formula (I) or (II) of the present disclosure may form salts depending upon the pH of the medium they are in. For example, in a slightly acidic medium, the compounds of Formula (I) or (II) may be protonated and thus bear a positive charge. Conversely, in a slightly basic medium, such as, for example, blood where pH is approximately 7.35, the compounds of Formula (I) or (II) may not be protonated and thus bear no charge. In some embodiments, the compounds of Formula (I) or (II) of the present disclosure may be predominantly protonated at a pH of at least about 9. In some embodiments, the compounds of Formula (I) or (II) of the present disclosure may be predominantly protonated at a pH of at least about 10.

The pH at which a compound of Formula (I) or (II) is predominantly protonated is related to its intrinsic pKa. In some embodiments, a salt of a compound of Formula (I) or (II) of the present disclosure has a pKa in the range of from about 5.1 to about 8.0, even more preferably from about 5.5 to about 7.6. In some embodiments, a salt of a compound of Formula (I) or (II) of the present disclosure has a pKa in the range of from about 5.7 to about 8, from about 5.7 to about 7.6, from about 6 to about 8, from about 6 to about 7.5, from about 6 to about 7, from about 6 to about 6.5, or from about 6 to about 6.3. In some embodiments, a salt of a compound of Formula (I) or (II) of the present disclosure has a pKa of about 6.0, about 6.1, about 6.1, about 6.2, about 6.3, about 6.4, about 6.6, or about 6.6. Alternatively, a salt of a compound of Formula (I) or (II) of the present disclosure has a pKa in the range of from about 6 to about 8. The pKa of a salt of a compound of Formula (I) or (II) can be an important consideration in formulating LNPs, as it has been found that LNPs formulated with certain lipids having a pKa ranging from about 5.5 to about 7.0 are effective for delivery of cargo in vivo, e.g. to the liver. Further, it has been found that LNPs formulated with certain lipids having a pKa ranging from about 5.3 to about 6.4 are effective for delivery in vivo, e.g. to tumors. See, e.g., WO 2014/136086. In some embodiments, the ionizable lipids are positively charged at an acidic pH but neutral in the blood.

Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine di stearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In certain embodiments, the neutral phospholipid is selected from di stearoylphosphatidylcholine (DSPC) and dimyristoyl phosphatidyl ethanolamine (DMPE), preferably di stearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In certain embodiments, the helper lipid may be cholesterol or a derivative thereof, such as cholesterol hemisuccinate.

In some embodiments, the LNP compositions include polymeric lipids, such as PEG lipids which can affect the length of time the nanoparticles can exist in vivo or ex vivo (e.g., in the blood or medium). PEG lipids may assist in the formulation process by, for example, reducing particle aggregation and controlling particle size. PEG lipids used herein may modulate pharmacokinetic properties of the LNPs. Typically, the PEG lipid comprises a lipid moiety and a polymer moiety based on PEG (sometimes referred to as poly(ethylene oxide)) (a PEG moiety). PEG lipids suitable for use in a lipid composition with a compound of Formula (I) or (II) of the present disclosure and information about the biochemistry of such lipids can be found in Romberg et al., Pharmaceutical Research 25(1), 2008, pp. 55-71 and Hoekstra et al., Biochimica et Biophysica Acta 1660 (2004) 41-52. Additional suitable PEG lipids are disclosed, e.g., in WO 2015/095340 (p. 31, line 14 top. 37, line 6), WO 2006/007712, and WO 2011/076807 (“stealth lipids”), each of which is incorporated by reference in its entirety.

In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetric.

Unless otherwise indicated, the term “PEG” as used herein means any polyethylene glycol or other polyalkylene ether polymer, such as an optionally substituted linear or branched polymer of ethylene glycol or ethylene oxide. In certain embodiments, the PEG moiety is unsubstituted. Alternatively, the PEG moiety may be substituted, e.g., by one or more alkyl, alkoxy, acyl, hydroxy, or aryl groups. For example, the PEG moiety may comprise a PEG copolymer such as PEG-polyurethane or PEG-polypropylene (see, e.g., J. Milton Harris, Poly(ethylene glycol) chemistry: biotechnical and biomedical applications (1992)); alternatively, the PEG moiety may be a PEG homopolymer. In certain embodiments, the PEG moiety has a molecular weight of from about 130 to about 50,000, such as from about 150 to about 30,000, or even from about 150 to about 20,000. Similarly, the PEG moiety may have a molecular weight of from about 150 to about 15,000, from about 150 to about 10,000, from about 150 to about 6,000, or even from about 150 to about 5,000. In certain preferred embodiments, the PEG moiety has a molecular weight of from about 150 to about 4,000, from about 150 to about 3,000, from about 300 to about 3,000, from about 1,000 to about 3,000, or from about 1,500 to about 2,500.

In certain preferred embodiments, the PEG moiety is a “PEG-2K,” also termed “PEG 2000,” which has an average molecular weight of about 2,000 daltons. PEG-2K is represented herein by the following formula (III),

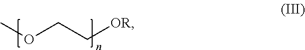

wherein n is about 45, meaning that the number averaged degree of polymerization comprises about 45 subunits. However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl, such as methyl.

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-di stearoylglycamide, PEG-cholesterol (1-[8′-(Cholest-5-en-3 [beta]-oxy)carboxamido-3′,6′-dioxaoctanyl] carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMPE), or 1,2-dimyristoyl-rac-glycero-3-methoxypolyethylene glycol-2000 (PEG2k-DMG), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In certain such embodiments, the PEG lipid may be PEG2k-DMG. In some embodiments, the PEG lipid may be PEG2k-DSG. In other embodiments, the PEG lipid may be PEG2k-DSPE. In some embodiments, the PEG lipid may be PEG2k-DMA. In yet other embodiments, the PEG lipid may be PEG2k-C-DMA. In certain embodiments, the PEG lipid may be compound S027, disclosed in WO2016/010840 (paragraphs to [00244]). In some embodiments, the PEG lipid may be PEG2k-DSA. In other embodiments, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

In preferred embodiments, the PEG lipid includes a glycerol group. In preferred embodiments, the PEG lipid includes a dimyristoylglycerol (DMG) group. In preferred embodiments, the PEG lipid comprises PEG-2k. In preferred embodiments, the PEG lipid is a PEG-DMG. In preferred embodiments, the PEG lipid is a PEG-2k-DMG. In preferred embodiments, the PEG lipid is 1,2-dimyristoyl-rac-glycero-3-methoxypolyethylene glycol-2000. In preferred embodiments, the PEG-2k-DMG is 1,2-dimyristoyl-rac-glycero-3-methoxypolyethylene glycol-2000.

### Lipid Compositions

Described herein are lipid compositions comprising at least one compound of Formula (I) or (II), or a salt thereof (e.g., a pharmaceutically acceptable salt thereof), at least one helper lipid, at least one neutral lipid, and at least one polymeric lipid. In some embodiments, the lipid composition comprises at least one compound of Formula (I) or (II), or a salt thereof, at least one neutral lipid, at least one helper lipid, and at least one PEG lipid. In some embodiments, the neutral lipid is DSPC or DPME. In some embodiments, the helper lipid is cholesterol, 5-heptadecylresorcinol, or cholesterol hemi succinate.

In preferred embodiments, the ionizable lipid is

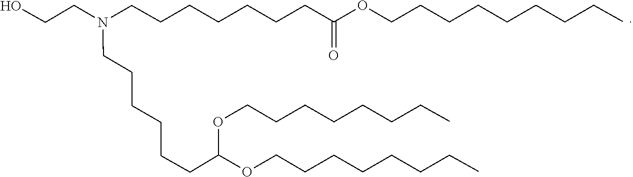 [SMILES: CCCCCCCCCOC(=O)CCCCCCCN(CCO)CCCCCCC(OCCCCCCCC)OCCCCCCCC]

In preferred embodiments, the neutral lipid is DSPC. In preferred embodiments, the helper lipid is cholesterol. In preferred embodiments, the PEG lipid is 1,2-dimyristoyl-rac-glycero-3-methoxypolyethylene glycol-2000. In particularly preferred embodiments, the ionizable lipid is

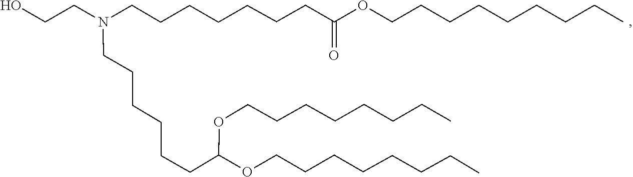 [SMILES: CCCCCCCCCOC(=O)CCCCCCCN(CCO)CCCCCCC(OCCCCCCCC)OCCCCCCCC]

the neutral lipid is DSPC, the helper lipid is cholesterol, and the PEG lipid is 1,2-dimyristoyl-rac-glycero-3-methoxypolyethylene glycol-2000.

In some embodiments, the lipid composition further comprises one or more additional lipid components.

In some embodiments, the lipid composition is in the form of a liposome. In preferred embodiments, the lipid composition is in the form of a lipid nanoparticle (LNP). In certain embodiments the lipid composition is suitable for delivery in vivo. In certain embodiments the lipid composition is suitable for delivery to an organ, such as the liver. In certain embodiments the lipid composition is suitable for delivery to a tissue ex vivo. In certain embodiments the lipid composition is suitable for delivery to a cell in vitro.

Lipid compositions comprising lipids of Formula (I) or (II), or a pharmaceutically acceptable salt thereof, may be in various forms, including, but not limited to, particle forming delivery agents including microparticles, nanoparticles and transfection agents that are useful for delivering various molecules to cells. Specific compositions are effective at transfecting or delivering biologically active agents. Preferred biologically active agents are nucleic acids such as RNAs. In further embodiments, the biologically active agent is chosen from mRNA and gRNA. The gRNA may be a dgRNA or an sgRNA. In certain embodiments, the cargo includes an mRNA encoding an RNA-guided DNA-binding agent (e.g. a Cas nuclease, a Class 2 Cas nuclease, or Cas9), a gRNA or a nucleic acid encoding a gRNA, or a combination of mRNA and gRNA.

Exemplary compounds of Formula (I) or (II) for use in the above lipid compositions are given in WO2020/072605, which is incorporated by reference in its entirety. In certain embodiments, the compound of Formula (I) is Compound 1. In certain embodiments, the compound of Formula (I) is Compound 2. In certain embodiments, the compound of Formula (I) is Compound 3. In certain embodiments, the compound of Formula (I) is Compound 4. In certain embodiments, the compound of Formula (I) is Compound 5. In certain embodiments, the compound of Formula (I) is Compound 6. In certain embodiments, the compound of Formula (I) is Compound 7.

The compositions will generally, but not necessarily, include one or more pharmaceutically acceptable excipients. The term “excipient” includes any ingredient other than the compound(s) of the disclosure, the other lipid component(s) and the biologically active agent. An excipient may impart either a functional (e.g. drug release rate controlling) and/or a non-functional (e.g. processing aid or diluent) characteristic to the compositions. The choice of excipient will to a large extent depend on factors such as the particular mode of administration, the effect of the excipient on solubility and stability, and the nature of the dosage form.

Parenteral formulations are typically aqueous or oily solutions or suspensions. Where the formulation is aqueous, excipients such as sugars (including but not restricted to glucose, mannitol, sorbitol, etc.) salts, carbohydrates and buffering agents (preferably to a pH of from 3 to 9), but, for some applications, they may be more suitably formulated with a sterile non-aqueous solution or as a dried form to be used in conjunction with a suitable vehicle such as sterile, pyrogen-free water (WFI).

LNP Compositions

The lipid compositions may be provided as LNP compositions, and LNP compositions described herein may be provided as lipid compositions. Lipid nanoparticles may be, e.g., microspheres (including unilamellar and multilamellar vesicles, e.g. “liposomes”—lamellar phase lipid bilayers that, in some embodiments are substantially spherical, and, in more particular embodiments can comprise an aqueous core, e.g., comprising a substantial portion of RNA molecules), a dispersed phase in an emulsion, micelles or an internal phase in a suspension.

Described herein are LNP compositions comprising at least one compound of Formula (I) or (II), or a salt thereof (e.g., a pharmaceutically acceptable salt thereof), at least one helper lipid, at least one neutral lipid, and at least one polymeric lipid. In some embodiments, the LNP composition comprises at least one compound of Formula (I) or (II), or a pharmaceutically acceptable salt thereof, at least one neutral lipid, at least one helper lipid, and at least one PEG lipid. In some embodiments, the neutral lipid is DSPC or DPME. In some embodiments, the helper lipid is cholesterol, 5-heptadecylresorcinol, or cholesterol hemisuccinate.

In preferred embodiments, the ionizable lipid is

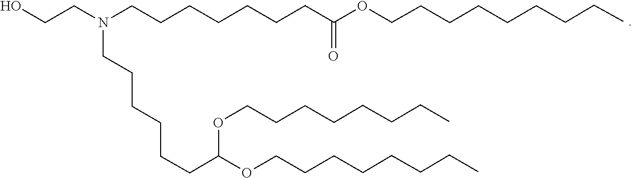 [SMILES: CCCCCCCCCOC(=O)CCCCCCCN(CCO)CCCCCCC(OCCCCCCCC)OCCCCCCCC]

In preferred embodiments, the neutral lipid is DSPC. In preferred embodiments, the helper lipid is cholesterol. In preferred embodiments, the PEG lipid is 1,2-dimyristoyl-rac-glycero-3-methoxypolyethylene glycol-2000. In particularly preferred embodiments, the ionizable lipid is

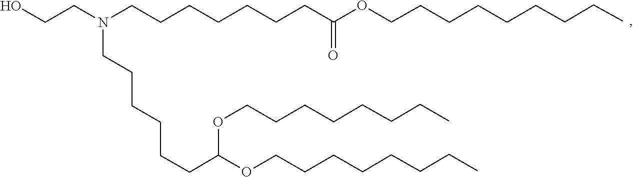 [SMILES: CCCCCCCCCOC(=O)CCCCCCCN(CCO)CCCCCCC(OCCCCCCCC)OCCCCCCCC]

the neutral lipid is DSPC, the helper lipid is cholesterol, and the PEG lipid is 1,2-dimyristoyl-rac-glycero-3-methoxypolyethylene glycol-2000.

Embodiments of the present disclosure provide lipid compositions described according to the respective molar ratios of the component lipids in the composition. All mol % numbers are given as a fraction of the lipid component of the lipid composition or, more specifically, the LNP compositions. In some embodiments, the lipid mol % of a lipid relative to the lipid component will be ±30%, ±25%, ±20%, ±15%, ±10%, ±5%, or ±2.5% of the specified, nominal, or actual mol % of the lipid. In some embodiments, the lipid mol % of a lipid relative to the lipid component will be ±4 mol %, ±3 mol %, ±2 mol %, ±1.5 mol %, ±1 mol %, ±0.5 mol %, ±0.25 mol %, or ±0.05 mol % of the specified, nominal, or actual mol % of the lipid component. In certain embodiments, the lipid mol % will vary by less than 15%, less than 10%, less than 5%, less than 1%, or less than 0.5% from the specified, nominal, or actual mol % of the lipid. In some embodiments, the mol % numbers are based on nominal concentration. As used herein, “nominal concentration” refers to concentration based on the input amounts of substances combined to form a resulting composition. For example, if 100 mg of solute is added to 1 L water, the nominal concentration is 100 mg/L. In some embodiments, the mol % numbers are based on actual concentration, e.g., concentration determined by an analytic method. In some embodiments, actual concentration of the lipids of the lipid component may be determined, for example, from chromatography, such as liquid chromatography, followed by a detection method, such as charged aerosol detection. In some embodiments, actual concentration of the lipids of the lipid component may be characterized by lipid analysis, AF4-MALS, NTA, and/or cryo-EM. All mol % numbers are given as a percentage of the lipids of the lipid component.

Embodiments of the present disclosure provide LNP compositions described according to the respective molar ratios of the lipids of the lipid component. In certain embodiments, the amount of the ionizable lipid is from about 25 mol % to about 50 mol %; the amount of the neutral lipid is from about 7 mol % to about 25 mol %; the amount of the helper lipid is from about 39 mol % to about 65 mol %; and the amount of the PEG lipid is from about 0.8 mol % to about 1.8 mol %. In certain embodiments, the amount of the ionizable lipid is from about 27-40 mol % of the lipid component; the amount of the neutral lipid is from about 10-20 mol % of the lipid component; the amount of the helper lipid is from about 50-60 mol % of the lipid component; and the amount of the PEG lipid is from about 0.9-1.6 mol % of the lipid component. In certain embodiments, the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component. In certain embodiments, the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component. In certain embodiments, the ionizable lipid is about 30 mol % of the lipid component; the amount of the neutral lipid is about 10 mol % of the lipid component; the amount of the helper lipid is about 59 mol % of the lipid component; and the amount of the PEG lipid is about 1-1.5 mol % of the lipid component. In certain embodiments, the amount of the ionizable lipid is about 40 mol % of the lipid component; the amount of the neutral lipid is about 15 mol % of the lipid component; the amount of the helper lipid is about 43.5 mol % of the lipid component; and the amount of the PEG lipid is about 1.5 mol % of the lipid component. In certain embodiments, the amount of the ionizable lipid is about 50 mol % of the lipid component; the amount of the neutral lipid is about 10 mol % of the lipid component; the amount of the helper lipid is about 39 mol % of the lipid component; and the amount of the PEG lipid is about 1 mol % of the lipid component.

In certain embodiments, the amount of the ionizable lipid is about 20-55 mol %, about 20-45 mol %, about 20-40 mol %, about 27-40 mol %, about 27-45 mol %, about 27-55 mol %, about 30-40 mol %, about 30-45 mol %, about 30-55 mol %, about 30 mol % about 40 mol %, or about 50 mol %. In additional embodiments, the amount of the ionizable lipid is about 20-55 mol %, about 20-50 mol %, about 20-45 mol %, about 20-43 mol %, about 20-40 mol %, about 20-38 mol %, about 20-35 mol %, about 20-33 mol %, about 20-30 mol %, about 25-55 mol %, about 25-50 mol %, about 25-45 mol %, about 25-43 mol %, about 25-40 mol %, about 25-38 mol %, about 25-35 mol %, about 25-33 mol %, about 25-30 mol %, about 27-55 mol %, about 27-50 mol %, about 27-45 mol %, about 27-43 mol %, about 27-40 mol %, about 27-38 mol %, about 27-35 mol %, about 27-33 mol %, about 27-30 mol %, about 30-55 mol %, about 30-50 mol %, about 30-45 mol %, about 30-43 mol %, about 30-40 mol %, about 30-38 mol %, about 30-35 mol %, about 30-33 mol %, about 32-55 mol %, about 32-50 mol %, about 32-45 mol %, about 32-43 mol %, about 32-40 mol %, about 32-38 mol %, about 32-35 mol %, about 35-55 mol %, about 35-50 mol %, about 35-45 mol %, about 35-43 mol %, about 35-40 mol %, about 35-38 mol %, about 37-55 mol %, about 37-50 mol %, about 37-45 mol %, about 37-43 mol %, about 37-40 mol %, about 40-55 mol %, about 40-50 mol %, about 40-45 mol %, about 40-43 mol %, about 43-55 mol %, about 43-50 mol %, about 43-45 mol %, about 45-55 mol %, about 45-50 mol %, or about 50-55 mol %. In some embodiments, the mol % of the ionizable lipid may be about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, about 46 mol %, about 47 mol %, about 48 mol %, about 49 mol %, or about 50 mol %. In some embodiments, the ionizable lipid mol % relative to the lipid component will be ±30%, ±25%, ±20%, ±15%, ±10%, ±5%, or ±2.5% of the specified, nominal, or actual mol %. In some embodiments, the ionizable lipid mol % relative to the lipid component will be ±4 mol %, ±3 mol %, ±2 mol %, ±1.5 mol %, ±1 mol %, ±0.5 mol %, or ±0.25 mol % of the specified, nominal, or actual mol %. In certain embodiments, LNP inter-lot variability of the ionizable lipid mol % will be less than 15%, less than 10% or less than 5%. In some embodiments, the mol % numbers are based on nominal concentration. In some embodiments, the mol % numbers are based on actual concentration.

In certain embodiments, the amount of the neutral lipid is about 7-25 mol %, about 10-25 mol %, about 10-20 mol %, about 15-20 mol %, about 8-15 mol %, about 10-15 mol %, about 10 mol %, or about 15 mol %. In additional embodiments, the amount of the neutral lipid may be about 5-30 mol %, about 5-28 mol %, about 5-25 mol %, about 5-23 mol %, about 5-20 mol %, about 5-18 mol %, about 5-23 mol %, about 5-20 mol %, about 5-18 mol %, about 5-15 mol %, about 5-13 mol %, about 5-10 mol %, about 10-30 mol %, about 10-28 mol %, about 10-25 mol %, about 10-23 mol %, about 10-20 mol %, about 10-18 mol %, about 10-23 mol %, about 10-20 mol %, about 10-18 mol %, about 10-15 mol %, about 10-13 mol %, about 12-30 mol %, about 12-28 mol %, about 12-25 mol %, about 12-23 mol %, about 12-20 mol %, about 12-18 mol %, about 12-23 mol %, about 12-20 mol %, about 12-18 mol %, about 12-15 mol %, about 15-30 mol %, about 15-28 mol %, about 15-25 mol %, about 15-23 mol %, about 15-20 mol %, about 15-18 mol %, about 15-23 mol %, about 15-20 mol %, about 15-18 mol %, about 17-30 mol %, about 17-28 mol %, about 17-25 mol %, about 17-23 mol %, about 17-20 mol %, about 17-18 mol %, about 17-23 mol %, about 17-20 mol %, about 20-30 mol %, about 20-28 mol %, about 20-25 mol %, about 20-23 mol %, about 22-30 mol %, about 22-28 mol %, about 22-25 mol %, about 22-23 mol %, about 22-20 mol %, or about 22-18 mol %. In some embodiments, the mol % of the neutral lipid may be about 5 mol %, about 6 mol %, about 7 mol %, about 8 mol %, or about 9 mol % about 10 mol %, about 11 mol %, about 12 mol %, about 13 mol %, about 14 mol %, about 15 mol %, about 16 mol %, about 17 mol %, about 18 mol %, about 19 mol %, or about 20 mol %. In some embodiments, the neutral lipid mol % relative to the lipid component will be ±30%, ±25%, ±20%, ±15%, ±10%, ±5%, or ±2.5% of the specified, nominal, or actual neutral lipid mol %. In some embodiments, the neutral lipid mol % relative to the lipid component will be ±4 mol %, ±3 mol %, ±2 mol %, ±1.5 mol %, ±1 mol %, ±0.5 mol %, or ±0.25 mol % of the specified, nominal, or actual mol %. In certain embodiments, LNP inter-lot variability will be less than 15%, less than 10% or less than 5%. In some embodiments, the mol % numbers are based on nominal concentration. In some embodiments, the mol % numbers are based on actual concentration.

In certain embodiments, the amount of the helper lipid is about 39-65 mol %, about 39-59 mol %, about 40-60 mol %, about 40-65 mol %, about 40-59 mol %, about 43-65 mol %, about 43-60 mol %, about 43-59 mol %, or about 50-65 mol %, about 50-59 mol %, about 59 mol %, or about 43.5 mol %. In additional embodiments, the amount of the helper lipid may be about 30-70 mol %, about 32-70 mol %, about 35-70 mol %, about 38-70 mol %, about 40-70 mol %, about 42-70 mol %, about 45-70 mol %, about 48-70 mol %, about 50-70 mol %, about 52-70 mol %, about 55-70 mol %, about 58-70 mol %, about 60-70 mol %, about 30-65 mol %, about 32-65 mol %, about 35-65 mol %, about 38-65 mol %, about 40-65 mol %, about 42-65 mol %, about 45-65 mol %, about 48-65 mol %, about 50-65 mol %, about 52-65 mol %, about 55-65 mol %, about 58-65 mol %, about 60-65 mol %, about 30-60 mol %, about 32-60 mol %, about 35-60 mol %, about 38-60 mol %, about 40-60 mol %, about 42-60 mol %, about 45-60 mol %, about 48-60 mol %, about 50-60 mol %, about 52-60 mol %, about 55-60 mol %, about 58-60 mol %, about 30-58 mol %, about 32-58 mol %, about 35-58 mol %, about 38-58 mol %, about 40-58 mol %, about 42-58 mol %, about 45-58 mol %, about 48-58 mol %, about 50-58 mol %, about 52-58 mol %, about 55-58 mol %, about 30-55 mol %, about 32-55 mol %, about 35-55 mol %, about 38-55 mol %, about 40-55 mol %, about 42-55 mol %, about 45-55 mol %, about 48-55 mol %, about 50-55 mol %, about 52-55 mol %, about 30-53 mol %, about 32-53 mol %, about 35-53 mol %, about 38-53 mol %, about 40-53 mol %, about 42-53 mol %, about 45-53 mol %, about 48-53 mol %, about 50-53 mol %, about 30-50 mol %, about 32-50 mol %, about 35-50 mol %, about 38-50 mol %, about 40-50 mol %, about 42-50 mol %, about 45-50 mol %, about 48-50 mol %, about 30-48 mol %, about 32-48 mol %, about 35-48 mol %, about 38-48 mol %, about 40-48 mol %, about 42-48 mol %, about 45-48 mol %, about 30-45 mol %, about 32-45 mol %, about 35-45 mol %, about 38-45 mol %, about 40-45 mol %, about 42-45 mol %, about 30-43 mol %, about 32-43 mol %, about 35-43 mol %, about 38-43 mol %, about 40-43 mol %, about 30-40 mol %, about 32-40 mol %, about 35-40 mol %, about 38-40 mol %, about 30-38 mol %, about 32-38 mol %, about 35-38 mol %, or about 30-35 mol %. It is to be understood that about 39 mol % helper lipid does not include 38.5% helper lipid. In certain embodiments, the amount of the helper lipid is adjusted based on the amounts of the ionizable lipid, the neutral lipid, and/or the PEG lipid to bring the LNP composition to about 100 mol %. In some embodiments, the helper lipid mol % relative to the lipid component will be ±30%, ±25%, ±20%, ±15%, ±10%, ±5%, or ±2.5% of the specified, nominal, or actual helper lipid mol %. In some embodiments, the helper lipid mol % relative to the lipid component will be ±4 mol %, ±3 mol %, ±2 mol %, ±1.5 mol %, ±1 mol %, ±0.5 mol %, or ±0.25 mol % of the specified, nominal, or actual mol %. In certain embodiments, LNP inter-lot variability will be less than 15%, less than 10% or less than 5%. In some embodiments, the mol % numbers are based on nominal concentration. In some embodiments, the mol % numbers are based on actual concentration.

In certain embodiments, the amount of the PEG lipid is about 0.8-1.8 mol %, about 0.8-1.6 mol %, about 0.8-1.5 mol %, 0.9-1.8 mol %, about 0.9-1.6 mol %, about 0.9-1.5 mol %, 1-1.8 mol %, about 1-1.6 mol %, about 1-1.5 mol %, about 1 mol %, or about 1.5 mol %. In additional embodiments, the amount of the PEG lipid may be about 0.5-2.5 mol %, about 0.7-2.5 mol %, about 0.8-2.5 mol %, about 0.9-2.5 mol %, about 1-2.5 mol %, about 1.1-2.5 mol %, about 1.2-2.5 mol %, about 1.3-2.5 mol %, about 1.4-2.5 mol %, about 1.5-2.5 mol %, about 1.6-2.5 mol %, about 1.7-2.5 mol %, about 1.8-2.5 mol %, about 1.9-2.5 mol %, about 2-2.5 mol %, about 2.2-2.5 mol %, about 0.5-2.2 mol %, about 0.7-2.2 mol %, about 0.8-2.2 mol %, about 0.9-2.2 mol %, about 1-2.2 mol %, about 1.1-2.2 mol %, about 1.2-2.2 mol %, about 1.3-2.2 mol %, about 1.4-2.2 mol %, about 1.5-2.2 mol %, about 1.6-2.2 mol %, about 1.7-2.2 mol %, about 1.8-2.2 mol %, about 1.9-2.2 mol %, about 2-2.2 mol %, about 0.5-2 mol %, about 0.7-2 mol %, about 0.8-2 mol %, about 0.9-2 mol %, about 1-2 mol %, about 1.1-2 mol %, about 1.2-2 mol %, about 1.3-2 mol %, about 1.4-2 mol %, about 1.5-2 mol %, about 1.6-2 mol %, about 1.7-2 mol %, about 1.8-2 mol %, about 1.9-2 mol %, about 0.5-1.9 mol %, about 0.7-1.9 mol %, about 0.8-1.9 mol %, about 0.9-1.9 mol %, about 1-1.9 mol %, about 1.1-1.9 mol %, about 1.2-1.9 mol %, about 1.3-1.9 mol %, about 1.4-1.9 mol %, about 1.5-1.9 mol %, about 1.6-1.9 mol %, about 1.7-1.9 mol %, about 1.8-1.9 mol %, about 0.5-1.8 mol %, about 0.7-1.8 mol %, about 0.8-1.8 mol %, about 0.9-1.8 mol %, about 1-1.8 mol %, about 1.1-1.8 mol %, about 1.2-1.8 mol %, about 1.3-1.8 mol %, about 1.4-1.8 mol %, about 1.5-1.8 mol %, about 1.6-1.8 mol %, about 1.7-1.8 mol %, about 0.5-1.7 mol %, about 0.7-1.7 mol %, about 0.8-1.7 mol %, about 0.9-1.7 mol %, about 1-1.7 mol %, about 1.1-1.7 mol %, about 1.2-1.7 mol %, about 1.3-1.7 mol %, about 1.4-1.7 mol %, about 1.5-1.7 mol %, about 1.6-1.7 mol %, about 0.5-1.6 mol %, about 0.7-1.6 mol %, about 0.8-1.6 mol %, about 0.9-1.6 mol %, about 1-1.6 mol %, about 1.1-1.6 mol %, about 1.2-1.6 mol %, about 1.3-1.6 mol %, about 1.4-1.6 mol %, about 1.5-1.6 mol %, about 0.5-1.5 mol %, about 0.7-1.5 mol %, about 0.8-1.5 mol %, about 0.9-1.5 mol %, about 1-1.5 mol %, about 1.1-1.5 mol %, about 1.2-1.5 mol %, about 1.3-1.5 mol %, about 1.4-1.5 mol %, about 0.5-1.4 mol %, about 0.7-1.4 mol %, about 0.8-1.4 mol %, about 0.9-1.4 mol %, about 1-1.4 mol %, about 1.1-1.4 mol %, about 1.2-1.4 mol %, about 1.3-1.4 mol %, about 0.5-1.3 mol %, about 0.7-1.3 mol %, about 0.8-1.3 mol %, about 0.9-1.3 mol %, about 1-1.3 mol %, about 1.1-1.3 mol %, about 1.2-1.3 mol %, about 0.5-1.2 mol %, about 0.7-1.2 mol %, about 0.8-1.2 mol %, about 0.9-1.2 mol %, about 1-1.2 mol %, about 1.1-1.2 mol %, about 0.5-1.1 mol %, about 0.7-1.1 mol %, about 0.8-1.1 mol %, about 0.9-1.1 mol %, about 1-1.1 mol %, about 0.5-1 mol %, about 0.7-1 mol %, about 0.8-1 mol %, about 0.9-1 mol %, about 0.5-0.9 mol %, about 0.7-0.9 mol %, about 0.8-0.9 mol %, about 0.5-0.8 mol %, about 0.7-0.8 mol %, or about 0.5-0.7 mol %. In some embodiments, the mol % of the PEG lipid may be about 0.7 mol %, about 0.8 mol %, about 0.9 mol %, about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, or about 2.5 mol %. In some embodiments, the PEG lipid mol % relative to the lipid component will be ±30%, ±25%, ±20%, ±15%, ±10%, ±5%, or ±2.5% of the specified, nominal, or actual PEG lipid mol %. In some embodiments, the PEG lipid mol % relative to the lipid component will be ±4 mol %, ±3 mol %, ±2 mol %, ±1.5 mol %, ±1 mol %, ±0.5 mol %, or ±0.25 mol % of the specified, nominal, or actual mol %. In certain embodiments, LNP inter-lot variability will be less than 15%, less than 10% or less than 5%. In some embodiments, the mol % numbers are based on nominal concentration. In some embodiments, the mol % numbers are based on actual concentration.

In certain embodiments, the lipid compositions, such as LNP compositions, comprise a lipid component and a nucleic acid component (also referred to as an aqueous component), e.g. an RNA component and the molar ratio of compound of Formula (I) or (II) to nucleic acid can be measured. Embodiments of the present disclosure also provide lipid compositions having a defined molar ratio between the positively charged amine groups of pharmaceutically acceptable salts of the compounds of Formula (I) or (II) (N) and the negatively charged phosphate groups (P) of the nucleic acid to be encapsulated. This may be mathematically represented by the equation N/P. In some embodiments, a lipid composition, such as an LNP composition, may comprise a lipid component that comprises a compound of Formula (I) or (II) or a pharmaceutically acceptable salt thereof; and a nucleic acid component, wherein the N/P ratio is about 3 to 10. In some embodiments, an LNP composition may comprise a lipid component that comprises a compound of Formula (I) or (II) or a pharmaceutically acceptable salt thereof; and an RNA component, wherein the N/P ratio is about 3 to 10. For example, the N/P ratio may be about 4-7, about 5-7, or about 6 to 7. In some embodiments, the N/P ratio may about 6, e.g., 6±1, or 6±0.5. In some embodiments, the N/P ratio may about 7, e.g., 7±1, or 7±0.5.

In some embodiments, the aqueous component comprises a biologically active agent. In some embodiments, the aqueous component comprises a polypeptide, optionally in combination with a nucleic acid. In some embodiments, the aqueous component comprises a nucleic acid, such as an RNA. In some embodiments, the aqueous component is a nucleic acid component. In some embodiments, the nucleic acid component comprises DNA and it can be called a DNA component. In some embodiments, the nucleic acid component comprises RNA. In some embodiments, the aqueous component, such as an RNA component may comprise an mRNA, such as an mRNA encoding an RNA-guided DNA-binding agent. In some embodiments, the RNA-guided DNA-binding agent is a Cas nuclease. In certain embodiments, aqueous component may comprise an mRNA that encodes a Cas nuclease, such as Cas9. In certain embodiments, the biologically active agent is a Cas nuclease mRNA. In certain embodiments, the biologically active agent is a Class 2 Cas nuclease mRNA. In certain embodiments, the biologically active agent is a Cas9 nuclease mRNA. In certain embodiments, the aqueous component may comprise a modified RNA. In some embodiments, the aqueous component may comprise a guide RNA nucleic acid. In certain embodiments, the aqueous component may comprise a gRNA. In certain embodiments, the aqueous component may comprise a dgRNA. In certain embodiments, the aqueous component may comprise a modified gRNA. In some compositions comprising an mRNA encoding an RNA-guided DNA-binding agent, the composition further comprises a gRNA nucleic acid, such as a gRNA. In some embodiments, the aqueous component comprises an RNA-guided DNA-binding agent and a gRNA. In some embodiments, the aqueous component comprises a Cas nuclease mRNA and a gRNA. In some embodiments, the aqueous component comprises a Class 2 Cas nuclease mRNA and a gRNA.

In certain embodiments, a lipid composition, such as an LNP composition, may comprise an mRNA encoding a Cas nuclease such as a Class 2 Cas nuclease, a compound of Formula (I) or (II) or a pharmaceutically acceptable salt thereof, a helper lipid, optionally a neutral lipid, and a PEG lipid. In certain compositions comprising an mRNA encoding a Cas nuclease such as a Class 2 Cas nuclease, the helper lipid is cholesterol. In other compositions comprising an mRNA encoding a Cas nuclease such as a Class 2 Cas nuclease, the neutral lipid is DSPC. In additional embodiments comprising an mRNA encoding a Cas nuclease such as a Class 2 Cas nuclease, e.g. Cas9, the PEG lipid is PEG2k-DMG. In specific compositions comprising an mRNA encoding a Cas nuclease such as a Class 2 Cas nuclease, and a compound of Formula (I) or (II) or a pharmaceutically acceptable salt thereof. In certain compositions, the composition further comprises a gRNA, such as a dgRNA or an sgRNA.

In some embodiments, a lipid composition, such as an LNP composition, may comprise a gRNA. In certain embodiments, a composition may comprise a compound of Formula (I) or (II) or a pharmaceutically acceptable salt thereof, a gRNA, a helper lipid, optionally a neutral lipid, and a PEG lipid. In certain LNP compositions comprising a gRNA, the helper lipid is cholesterol. In some compositions comprising a gRNA, the neutral lipid is DSPC. In additional embodiments comprising a gRNA, the PEG lipid is PEG2k-DMG. In certain compositions, the gRNA is selected from dgRNA and sgRNA.

In certain embodiments, a lipid composition, such as an LNP composition, comprises an mRNA encoding an RNA-guided DNA-binding agent and a gRNA, which may be an sgRNA, in an aqueous component and a compound of Formula (I) or (II) in a lipid component. For example, an LNP composition may comprise a compound of Formula (I) or (II) or a pharmaceutically acceptable salt thereof, an mRNA encoding a Cas nuclease, a gRNA, a helper lipid, a neutral lipid, and a PEG lipid. In certain compositions comprising an mRNA encoding a Cas nuclease and a gRNA, the helper lipid is cholesterol. In some compositions comprising an mRNA encoding a Cas nuclease and a gRNA, the neutral lipid is DSPC. In additional embodiments comprising an mRNA encoding a Cas nuclease and a gRNA, the PEG lipid is PEG2k-DMG.

In certain embodiments, the lipid compositions, such as LNP compositions include an RNA-guided DNA-binding agent, such as a Class 2 Cas mRNA and at least one gRNA. In some embodiments, the gRNA is a sgRNA. In some embodiments, the RNA-guided DNA-binding agent is a Cas9 mRNA In certain embodiments, the LNP composition includes a ratio of gRNA to RNA-guided DNA-binding agent mRNA, such as Class 2 Cas nuclease mRNA of about 1:1 or about 1:2. In some embodiments, the ratio of by weight is from about 25:1 to about 1:25, about 10:1 to about 1:10, about 8:1 to about 1:8, about 4:1 to about 1:4, about 2:1 to about 1:2, about 2:1 to 1:4 by weight, or about 1:1 to about 1:2.

The lipid compositions disclosed herein, such as LNP compositions, may be used in methods disclosed herein to deliver CRISPR/Cas9 components to insert a template nucleic acid, e.g., a DNA template. The template nucleic acid may be delivered separately from the lipid compositions comprising a compound of Formula (I) or (II) or a pharmaceutically acceptable salt thereof. In some embodiments, the template nucleic acid may be single- or double-stranded, depending on the desired repair mechanism. The template may have regions of homology to the target DNA, e.g. within the target DNA sequence, and/or to sequences adjacent to the target DNA.

In some embodiments, LNP compositions are formed by mixing an aqueous RNA solution with an organic solvent-based lipid solution. Suitable solutions or solvents include or may contain: water, PBS, Tris buffer, NaCl, citrate buffer, acetate buffer, ethanol, chloroform, diethylether, cyclohexane, tetrahydrofuran, methanol, isopropanol. For example, the organic solvent may be 100% ethanol. A pharmaceutically acceptable buffer, e.g., for in vivo administration of LNP compositions, may be used. In certain embodiments, a buffer is used to maintain the pH of the composition comprising LNPs at or above pH 6.5. In certain embodiments, a buffer is used to maintain the pH of the composition comprising LNPs at or above pH 7.0. In certain embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In certain embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In certain embodiments, the composition may comprise tris saline sucrose (TSS). In certain embodiments, the LNP composition may include about 1, 2, 3, 4, 5, 6, 7, 8, 9, or 10% cryoprotectant. In certain embodiments, the LNP composition may include about 1, 2, 3, 4, 5, 6, 7, 8, 9, or 10% sucrose. In some embodiments, the LNP composition may include a buffer. In some embodiments, the buffer may comprise a phosphate buffer (PBS), a Tris buffer, a citrate buffer, and mixtures thereof. In certain exemplary embodiments, the buffer comprises NaCl. In certain embodiments, the buffer lacks NaCl. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the LNP compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall composition is maintained. For example, the final osmolality may be maintained at less than 450 mOsm/L. In further embodiments, the osmolality is between 350 and 250 mOsm/L. Certain embodiments have a final osmolality of 300+/−20 mOsm/L or 310+/−40 mOsm/L.

In some embodiments, microfluidic mixing, T-mixing, or cross-mixing of the aqueous RNA solution and the lipid solution in an organic solvent is used. In certain aspects, flow rates, junction size, junction geometry, junction shape, tube diameter, solutions, and/or RNA and lipid concentrations may be varied. LNPs or LNP compositions may be concentrated or purified, e.g., via dialysis, centrifugal filter, tangential flow filtration, or chromatography. The LNP compositions may be stored as a suspension, an emulsion, or a lyophilized powder, for example. In some embodiments, an LNP composition is stored at 2-8° C., in certain aspects, the LNP compositions are stored at room temperature. In additional embodiments, an LNP composition is stored frozen, for example at −20° C. or −80° C. In other embodiments, an LNP composition is stored at a temperature ranging from about 0° C. to about −80° C. Frozen LNP compositions may be thawed before use, for example on ice, at room temperature, or at 25° C.

Preferred lipid compositions, such as LNP compositions, for example, are biodegradable, in that they do not accumulate to cytotoxic levels in vivo at a therapeutically effective dose. In some embodiments, the compositions do not cause an innate immune response that leads to substantial adverse effects at a therapeutic dose level. In some embodiments, the compositions provided herein do not cause toxicity at a therapeutic dose level.

In some embodiments, the concentration of the LNPs in the LNP composition is about 1-10 μg/mL, about 2-10 μg/mL, about 2.5-10 μg/mL, about 1-5 μg/mL, about 2-5 μg/mL, about 2.5-5 μg/mL, about 0.04 μg/mL, about 0.08 μg/mL, about 0.16 μg/mL, about 0.25 μg/mL, about 0.63 μg/mL, about 1.25 μg/mL, about 2.5 μg/mL, or about 5 μg/mL. In some embodiments, Dynamic Light Scattering (“DLS”) may be used to characterize the polydispersity index (PDI) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

In some embodiments, the LNPs disclosed herein have a PDI from about 0.005 to about 0.75. In some embodiments, the LNPs disclosed herein have a PDI from about 0.005 to about 0.1. In some embodiments, the LNPs disclosed herein have a PDI from about 0.005 to about 0.09, about 0.005 to about 0.08, about 0.005 to about 0.07, or about 0.006 to about 0.05. In some embodiments, the LNP have a PDI from about 0.01 to about 0.5. In some embodiments, the LNP have a PDI from about zero to about 0.4. In some embodiments, the LNP have a PDI from about zero to about 0.35. In some embodiments, the LNP PDI may range from about zero to about 0.3. In some embodiments, the LNP have a PDI that may range from about zero to about 0.25. In some embodiments, the LNP PDI may range from about zero to about 0.2. In some embodiments, the LNP have a PDI from about zero to about 0.05. In some embodiments, the LNP have a PDI from about zero to about 0.01. In some embodiments, the LNP have a PDI less than about 0.01, about 0.02, about 0.05, about 0.08, about 0.1, about 0.15, about 0.2, or about 0.4.

LNP size may be measured by various analytical methods known in the art. In some embodiments, LNP size may be measured using Asymetric-Flow Field Flow Fractionation—Multi-Angle Light Scattering (AF4-MALS). In certain embodiments, LNP size may be measured by separating particles in the composition by hydrodynamic radius, followed by measuring the molecular weights, hydrodynamic radii and root mean square radii of the fractionated particles. In some embodiments, LNP size and particle concentration may be measured by nanoparticle tracking analysis (NTA, Malvern Nanosight). In certain embodiments, LNP samples are diluted appropriately and injected onto a microscope slide. A camera records the scattered light as the particles are slowly infused through field of view. After the movie is captured, the Nanoparticle Tracking Analysis processes the movie by tracking pixels and calculating a diffusion coefficient. This diffusion coefficient can be translated into the hydrodynamic radius of the particle. Such methods may also count the number of individual particles to give particle concentration. In some embodiments, LNP size, morphology, and structural characteristics may be determined by cryo-electron microscopy (“cryo-EM”).

The LNPs of the LNP compositions disclosed herein have a size (e.g. Z-average diameter or number-average diameter) of about 1 to about 250 nm. In some embodiments, the LNPs have a size of about 10 to about 200 nm. In further embodiments, the LNPs have a size of about 20 to about 150 nm. In some embodiments, the LNPs have a size of about 50 to about 150 nm or about 70 to 130 nm. In some embodiments, the LNPs have a size of about 50 to about 100 nm. In some embodiments, the LNPs have a size of about 50 to about 120 nm. In some embodiments, the LNPs have a size of about 60 to about 100 nm. In some embodiments, the LNPs have a size of about 75 to about 150 nm. In some embodiments, the LNPs have a size of about 75 to about 120 nm. In some embodiments, the LNPs have a size of about 75 to about 100 nm. In some embodiments, the LNPs have a size of about 40 to about 125 nm, about 40 to about 110 nm, about 40 to about 100 nm, about 40 to about 90 nm, about 40 to about 85 nm, about 40 to about 80 nm, about 40 to about 75 nm, about 40 to about 70 nm, about 40 to about 65 nm, about 50 to about 125 nm, about 50 to about 110 nm, about 50 to about 100 nm, about 50 to about 90 nm, about 50 to about 85 nm, about 50 to about 80 nm, about 50 to about 75 nm, about 50 to about 70 nm, about 50 to about 65 nm, about 55 to about 125 nm, about 55 to about 110 nm, about 55 to about 100 nm, about 55 to about 90 nm, about 55 to about 85 nm, about 55 to about 80 nm, about 55 to about 75 nm, about 55 to about 70 nm, about 55 to about 65 nm, about 60 to about 125 nm, about 60 to about 110 nm, about 60 to about 100 nm, about 60 to about 90 nm, about 60 to about 85 nm, about 60 to about 80 nm, about 60 to about 75 nm, about 60 to about 70 nm, about 60 to about 65 nm, about 65 to about 125 nm, about 65 to about 110 nm, about 65 to about 100 nm, about 65 to about 90 nm, about 65 to about 85 nm, about 65 to about 80 nm, about 65 to about 75 nm, about 65 to about 70 nm, about 70 to about 125 nm, about 70 to about 110 nm, about 70 to about 100 nm, about 70 to about 90 nm, about 70 to about 85 nm, about 70 to about 80 nm, or about 70 to about 75 nm. In some embodiments, the LNPs have a size of less than about 95 nm or less than about 90 nm. In some embodiments, the LNPs have a size of greater than about 45 nm or greater than about 50 nm. In some embodiments, the particle size is a Z-average particle size. In some embodiments, the particle size is a number-average particle size. In some embodiments, the particle size is the size of an individual LNP. Unless indicated otherwise, all sizes referred to herein are the average sizes (diameters) of the fully formed nanoparticles, as measured by dynamic light scattering on a Malvern Zetasizer or Wyatt NanoStar. The nanoparticle sample is diluted in phosphate buffered saline (PBS) so that the count rate is approximately 200-400 kcps.

In some embodiments, the LNP compositions are formed with an average encapsulation efficiency ranging from about 50% to about 100%. In some embodiments, the LNP compositions are formed with an average encapsulation efficiency ranging from about 50% to about 95%. In some embodiments, the LNP compositions are formed with an average encapsulation efficiency ranging from about 70% to about 90%. In some embodiments, the LNP compositions are formed with an average encapsulation efficiency ranging from about 90% to about 100%. In some embodiments, the LNP compositions are formed with an average encapsulation efficiency ranging from about 75% to about 95%. In some embodiments, the LNP compositions are formed with an average encapsulation efficiency ranging from about 90% to about 100%. In some embodiments, the LNP compositions are formed with an average encapsulation efficiency ranging from about 92% to about 100%. In some embodiments, the LNP compositions are formed with an average encapsulation efficiency ranging from about 95% to about 100%. In some embodiments, the LNP compositions are formed with an average encapsulation efficiency ranging from about 98% to about 100%. In some embodiments, the LNP compositions are formed with an average encapsulation efficiency ranging from about 99% to about 100%.

Cargo

The cargo delivered via an LNP composition described herein include a biologically active agent. The biologically active agent may be a nucleic acid, such as an mRNA or gRNA. In certain embodiments, the cargo is or comprises one or more biologically active agent, such as mRNA, gRNA, expression vector, RNA-guided DNA-binding agent, antibody (e.g., monoclonal, chimeric, humanized, nanobody, and fragments thereof etc.), cholesterol, hormone, peptide, protein, chemotherapeutic and other types of antineoplastic agent, low molecular weight drug, vitamin, co-factor, nucleoside, nucleotide, oligonucleotide, enzymatic nucleic acid, antisense nucleic acid, triplex forming oligonucleotide, antisense DNA or RNA composition, chimeric DNA:RNA composition, allozyme, aptamer, ribozyme, decoys and analogs thereof, plasmid and other types of vectors, and small nucleic acid molecule, RNAi agent, short interfering nucleic acid (siNA), short interfering RNA (siRNA), double-stranded RNA (dsRNA), micro-RNA (miRNA), short hairpin RNA (shRNA) and “self-replicating RNA” (encoding a replicase enzyme activity and capable of directing its own replication or amplification in vivo) molecules, peptide nucleic acid (PNA), a locked nucleic acid ribonucleotide (LNA), morpholino nucleotide, threose nucleic acid (TNA), glycol nucleic acid (GNA), sisiRNA (small internally segmented interfering RNA), and iRNA (asymmetrical interfering RNA). The above list of biologically active agents is exemplary only, and is not intended to be limiting. Such compounds may be purified or partially purified, and may be naturally occurring or synthetic, and may be chemically modified.

The cargo delivered via LNP composition may be an RNA, such as an mRNA molecule encoding a protein of interest. For example, an mRNA for expressing a protein such as green fluorescent protein (GFP), an RNA-guided DNA-binding agent, or a Cas nuclease is included. LNP compositions that include a Cas nuclease mRNA, for example a Class 2 Cas nuclease mRNA that allows for expression in a cell of a Class 2 Cas nuclease such as a Cas9 or Cpf1 (also referred to as Cas12a) protein are provided. Further, the cargo may contain one or more gRNAs or nucleic acids encoding gRNAs. A template nucleic acid, e.g., for repair or recombination, may also be included with the compositions or a template nucleic acid may be used in the methods described herein. In a sub-embodiment, the cargo comprises an mRNA that encodes a Streptococcus pyogenes Cas9, optionally and an S. pyogenes gRNA. In a further sub-embodiment, the cargo comprises an mRNA that encodes a Neisseria meningitidis Cas9, optionally and an Nme (Neisseria meningitidis) gRNA.

“mRNA” refers to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

Genome Editing Tools

In some embodiments, the LNP composition is a lipid nucleic acid assembly, also referred to as a lipid nucleic acid composition. In some embodiments, the lipid nucleic acid composition or LNP composition comprises a genome editing tool or a nucleic acid encoding the same. As used herein, the term “genome editing tool” (or “gene editing tool”) is any component of “genome editing system” (or “gene editing system”) necessary or helpful for producing an edit in the genome of a cell. In some embodiments, the present disclosure provides for methods of delivering genome editing tools of a genome editing system (for example a zinc finger nuclease system, a TALEN system, a meganuclease system or a CRISPR/Cas system) to a cell (or population of cells). Genome editing tools include, for example, nucleases capable of making single or double strand break in the DNA or RNA of a cell, e.g., in the genome of a cell. The genome editing tools, e.g. nucleases, may optionally modify the genome of a cell without cleaving the nucleic acid, or nickases. A genome editing nuclease or nickase may be encoded by an mRNA. Such nucleases include, for example, RNA-guided DNA binding agents, and CRISPR/Cas components. Genome editing tools include fusion proteins, including e.g., a nickase fused to an effector domain such as an editor domain. Genome editing tools include any item necessary or helpful for accomplishing the goal of a genome edit, such as, for example, guide RNA, sgRNA, dgRNA, donor nucleic acid, and the like.

Various suitable gene editing systems comprising genome editing tools for delivery with the lipid nucleic acid assembly compositions are described herein, including but not limited to the CRISPR/Cas system; zinc finger nuclease (ZFN) system; and the transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507(7491): 258-261), which also may have the potential for uses in genome editing and gene therapy.

In certain embodiments, the disclosed compositions comprise one or more DNA modifying agents, such as a DNA cutting agent. A variety of DNA modifying agents may be included in the LNP compositions described herein. For example, DNA modifying agents include nucleases (both sequence-specific and non-specific), topoisomerases, methylases, acetylases, chemicals, pharmaceuticals, and other agents. In some embodiments, proteins that bind to a given DNA sequence or set of sequences may be employed to induce DNA modification such as strand breakage. Proteins can either be modified by many means, such as incorporation of ¹²⁵I, the radioactive decay of which would cause strand breakage, or modifying cross-linking reagents such as 4-azidophenacylbromide which form a cross-link with DNA on exposure to UV-light. Such protein-DNA cross-links can subsequently be converted to a double-stranded DNA break by treatment with piperidine. Yet another approach to DNA modification involves antibodies raised against specific proteins bound at one or more DNA sites, such as transcription factors or architectural chromatin proteins, and used to isolate the DNA from nucleoprotein complexes.

In certain embodiments, the disclosed compositions comprise one or more DNA cutting agents. DNA cutting agents include technologies such as Zinc-Finger Nucleases (ZFN), Transcription Activator-Like Effector Nucleases (TALEN), mito-TALEN, and meganuclease systems. TALEN and ZFN technologies use a strategy of tethering endonuclease catalytic domains to modular DNA binding proteins for inducing targeted DNA double-stranded breaks (DSB) at specific genomic loci. Additional DNA cutting agents include small interfering RNA, micro RNA, anti-microRNA, antagonist, small hairpin RNA, and aptamers (RNA, DNA or peptide based (including affimers)).

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing or for genome editing in situ, a technique known as genome editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

In preferred embodiments, the disclosed compositions comprise an mRNA encoding an RNA-guided DNA-binding agent, such as a Cas nuclease. In particular embodiments, the disclosed compositions comprise an mRNA encoding a Class 2 Cas nuclease, such as S. pyogenes Cas9.

As used herein, an “RNA-guided DNA-binding agent” means a polypeptide or complex of polypeptides having RNA and DNA-binding activity, or a DNA-binding subunit of such a complex, wherein the DNA-binding activity is sequence-specific and depends on the sequence of the RNA. Exemplary RNA-guided DNA-binding agents include Cas cleavases/nickases and inactivated forms thereof (“dCas DNA-binding agents”). “Cas nuclease”, as used herein, encompasses Cas cleavases, Cas nickases, and dCas DNA-binding agents. Cas cleavases/nickases and dCas DNA-binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA-binding activity. Class 2 Cas nucleases include Class 2 Cas cleavases/nickases (e.g., H840A, D10A, or N863A variants), which further have RNA-guided DNA cleavases or nickase activity, and Class 2 dCas DNA-binding agents, in which cleavase/nickase activity is inactivated. Class 2 Cas nucleases that may be used with the LNP compositions described herein include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables 2 and 4. See, e.g., Makarova et al., Nat Rev Microbiol, 13(11): 722-36 (2015); Shmakov et al., Molecular Cell, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In other embodiments, the Cas nuclease is the Cas9 nuclease from Streptococcus thermophilus. In still other embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In other embodiments, the Cas nuclease is the Cpf1 nuclease from Acidaminococcus sp. In still other embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In some embodiments, the Cas nuclease is a Cpf1 nuclease from an Acidaminococcus or Lachnospiraceae.

Wild type Cas9 has two nuclease domains: RuvC and HNH. The RuvC domain cleaves the non-target DNA strand, and the HNH domain cleaves the target strand of DNA. In some embodiments, the Cas9 nuclease comprises more than one RuvC domain and/or more than one HNH domain. In some embodiments, the Cas9 nuclease is a wild type Cas9. In some embodiments, the Cas9 is capable of inducing a double strand break in target DNA. In other embodiments, the Cas nuclease may cleave dsDNA, it may cleave one strand of dsDNA, or it may not have DNA cleavase or nickase activity.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease or Cas nickase may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain. In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell Oct 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA binding agent comprises a APOBEC3 deaminase. In some embodiments, a APOBEC3 deaminase is a APOBEC3A (A3A). In some embodiments, the A3A is a human A3A. In some embodiments, the A3A is a wild-type A3A.

In some embodiments, the RNA-guided DNA binding agent comprises an editor. An exemplary editor is BC22n which comprises a H. sapiens APOBEC3A fused to S. pyogenes-D10A Cas9 nickase by an XTEN linker. In some embodiments the editor is provided with a uracil glycosylase inhibitor (“UGI”). In some embodiments, the editor is fused to the UGI. In some embodiments, the mRNA encoding the editor and an mRNA encoding the UGI are formulated together in an LNP. In other embodiments, the editor and UGI are provided in separate LNPs.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS).

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitinlike protein (UBL). Non-limiting examples of ubiquitin-like proteins include small ubiquitinlike modifier (SUMO), ubiquitin cross-reactive protein (UCRP, also known as interferonstimulated gene-15 (ISG15)), ubiquitin-related modifier-1 (URM1), neuronal-precursor-cellexpressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6xHis, 8xHis, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain such as an editor domain. When the RNA-guided DNA-binding agent is directed to its target sequence, e.g., when a Cas nuclease is directed to a target sequence by a gRNA, the effector domain such as an editor domain may modify or affect the target sequence. In some embodiments, the effector domain such as an editor domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA. In some embodiments, the DNA modification domain is a methylation domain, such as a demethylation or methyltransferase domain. In some embodiments, the effector domain is a DNA modification domain, such as a base-editing domain. In particular embodiments, the DNA modification domain is a nucleic acid editing domain that introduces a specific modification into the DNA, such as a deaminase domain. See, e.g., WO 2015/089406; US 2016/0304846. The nucleic acid editing domains, deaminase domains, and Cas9 variants described in WO 2015/089406 and U.S. 2016/0304846 each of which is hereby incorporated by reference in its entirety.

The nuclease may comprise at least one domain that interacts with a guide RNA (“gRNA”). Additionally, the nuclease may be directed to a target sequence by a gRNA. In Class 2 Cas nuclease systems, the gRNA interacts with the nuclease as well as the target sequence, such that it directs binding to the target sequence. In some embodiments, the gRNA provides the specificity for the targeted cleavage, and the nuclease may be universal and paired with different gRNAs to cleave different target sequences. Class 2 Cas nuclease may pair with a gRNA scaffold structure of the types, orthologs, and exemplary species listed above.

As used herein, “ribonucleoprotein” (RNP) or “RNP complex” refers to a gRNA together with an RNA-guided DNA-binding agent, such as a Cas nuclease, e.g., a Cas cleavase, Cas nickase, or dCas DNA-binding agent (e.g., Cas9). In some embodiments, the gRNA guides the RNA-guided DNA-binding agent such as Cas9 to a target sequence, and the gRNA hybridizes with and the agent binds to the target sequence; in cases where the agent is a cleavase or nickase, binding can be followed by cleaving or nicking.

In some embodiments of the present disclosure, the cargo for the LNP composition includes at least one gRNA comprising guide sequences that direct an RNA-guided DNA-binding agent, which can be a nuclease (e.g., a Cas nuclease such as Cas9), to a target DNA. The gRNA may guide the Cas nuclease or Class 2 Cas nuclease to a target sequence on a target nucleic acid molecule. In some embodiments, a gRNA binds with and provides specificity of cleavage by a Class 2 Cas nuclease. In some embodiments, the gRNA and the Cas nuclease may form a ribonucleoprotein (RNP), e.g., a CRISPR/Cas complex such as a CRISPR/Cas9 complex. In some embodiments, the CRISPR/Cas complex may be a Type-II CRISPR/Cas9 complex. In some embodiments, the CRISPR/Cas complex may be a Type-V CRISPR/Cas complex, such as a Cpf1/gRNA complex. Cas nucleases and cognate gRNAs may be paired. The gRNA scaffold structures that pair with each Class 2 Cas nuclease vary with the specific CRISPR/Cas system.

“Guide RNA”, “gRNA”, and simply “guide” are used herein interchangeably to refer to a cognate guide nucleic acid for an RNA-guided DNA-binding agent. Guide RNAs can include modified RNAs as described herein. A gRNA may be, for example, either a single guide RNA, or the combination of a crRNA and a trRNA (also known as tracrRNA). The crRNA and trRNA may be associated as a single RNA molecule (as a single guide RNA, sgRNA) or, for example, in two separate RNA strands (dual guide RNA, dgRNA). In some systems a gRNA may be a crRNA (also known as a CRISPR RNA). “Guide RNA” or “gRNA” refers to each type. The trRNA may be a naturally-occurring sequence, or a trRNA sequence with modifications or variations compared to naturally-occurring sequences.

In some embodiments, an mRNA encoding a RNA-guided DNA binding agent is formulated in a first LNP composition and a gRNA nucleic acid is formulated in a second LNP composition. In some embodiments, the first and second LNP compositions are administered simultaneously. In other embodiments, the first and second LNP compositions are administered sequentially. In some embodiments, the first and second LNP compositions are combined prior to the preincubation step. In other embodiments, the first and second LNP compositions are preincubated separately.

In some embodiments, the cargo may comprise a DNA molecule. In some embodiments, the nucleic acid may comprise a nucleotide sequence encoding a crRNA. In some embodiments, the nucleotide sequence encoding the crRNA comprises a targeting sequence flanked by all or a portion of a repeat sequence from a naturally-occurring CRISPR/Cas system. In some embodiments, the nucleic acid may comprise a nucleotide sequence encoding a tracr RNA. In certain embodiments, the crRNA and the tracr RNA may be encoded by two separate nucleic acids. In other embodiments, the crRNA and the tracr RNA may be encoded by a single nucleic acid. In some embodiments, the crRNA and the tracr RNA may be encoded by opposite strands of a single nucleic acid. In other embodiments, the crRNA and the tracr RNA may be encoded by the same strand of a single nucleic acid. In some embodiments, the gRNA nucleic acid encodes an sgRNA. In some embodiments, the gRNA nucleic acid encodes a Cas9 nuclease sgRNA. In come embodiments, the gRNA nucleic acid encodes a Cpf1 nuclease sgRNA.

The nucleotide sequence encoding the guide RNA may be operably linked to at least one transcriptional or regulatory control sequence, such as a promoter, a 3′ UTR, or a 5′ UTR. In one example, the promoter may be a tRNA promoter, e.g., tRNALys3, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters also include U6 and H1 promoters. In some embodiments, the nucleotide sequence encoding the guide RNA may be operably linked to a mouse or human U6 promoter. In some embodiments, the gRNA nucleic acid is a modified nucleic acid. In some embodiments, the gRNA nucleic acid includes a modified nucleoside or nucleotide. In some embodiments, the gRNA nucleic acid includes a 5′ end modification, for example a modified nucleoside or nucleotide to stabilize and prevent integration of the nucleic acid. In other embodiments, the gRNA nucleic acid comprises a double-stranded DNA having a 5′ end modification on each strand. In some embodiments, the gRNA nucleic acid includes an inverted dideoxy-T or an inverted abasic nucleoside or nucleotide as the 5′ end modification. In some embodiments, the gRNA nucleic acid includes a label such as biotin, desthiobiotin-TEG, digoxigenin, and fluorescent markers, including, for example, FAM, ROX, TAMRA, and AlexaFluor.

As used herein, a “guide sequence” refers to a sequence within a gRNA that is complementary to a target sequence and functions to direct a gRNA to a target sequence for binding and/or modification (e.g., cleavage) by an RNA-guided DNA-binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about or at least 75%, 80%, 85%, 90%, 95%, 96%, 97%, 98%, 99%, or 100%. In some embodiments, the guide sequence and the target region may be 100% complementary or identical over a region of at least 15, 16, 17, 18, 19, or 20 contiguous nucleotides. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

In certain embodiments, multiple LNP compositions may be used collaboratively and/or for separate purposes. In some embodiments, a cell may be contacted with first and second LNP compositions described herein. In some embodiments, the first and second LNP compositions each independently comprise one or more of an mRNA, a gRNA, and a gRNA nucleic acid. In some embodiments, the first and second LNP compositions are administered simultaneously. In some embodiments, the first and second LNP compositions are administered sequentially.

In some embodiments, a method of producing multiple genome edits in a cell is provided (sometimes referred to herein and elsewhere as “multiplexing” or “multiplex gene editing” or “multiplex genome editing”). The ability to engineer multiple attributes into a single cell depends on the ability to perform edits in multiple targeted genes efficiently, including knockouts and in locus insertions, while retaining viability and the desired cell phenotype. In some embodiments, the method comprises culturing a cell in vitro, contacting the cell with two or more lipid nucleic acid assembly compositions, wherein each lipid nucleic acid assembly composition comprises a nucleic acid genome editing tool capable of editing a target site, and expanding the cell in vitro. The method results in a cell having more than one genome edit, wherein the genome edits differ. In certain embodiments, the first LNP composition comprises a first gRNA and the second LNP composition comprises a second gRNA, wherein the first and second gRNAs comprise different guide sequences that are complementary to different targets. In such embodiments, the LNP compositions may allow for multiplex gene editing. In some embodiments, the cell is contacted with 1, 2, 3, 4, 5, 6, 7, 8, 9, 10, or 11 lipid nucleic acid assembly compositions. In some embodiments, the cell is contacted with at least 6 lipid nucleic acid assembly compositions.

Target sequences for RNA-guided DNA-binding proteins such as Cas proteins include both the positive and negative strands of genomic DNA (i.e., the sequence given and the sequence's reverse complement), as a nucleic acid substrate for a Cas protein is a double stranded nucleic acid. Accordingly, where a guide sequence is said to be “complementary to a target sequence”, it is to be understood that the guide sequence may direct a gRNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

In some embodiments, gRNA described herein targets a gene that reduces or eliminates surface expression of a T cell receptor, MEW class I, or MEW class II. In certain embodiments, gRNA described herein targets TRAC. In some embodiments, gRNA described herein targets TRBC. In further embodiments, gRNA described herein targets CIITA. In other embodiments, gRNA described herein targets HLA-A. In some embodiments, gRNA described herein targets HLA-B. In other embodiments, gRNA described herein targets HLA-C. In further embodiments, gRNA described herein targets B2M. In some embodiments, methods are provided for producing multiple genome edits in an vitro-cultured cell, comprising the steps of: a) contacting the cell in vitro with at least a first lipid composition comprising a first nucleic acid, thereby producing a contacted cell; b) contacting the cell in vitro with at least a second lipid composition comprising a second nucleic acid, wherein the second nucleic acid is different from the first nucleic acid; and c) expanding the cell in vitro. In certain embodiments, methods are provided for producing multiple genome edits in an in vitro-cultured cell, comprising the steps of: a) contacting the cell in vitro with at least a first lipid composition comprising a first nucleic acid, thereby producing a contacted cell; b) culturing the contacted cell in vitro, thereby producing a cultured contacted cell; c) contacting the cultured contacted cell in vitro with at least a second lipid composition comprising a second nucleic acid, wherein the second nucleic acid is different from the first nucleic acid; and d) expanding the cell in vitro. In further embodiments, the methods further comprise contacting the cell in vitro with at least a third lipid composition comprising a third nucleic acid, wherein the third nucleic acid is different from the first and second nucleic acids. In still further embodiments, the methods further comprise contacting the cell in vitro with at least a fourth lipid composition comprising a fourth nucleic acid, wherein the fourth nucleic acid is different from the first second, and third nucleic acids. In still yet further embodiments, the methods further comprise contacting the cell in vitro with at least a fifth lipid composition comprising a fifth nucleic acid, wherein the fifth nucleic acid is different from the first second, third, and fourth nucleic acids. In additional embodiments, the methods further comprise contacting the cell in vitro with at least a sixth lipid composition comprising a sixth nucleic acid, wherein the sixth nucleic acid is different from the first second, third, fourth, and fifth nucleic acids. In certain embodiments, at least two of the lipid compositions are administered sequentially. In some embodiments, at least two of the lipid compositions are administered simultaneously. In some embodiments, the expanded cell exhibits increased survival.

In some embodiments, the nucleic acid of any of the foregoing methods for producing multiple genome edits in an in vitro-cultured cell is an RNA, such as a gRNA. In certain embodiments, at least one of the lipid compositions comprises a gRNA targeting TRAC. In some embodiments, at least one of the lipid compositions comprises a gRNA targeting TRBC. In further embodiments, at least one of the lipid compositions comprises a gRNA targeting a gene that reduces or eliminates surface expression of MHC class I. In still further embodiments, at least one of the lipid compositions comprises a gRNA targeting a gene that reduces or eliminates surface expression of MHC class II. In certain embodiments, at least one of the lipid compositions comprises a gRNA targeting TRAC, and at least one of the lipid compositions comprises a gRNA targeting TRBC. In some embodiments, at least one of the lipid compositions comprises a gRNA targeting HLA-A, optionally wherein the cell is homozygous for HLA-B and homozygous for HLA-C. In some embodiments, at least one of the lipid compositions comprises a gRNA targeting CIITA. In certain embodiments, at least one of the lipid compositions comprises a gRNA targeting TRAC, at least one of the lipid compositions comprises a gRNA targeting TRBC, at least one of the lipid compositions comprises a gRNA targeting HLA-A, and at least one of the lipid compositions comprises a gRNA targeting CIITA. In certain embodiments, at least one of the lipid compositions comprises a gRNA targeting TRAC, at least one of the lipid compositions comprises a gRNA targeting TRBC, a gRNA targeting HLA-A, and at least one of the lipid compositions comprises a gRNA that reduces or eliminates surface expression of MHC class II. In certain embodiments, at least one of the lipid compositions comprises a gRNA targeting TRAC, at least one of the lipid compositions comprises a gRNA targeting TRBC, at least one of the lipid compositions comprises a gRNA targeting a gene that reduces or eliminates surface expression of MHC class I, and at least one of the lipid compositions comprises a gRNA targeting CIITA. In certain embodiments, at least one of the lipid compositions comprises a gRNA targeting a gene that reduces or eliminates surface expression of a T cell receptor, at least one of the lipid compositions comprises a gRNA targeting HLA-A, and at least one of the lipid compositions comprises a gRNA targeting CIITA. In certain embodiments, at least one of the lipid compositions comprises a gRNA targeting TRAC, at least one of the lipid compositions comprises a gRNA targeting HLA-A, and at least one of the lipid compositions comprises a gRNA targeting CIITA. In certain embodiments, at least one of the lipid compositions comprises a gRNA targeting a gene that reduces or eliminates surface expression of MHC class I, and at least one of the lipid compositions comprises a gRNA targeting CIITA.

In certain embodiments, at least one of the foregoing lipid compositions comprises a nucleic acid genome editing tool as described herein. In some embodiments, a further lipid composition comprises an RNA-guided DNA binding agent. In some embodiments, the RNA-guided DNA binding agent is Cas9.

In some embodiments, the methods of the present disclosure further comprise contacting the cell with a donor nucleic acid. In some embodiments, a further lipid composition comprises a donor nucleic acid. The donor nucleic acid may be inserted in a target sequence. In some embodiments, a donor nucleic acid sequence is provided as a vector. In some embodiments, the donor nucleic acid encodes a targeting receptor. In certain embodiments, the donor nucleic acid comprises regions having homology with corresponding regions of a T cell receptor sequence. A “targeting receptor” is a polypeptide present on the surface of a cell, e.g., a T cell, to permit binding of the cell to a target site, e.g., a specific cell or tissue in an organism. In some embodiments, the targeting receptor is a CAR. In some embodiments, the targeting receptor is a universal CAR (UniCAR). In some embodiments, the targeting receptor is a TCR. In some embodiments, the targeting receptor is a T cell receptor fusion construct (TRuC). In some embodiments, the targeting receptor is a B cell receptor (BCR) (e.g., expressed on a B cell). In some embodiments, the targeting receptor is chemokine receptor. In some embodiments, the targeting receptor is a cytokine receptor.

In some embodiments, the in vitro genome editing methods have produced high editing efficiency at multiple target sites in T cells. In some embodiments, an engineered T cell is produced wherein the endogenous TCR is knocked out. In some embodiments, an engineered T cell is produced wherein expression of the endogenous TCR is knocked out. In some embodiments, an engineered T cell is produced wherein two genes have reduced expression and/or are knocked out. In some embodiments, an engineered T cell is produced wherein three genes are knocked down and/or are knocked out. In some embodiments, an engineered T cell is produced wherein four genes are knocked down and/or are knocked out. In some embodiments, an engineered T cell is produced wherein five genes are knocked down and/or are knocked out. In some embodiments, an engineered T cell is produced wherein six genes are knocked down and/or are knocked out. In some embodiments, an engineered T cell is produced wherein seven genes are knocked down and/or are knocked out. In some embodiments, an engineered T cell is produced wherein eight genes are knocked down and/or are knocked out. In some embodiments, an engineered T cell is produced wherein nine genes are knocked down and/or are knocked out. In some embodiments, an engineered T cell is produced wherein ten genes have are knocked down and/or are knocked out. In some embodiments, an engineered T cell is produced wherein eleven genes are knocked down and/or are knocked out.

In some embodiments, an engineered T cell is produced wherein the endogenous TCR is knocked out and a transgenic TCR is inserted and expressed. In some embodiments, the engineered T cell is a primary human T cell. In some embodiments, the tgTCR targets Wilms' Tumor 1 (WT1). In some embodiments, the WT1 tgTCR is inserted into a high proportion of T cells (e.g., greater than 55%, 60%, 65%, 70%, 75%, 80%, 85%, 90%, or 95%) using the disclosed lipid composition.

β2M or β2M are used interchangeably herein and with reference to nucleic acid sequence or protein sequence of β-2 microglobulin; the human gene has accession number NC_000015 (range 44711492 . . . 44718877), reference GRCh38.p13. The B2M protein is associated with MHC class I molecules as a heterodimer on the surface of nucleated cells and is required for MHC class I protein expression.

CIITA or CIITA or C2TA are used interchangeably herein and with reference to the nucleic acid sequence or protein sequence of class II major histocompatibility complex transactivator; the human gene has accession number NC_000016.10 (range 10866208 . . . 10941562), reference GRCh38.p13, incorporated by referenced herein. The CIITA protein in the nucleus acts as a positive regulator of MHC class II gene transcription and is required for MHC class II protein expression.

MHC or MHC molecule(s) or MHC protein or MHC complex(es), refer to a major histocompatibility complex molecule (or plural), and include e.g., MHC class I and MHC class II molecules. In humans, MHC molecules are referred to as human leukocyte antigen complexes or HLA molecules or HLA protein. The use of terms MHC and HLA are not meant to be limiting; as used herein, the term MHC may be used to refer to human MHC molecules, i.e., HLA molecules. Therefore, the terms MHC and HLA are used interchangeably herein.

HLA-A as used herein in the context of HLA-A protein, refers to the MHC class I protein molecule, which is a heterodimer consisting of a heavy chain (encoded by the HLA-A gene) and a light chain (i.e., beta-2 microglobulin). The terms HLA-A or HLA-A gene, as used herein in the context of nucleic acids refers to the gene encoding the heavy chain of the HLA-A protein molecule. The HLA-A gene is also referred to as HLA class I histocompatibility, A alpha chain; the human gene has accession number NC_000006.12 (29942532 . . . 29945870), incorporated by referenced herein. The HLA-A gene is known to have hundreds of different versions (also referred to as alleles) across the population (and an individual may receive two different alleles of the HLA-A gene). All alleles of HLA-A are encompassed by the terms HLA-A and HLA-A gene.

HLA-B as used herein in the context of nucleic acids refers to the gene encoding the heavy chain of the HLA-B protein molecule. The HLA-B is also referred to as HLA class I histocompatibility, B alpha chain; the human gene has accession number NC_000006.12 (31353875 . . . 31357179), incorporated by referenced herein.

HLA-C as used herein in the context of nucleic acids refers to the gene encoding the heavy chain of the HLA-C protein molecule. The HLA-C is also referred to as HLA class I histocompatibility, C alpha chain; the human gene has accession number NC_000006.12 (31268749 . . . 31272092), incorporated by referenced herein.

The length of the targeting sequence may depend on the CRISPR/Cas system and components used. For example, different Class 2 Cas nucleases from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence length is 0, 1, 2, 3, 4, or 5 nucleotides longer or shorter than the guide sequence of a naturally-occurring CRISPR/Cas system. In certain embodiments, the Cas nuclease and gRNA scaffold will be derived from the same CRISPR/Cas system. In some embodiments, the targeting sequence may comprise or consist of 18-24 nucleotides. In some embodiments, the targeting sequence may comprise or consist of 19-21 nucleotides. In some embodiments, the targeting sequence may comprise or consist of 20 nucleotides.

In some embodiments, the sgRNA is a “Cas9 sgRNA” capable of mediating RNA-guided DNA cleavage by a Cas9 protein. In some embodiments, the sgRNA is a “Cpf1 sgRNA” capable of mediating RNA-guided DNA cleavage by a Cpf1 protein. In certain embodiments, the gRNA comprises a crRNA and tracr RNA sufficient for forming an active complex with a Cas9 protein and mediating RNA-guided DNA cleavage. In certain embodiments, the gRNA comprises a crRNA sufficient for forming an active complex with a Cpf1 protein and mediating RNA-guided DNA cleavage. See Zetsche 2015.

Certain embodiments also provide nucleic acids, e.g., expression cassettes, encoding the gRNA described herein. A “guide RNA nucleic acid” is used herein to refer to a gRNA (e.g. an sgRNA or a dgRNA) and a gRNA expression cassette, which is a nucleic acid that encodes one or more gRNAs.

### Modified RNAs

In certain embodiments, the lipid compositions, such as LNP compositions comprise modified nucleic acids, including modified RNAs.

Modified nucleosides or nucleotides can be present in an RNA, for example a gRNA or mRNA. A gRNA or mRNA comprising one or more modified nucleosides or nucleotides, for example, is called a “modified” RNA to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide, here called “modified.”

Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the polynucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Certain embodiments comprise a 5′ end modification to an mRNA, gRNA, or nucleic acid. Certain embodiments comprise a modification to an mRNA, gRNA, or nucleic acid. Certain embodiments comprise a 3′ end modification to an mRNA, gRNA, or nucleic acid. A modified RNA can contain 5′ end and 3′ end modifications. A modified RNA can contain one or more modified residues at non-terminal locations. In certain embodiments, a gRNA includes at least one modified residue. In certain embodiments, an mRNA includes at least one modified residue. In certain embodiments, the modified gRNA comprises a modification at one or more of the first five nucleotides at a 5′ end. In certain embodiments, the modified gRNA comprises a modification at one or more of the first five nucleotides at a 5′ end. The LNP composition of claim 52 or 53, wherein the modified gRNA comprises a modification at one or more of the last five nucleotides at a 3′ end.

Unmodified nucleic acids can be prone to degradation by, e.g., intracellular nucleases or those found in serum. For example, nucleases can hydrolyze nucleic acid phosphodiester bonds. Accordingly, in one aspect the RNAs (e.g. mRNAs, gRNAs) described herein can contain one or more modified nucleosides or nucleotides, e.g., to introduce stability toward intracellular or serum-based nucleases. In some embodiments, the modified RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

Accordingly, in some embodiments, an RNA or nucleic acid comprises at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the RNA or nucleic acid more stable (e.g., resistant to nuclease digestion) than the wild-type or naturally occurring version of the RNA or nucleic acid. As used herein, the terms “stable” and “stability” and such terms relate to the nucleic acids described herein, and particularly with respect to the RNA, refer to increased or enhanced resistance to degradation by, for example nucleases (i.e., endonucleases or exonucleases) which are normally capable of degrading such RNA. Increased stability can include, for example, less sensitivity to hydrolysis or other destruction by endogenous enzymes (e.g., endonucleases or exonucleases) or conditions within the target cell or tissue, thereby increasing or enhancing the residence of such RNA or nucleic acid in the target cell, tissue, subject and/or cytoplasm. The stabilized RNA or nucleic acid molecules provided herein demonstrate longer half-lives relative to their naturally occurring, unmodified counterparts (e.g. the wild-type version of the molecule). Also contemplated by the terms “modification” and “modified” as such terms related to the mRNA of the LNP compositions disclosed herein are alterations which improve or enhance translation of mRNA nucleic acids, including for example, the inclusion of sequences which function in the initiation of protein translation (e.g., the Kozak consensus sequence). (Kozak, M., Nucleic Acids Res 15 (20): 8125-48 (1987)).

In some embodiments, the RNA or nucleic acid has undergone a chemical or biological modification to render it more stable. Exemplary modifications to an RNA or nucleic acid include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring RNA or nucleic acids, for example, covalent modifications such as the introduction of modified nucleotides, (e.g., nucleotide analogs, or the inclusion of pendant groups which are not naturally found in such RNA, such as a deoxynucleoside, or nucleic acid molecules).

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens. The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

mRNAs

In some embodiments, a composition or formulation disclosed herein comprises an mRNA comprising an open reading frame (ORF) encoding an RNA-guided DNA-binding agent, such as a Cas nuclease, or Class 2 Cas nuclease as described herein. In some embodiments, an mRNA comprising an ORF encoding an RNA-guided DNA-binding agent, such as a Cas nuclease or Class 2 Cas nuclease, is provided, used, or administered. An mRNA may comprise one or more of a 5′ cap, a 5′ untranslated region (UTR), a 3′ UTRs, and a polyadenine tail. The mRNA may comprise a modified open reading frame, for example to encode a nuclear localization sequence or to use alternate codons to encode the protein.

The mRNA in the disclosed LNP compositions may encode a cell surface or intracellular polypeptide. The mRNA in the disclosed LNP compositions may encode, for example, a secreted hormone, enzyme, receptor, polypeptide, peptide or other protein of interest that is normally secreted. In some embodiments, the mRNA may optionally have chemical or biological modifications which, for example, improve the stability and/or half-life of such mRNA or which improve or otherwise facilitate protein production.

In addition, suitable modifications include alterations in one or more nucleotides of a codon such that the codon encodes the same amino acid but is more stable than the codon found in the wild-type version of the mRNA. For example, an inverse relationship between the stability of RNA and a higher number cytidines (C's) and/or uridines (U's) residues has been demonstrated, and RNA devoid of C and U residues have been found to be stable to most RNases (Heidenreich, et al. J Biol Chem 269, 2131-8 (1994)). In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids also include the incorporation of pseudouridines. The incorporation of pseudouridines into the mRNA nucleic acids may enhance stability and translational capacity, as well as diminishing immunogenicity in vivo. See, e.g., Karikó, K., et al., Molecular Therapy 16 (11): 1833-1840 (2008). Substitutions and modifications to the mRNA may be performed by methods readily known to one or ordinary skill in the art.

The constraints on reducing the number of C and U residues in a sequence will likely be greater within the coding region of an mRNA, compared to an untranslated region, (i.e., it will likely not be possible to eliminate all of the C and U residues present in the message while still retaining the ability of the message to encode the desired amino acid sequence). The degeneracy of the genetic code, however presents an opportunity to allow the number of C and/or U residues that are present in the sequence to be reduced, while maintaining the same coding capacity (i.e., depending on which amino acid is encoded by a codon, several different possibilities for modification of RNA sequences may be possible).

The term modification also includes, for example, the incorporation of non-nucleotide linkages or modified nucleotides into the mRNA sequences (e.g., modifications to one or both the 3′ and 5′ ends of an mRNA molecule encoding a functional secreted protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements which change the structure of an mRNA molecule (e.g., which form secondary structures).

The poly A tail is thought to stabilize natural messengers. Therefore, a long poly A tail may be added to an mRNA molecule thus rendering the mRNA more stable. Poly A tails can be added using a variety of art-recognized techniques. For example, long poly A tails can be added to synthetic or in vitro transcribed mRNA using poly A polymerase (Yokoe, et al. Nature Biotechnology. 1996; 14: 1252-1256). A transcription vector can also encode long poly A tails. In addition, poly A tails can be added by transcription directly from PCR products. In some embodiments, the length of the poly A tail is at least about 90, 200, 300, 400 at least 500 nucleotides. In certain embodiments, the length of the poly A tail is adjusted to control the stability of a modified mRNA molecule and, thus, the transcription of protein. For example, since the length of the poly A tail can influence the half-life of an mRNA molecule, the length of the poly A tail can be adjusted to modify the level of resistance of the mRNA to nucleases and thereby control the time course of protein expression in a cell. In some embodiments, the stabilized mRNA molecules are sufficiently resistant to in vivo degradation (e.g., by nucleases), such that they may be delivered to the target cell without a transfer vehicle.

In certain embodiments, an mRNA can be modified by the incorporation 3′ and/or 5′ untranslated (UTR) sequences which are not naturally found in the wild-type mRNA. In some embodiments, 3′ and/or 5′ flanking sequence which naturally flanks an mRNA and encodes a second, unrelated protein can be incorporated into the nucleotide sequence of an mRNA molecule encoding a therapeutic or functional protein in order to modify it. For example, 3′ or 5′ sequences from mRNA molecules which are stable (e.g., globin, actin, GAPDH, tubulin, histone, or citric acid cycle enzymes) can be incorporated into the 3′ and/or 5′ region of a sense mRNA nucleic acid molecule to increase the stability of the sense mRNA molecule. See, e.g., US2003/0083272.

More detailed descriptions of the mRNA modifications can be found in US2017/0210698A1, at pages 57-68, the contents of which are incorporated herein.

Template Nucleic Acid

The methods disclosed herein may include using a template nucleic acid. The template may be used to alter or insert a nucleic acid sequence at or near a target site for an RNA-guided DNA-binding protein such as a Cas nuclease, e.g., a Class 2 Cas nuclease. In some embodiments, the methods comprise introducing a template to the cell. In some embodiments, a single template may be provided. In other embodiments, two or more templates may be provided such that editing may occur at two or more target sites. For example, different templates may be provided to edit a single gene in a cell, or two different genes in a cell.

In some embodiments, the template may be used in homologous recombination. In some embodiments, the homologous recombination may result in the integration of the template sequence or a portion of the template sequence into the target nucleic acid molecule. In other embodiments, the template may be used in homology-directed repair, which involves DNA strand invasion at the site of the cleavage in the nucleic acid. In some embodiments, the homology-directed repair may result in including the template sequence in the edited target nucleic acid molecule. In yet other embodiments, the template may be used in gene editing mediated by non-homologous end joining. In some embodiments, the template sequence has no similarity to the nucleic acid sequence near the cleavage site. In some embodiments, the template or a portion of the template sequence is incorporated. In some embodiments, the template includes flanking inverted terminal repeat (ITR) sequences.

In some embodiments, the template sequence may correspond to, comprise, or consist of an endogenous sequence of a target cell. It may also or alternatively correspond to, comprise, or consist of an exogenous sequence of a target cell. As used herein, the term “endogenous sequence” refers to a sequence that is native to the cell. The term “exogenous sequence” refers to a sequence that is not native to a cell, or a sequence whose native location in the genome of the cell is in a different location. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell.

In some embodiments, the template contains ssDNA or dsDNA containing flanking invert-terminal repeat (ITR) sequences. In some embodiments, the template is provided as a vector, plasmid, minicircle, nanocircle, or PCR product.

In some embodiments, the nucleic acid is purified. In some embodiments, the nucleic acid is purified using a precipitation method (e.g., LiCl precipitation, alcohol precipitation, or an equivalent method, e.g., as described herein). In some embodiments, the nucleic acid is purified using a chromatography-based method, such as an HPLC-based method or an equivalent method (e.g., as described herein). In some embodiments, the nucleic acid is purified using both a precipitation method (e.g., LiCl precipitation) and an HPLC-based method. In some embodiments, the nucleic acid is purified by tangential flow filtration (TFF).

Cell Types

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3+, CD4+ and CD8+ T cells, regulatory T cells (Tregs), B cells, NK cells, and dendritic cells (DC). In some embodiments, the immune cell is allogeneic. In some embodiments, the cell is a lymphocyte. In some embodiments, the cell is an adaptive immune cell. In some embodiments, the cell is a T cell. In some embodiments, the cell is a B cell. In some embodiments, the cell is a NK cell.

As used herein, a T cell can be defined as a cell that expresses a T cell receptor (“TCR” or “αβ TCR” or “γδ TCR”), however in some embodiments, the TCR of a T cell may be genetically modified to reduce its expression (e.g., by genetic modification to the TRAC or TRBC genes), therefore expression of the protein CD3 may be used as a marker to identify a T cell by standard flow cytometry methods. CD3 is a multi-subunit signaling complex that associates with the TCR. Thus, a T cell may be referred to as CD3+. In some embodiments, a T cell is a cell that expresses a CD3+ marker and either a CD4+ or CD8+ marker.

In some embodiments, the T cell expresses the glycoprotein CD8 and therefore is CD8+ by standard flow cytometry methods and may be referred to as a “cytotoxic” T cell. In some embodiments, the T cell expresses the glycoprotein CD4 and therefore is CD4+ by standard flow cytometry methods and may be referred to as a “helper” T cell. CD4+ T cells can differentiate into subsets and may be referred to as a Th1 cell, Th2 cell, Th9 cell, Th17 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T cell may be isolated from a subject by CD4+ or CD8+ selection methods.

In some embodiments, the T cell is a memory T cell. In the body, a memory T cell has encountered antigen. A memory T cell can be located in the secondary lymphoid organs (central memory T cells) or in recently infected tissue (effector memory T cells). A memory T cell may be a CD8+ T cell. A memory T cell may be a CD4+ T cell. As used herein, a “central memory T cell” can be defined as an antigen-experienced T cell, and for example, may expresses CD62L and CD45RO. A central memory T cell may be detected as CD62L+ and CD45RO+ by Central memory T cells also express CCR7, therefore may be detected as CCR7+ by standard flow cytometry methods.

As used herein, an “early stem-cell memory T cell” (or “Tscm”) can be defined as a T cell that expresses CD27 and CD45RA, and therefore is CD27+ and CD45RA+ by standard flow cytometry methods. A Tscm does not express the CD45 isoform CD45RO, therefore a Tscm will further be CD45RO− if stained for this isoform by standard flow cytometry methods. A CD45RO− CD27+ cell is therefore also an early stem-cell memory T cell. Tscm cells further express CD62L and CCR7, therefore may be detected as CD62L+ and CCR7+ by standard flow cytometry methods. Early stem-cell memory T cells have been shown to correlate with increased persistence and therapeutic efficacy of cell therapy products.

In some embodiments, the cell is a B cell. As used herein, a “B cell” can be defined as a cell that expresses CD19 and/or CD20, and/or B cell mature antigen (“BCMA”), and therefore a B cell is CD19+, and/or CD20+, and/or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+ or has a class-switched B cell receptor (e.g., IgG+, or IgA+).

Cells used in ACT therapy are included, such as mesenchymal stem cells (e.g., isolated from bone marrow (BM), peripheral blood (PB), placenta, umbilical cord (UC) or adipose); hematopoietic stem cells (HSCs; e.g. isolated from BM); mononuclear cells (e.g., isolated from BM or PB); endothelial progenitor cells (EPCs; isolated from BM, PB, and UC); neural stem cells (NSCs); limbal stem cells (LSCs); or tissue-specific primary cells or cells derived therefrom (TSCs). Cells used in ACT therapy further include induced pluripotent stem cells (iPSCs) that may be induced to differentiate into other cell types including e.g., islet cells, neurons, and blood cells; ocular stem cells; pluripotent stem cells (PSCs); embryonic stem cells (ESCs); cells for organ or tissue transplantations such as islet cells, cardiomyocytes, thyroid cells, thymocytes, neuronal cells, skin cells, retinal cells, chondrocytes, myocytes, and keratinocytes.

In some embodiments, the cell is a human cell, such as a cell from a subject. In some embodiments, the cell is isolated from a human subject, such as a human donor. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”).

In some embodiments, the methods are carried out ex vivo. As used herein, “ex vivo” refers to an in vitro method wherein the cell is capable of being transferred into a subject, e.g., as an ACT therapy. In some embodiments, an ex vivo method is an in vitro method involving an ACT therapy cell or cell population.

In some embodiments, the cell is maintained in culture. In some embodiments, the cell is transplanted into a patient. In some embodiments, the cell is removed from a subject, genetically modified ex vivo, and then administered back to the same patient. In some embodiments, the cell is removed from a subject, genetically modified ex vivo, and then administered to a subject other than the subject from which it was removed.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

In some embodiments, the T cell is activated by polyclonal activation (or “polyclonal stimulation”) (not antigen-specific stimulation). In some embodiments, the T cell is activated by CD3 stimulation (e.g., providing an anti-CD3 antibody). In some embodiments, the T cell is activated by CD3 and CD28 stimulation (e.g., providing an anti-CD3 antibody and an anti-CD28 antibody). In some embodiments, the T cell is activated using a ready-to-use reagent to activate the T cell (e.g., via CD3/CD28 stimulation). In some embodiments, the T cell is activated by via CD3/CD28 stimulation provided by beads. In some embodiments, the T cell is activated by via CD3/CD28 stimulation wherein one or more components is soluble and/or one or more components is bound to a solid surface (e.g., plate or bead). In some embodiments, the T cell is activated by an antigen-independent mitogen (e.g., a lectin, including e.g., concanavalin A (“ConA”), or PHA).

In some embodiments, one or more cytokines are used for activation of T cells. IL-2 is provided for T cell activation. In some embodiments, the cytokine(s) for activation of T cells is a cytokine that binds to the common gamma chain (γc) receptor. In some embodiments, IL-2 is provided for T cell activation and/or to promote T cell survival. In some embodiments, IL-7 is provided for T cell activation. In some embodiments, IL-15 is provided for T cell activation. In some embodiments, IL-21 is provided for T cell activation. In some embodiments, a combination of cytokines is provided for T cell activation, including, e.g., IL-2, IL-7, IL-15, and/or IL-21.

In some embodiments, the T cell is activated by exposing the cell to an antigen (antigen stimulation). A T cell is activated by antigen when the antigen is presented as a peptide in a major histocompatibility complex (“MHC”) molecule (peptide-MHC complex). A cognate antigen may be presented to the T cell by co-culturing the T cell with an antigen-presenting cell (feeder cell) and antigen. In some embodiments, the T cell is activated by co-culture with an antigen-presenting cell that has been pulsed with antigen. In some embodiments, the antigen-presenting cell has been pulsed with a peptide of the antigen.

In some embodiments, the T cell may be activated for 12 to 72 hours. In some embodiments, the T cell may be activated for 12 to 48 hours. In some embodiments, the T cell may be activated for 12 to 24 hours. In some embodiments, the T cell may be activated for 24 to 48 hours. In some embodiments, the T cell may be activated for 24 to 72 hours. In some embodiments, the T cell may be activated for 12 hours. In some embodiments, the T cell may be activated for 48 hours. In some embodiments, the T cell may be activated for 72 hours.

While the inventions are described in conjunction with the illustrated embodiments, it is understood that they are not intended to limit the invention to those embodiments. On the contrary, the disclosure is intended to cover all alternatives, modifications, and equivalents, including equivalents of specific features, which may be included within the inventions as defined by the appended claims.

Both the foregoing general description and detailed description, as well as the following examples, are exemplary and explanatory only and are not restrictive of the teachings. The section headings used herein are for organizational purposes only and are not to be construed as limiting the desired subject matter in any way. In the event that any literature incorporated by reference contradicts any term defined in this specification, this specification controls. All ranges given in the application encompass the endpoints unless stated otherwise.

### Definitions

It should be noted that, as used in this application, the singular form “a”, “an” and “the” include plural references unless the context clearly dictates otherwise. Thus, for example, reference to “a composition” includes a plurality of compositions and reference to “a cell” includes a plurality of cells and the like. The use of “or” is inclusive and means “and/or” unless stated otherwise.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; embodiments in the specification that recite “about” various components are also contemplated as “at” the recited components; and embodiments in the specification that recite “consisting essentially of” various components are also contemplated as “consisting of” or “comprising” the recited components (this interchangeability does not apply to the use of these terms in the claims).

Numeric ranges are inclusive of the numbers defining the range. Measured and measurable values are understood to be approximate, taking into account significant digits and the error associated with the measurement. As used in this application, the terms “about” and “approximately” have their art-understood meanings; use of one vs the other does not necessarily imply different scope. Unless otherwise indicated, numerals used in this application, with or without a modifying term such as “about” or “approximately”, should be understood to encompass normal divergence and/or fluctuations as would be appreciated by one of ordinary skill in the relevant art. In certain embodiments, the term “approximately” or “about” can refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, 1%, 0.5%, 0.1, or less in either direction (greater than or less than) of a stated reference value unless otherwise stated or otherwise evident from the context (except where such number would exceed 100% of a possible value).

As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, or subcutaneous route). Administration of a nanoparticle composition to a mammal or mammalian cell may involve contacting one or more cells with the nanoparticle composition.

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of therapeutic and/or prophylactic are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, the terms “editing efficiency”, “editing percentage”, “indel efficiency”, and “percent indels” refer to the total number of sequence reads with insertions or deletions relative to the total number of sequence reads. For example, editing efficiency at a target location in a genome may be measured by isolating and sequencing genomic DNA to identify the presence of insertions and deletions introduced by gene editing. In some embodiments, editing efficiency is measured as a percentage of cells that no longer contain a gene (e.g., CD3) after treatment, relative to the number of the cells that initially contained that gene (e.g., CD3+ cells).

As used herein, “knockdown” refers to a decrease in expression of a particular gene product (e.g., protein, mRNA, or both). Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include sequencing of mRNA isolated from a sample of interest. In some embodiments, “knockdown” may refer to some loss of expression of a particular gene product, for example a decrease in the amount of mRNA transcribed or a decrease in the amount of protein expressed by a population of cells (including in vivo populations such as those found in tissues).

As used herein, “knockout” refers to a loss of expression from a particular gene or of a particular protein in a cell. Knockout can be measured by detecting total cellular amount of a protein in a cell, a tissue or a population of cells, for example. Knockout can also be detected at the genome or mRNA level, for example.

As used herein, the term “biodegradable” is used to refer to materials that, when introduced into cells, are broken down by cellular machinery (e.g., enzymatic degradation) or by hydrolysis into components that cells can either reuse or dispose of without significant toxic effect(s) on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis.

As used herein, the “N/P ratio” is the molar ratio of ionizable nitrogen atom-containing lipid (e.g. Compound of Formula I) to phosphate groups in RNA, e.g., in a nanoparticle composition including a lipid component and an RNA.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, the “polydispersity index” is a ratio that describes the homogeneity of the particle size distribution of a system. A small value, e.g., less than 0.3, indicates a narrow particle size distribution. In some embodiments, the polydispersity index may be less than 0.1.

As used herein, “transfection” refers to the introduction of a species (e.g., an RNA) into a cell. Transfection may occur, for example, in vitro, ex vivo, or in vivo.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched (i.e., linear). The alkyl group can also be substituted or unsubstituted. For example, the alkyl group can be substituted with one or more groups including, but not limited to, alkyl, aryl, heteroaryl, cycloalkyl, alkoxy, amino, ether, halide, hydroxy, nitro, silyl, sulfoxo, sulfonate, carboxylate, or thiol, as described herein. A “lower alkyl” group is an alkyl group containing from one to six (e.g., from one to four) carbon atoms.

The term “alkenyl”, as used herein, refers to an aliphatic group containing at least one carbon-carbon double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, an alkenyl group may be substituted by one or more alkyl, carbocyclyl, aryl, heterocyclyl, or heteroaryl groups is contemplated. Exemplary alkenyl groups include, but are not limited to, vinyl (—CH═CH₂), allyl (—CH₂CH═CH₂), cyclopentenyl (—C₅H7), and 5-hexenyl (—CH₂CH₂CH₂CH₂CH═CH₂).

An “alkylene” group refers to a divalent alkyl radical, which may be branched or unbranched (i.e., linear). Any of the above mentioned monovalent alkyl groups may be converted to an alkylene by abstraction of a second hydrogen atom from the alkyl. Representative alkylenes include C₂₋₄ alkylene and C₂₋₃ alkylene. Typical alkylene groups include, but are not limited to —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like. The alkylene group can also be substituted or unsubstituted. For example, the alkylene group can be substituted with one or more groups including, but not limited to, alkyl, aryl, heteroaryl, cycloalkyl, alkoxy, amino, ether, halide, hydroxy, nitro, silyl, sulfoxo, sulfonate, carboxylate, or thiol, as described herein.

The term “alkenylene” includes divalent, straight or branched, unsaturated, acyclic hydrocarbyl groups having at least one carbon-carbon double bond and, in one embodiment, no carbon-carbon triple bonds. Any of the above-mentioned monovalent alkenyl gorups may be converted to an alkenylene by abstraction of a second hydrogen atom from the alkenyl. Representative alkenylenes include C₂₋₆alkenylenes.

The term “C_{x-y}” when used in conjunction with a chemical moiety, such as alkyl or alkylene, is meant to include groups that contain from x to y carbons in the chain. For example, the term “C_{x-y} alkyl” refers to substituted or unsubstituted saturated hydrocarbon groups, including straight-chain and branched-chain alkyl and alkylene groups that contain from x to y carbons in the chain.

The term “alkoxy” refers to an alkyl group, preferably a lower alkyl group, having an oxygen attached thereto. Representative alkoxy groups include methoxy, ethoxy, propoxy, tert-butoxy and the like.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicant reserves the right to physically incorporate into this application any and all materials and information from any such articles, patents, patent applications, or other physical and electronic documents.

### EXAMPLES

### Example 1—Materials and Methods

### Example 1.1 Lipid Nanoparticle (“LNP”) Formulation

The LNP components were dissolved in 100% ethanol at various molar ratios. The RNA cargos (e.g., Cas9 mRNA and sgRNA combined) were dissolved in 25 mM citrate, 100 mM NaCl, pH 5.0, resulting in a concentration of RNA cargo of approximately 0.45 mg/mL. The LNPs were formulated with a lipid amine to RNA phosphate (N:P) molar ratio of about 6, and a ratio of sgRNA to Cas9 mRNA at 1:2 ratio by weight unless otherwise specified.

LNPs were prepared using various amine lipids in a 4-component lipid system. Unless otherwise specified, the LNPs contained ionizable lipid, Compound 3, nonyl 8-((7,7-bis(octyloxy)heptyl)(2-hydroxyethyl)amino)octanoate, DSPC, cholesterol, and PEG2k-DMG.

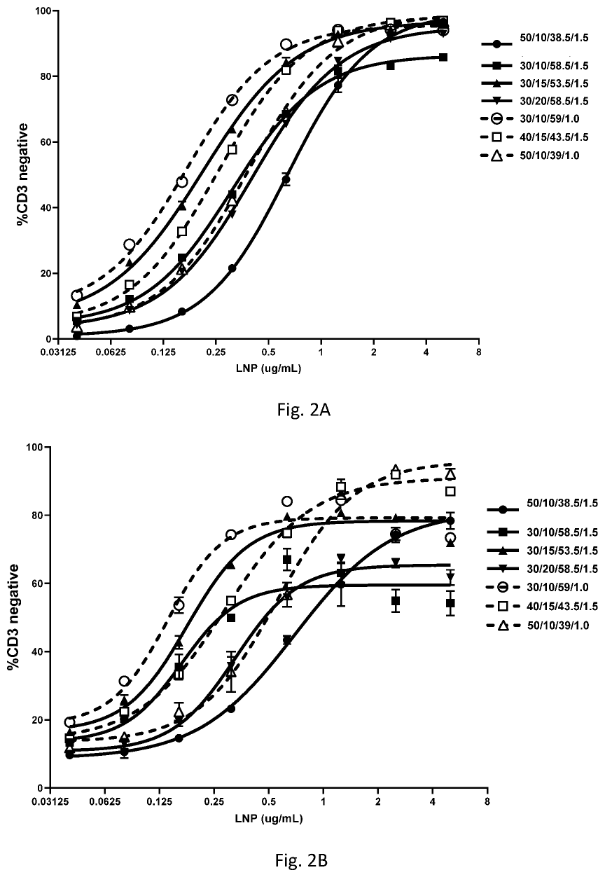

LNPs were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2). The LNPs were held for 1 hour at room temperature, and further diluted with water (approximately 1:1 v/v). LNPs were concentrated using tangential flow filtration, e.g., on a flat sheet cartridge (Sartorius, 100 kD MWCO) and optionally buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

### Example 1.2 In Vitro Transcription (“IVT”) of Nuclease mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation region was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID Nos: 1-3 (see sequences in Table 17). When the sequences cited in this paragraph are referred to below with respect to RNAs, it is understood that Ts should be replaced with Us (which can be modified nucleosides as described above). Messenger RNAs used in the Examples include a 5′ cap and a 3′ polyadenylation sequence e.g., up to 100 nts and are identified in Table 17. Guide RNAs are chemically synthesized by methods known in the art.

### Example 1.3 Formulation Analytics

Dynamic Light Scattering (“DLS”) was used to characterize the polydispersity index (“pdi”) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

Asymmetric-Flow Field Flow Fractionation—Multi-Angle Light Scattering (AF4-MALS) is used to separate particles in the composition by hydrodynamic radius and then measure the molecular weights, hydrodynamic radii and root mean square radii of the fractionated particles. This allows the ability to assess molecular weight and size distributions as well as secondary characteristics such as the Burchard-Stockmeyer Plot (ratio of root mean square (“rms”) radius to hydrodynamic radius over time suggesting the internal core density of a particle) and the rms conformation plot (log of rms radius vs log of molecular weight where the slope of the resulting linear fit gives a degree of compactness vs elongation). Cryo-electron microscopy (“cryo-EM”) can be used to determine the particle size, morphology, and structural characteristics of an LNP.

Lipid compositional analysis of the LNPs was determined from liquid chromatography followed by charged aerosol detection (LC-CAD). This analysis can provide a comparison of the actual lipid content versus the nominal lipid content.

LNP compositions were analyzed for average particle size, polydispersity index (pdi), total RNA content, encapsulation efficiency of RNA, and zeta potential. LNP compositions may be further characterized by lipid analysis, AF4-MALS, NTA, and/or cryo-EM. Average particle size and polydispersity were measured by dynamic light scattering (DLS) using a Malvern Zetasizer DLS instrument. LNP samples were diluted with PBS buffer prior to being measured by DLS. Z-average diameter was reported along with number average diameter and pdi. The Z average is the intensity weighted mean hydrodynamic size of the ensemble collection of particles and was measured by dynamic light scattering. The number average is the particle number weighted mean hydrodynamic size of the ensemble collection of particles measured by dynamic light scattering. A Malvern Zetasizer instrument was also used to measure the zeta potential of the LNP. Samples were diluted 1:17 (50 μL into 800 μL) in 0.1×PBS, pH 7.4 prior to measurement.

Encapsulation efficiency was calculated as (Total RNA−Free RNA)/Total RNA.

A fluorescence-based assay (Ribogreen®, ThermoFisher Scientific) was used to determine total RNA concentration and free RNA. LNP samples were diluted appropriately with 1× TE buffer containing 0.2% Triton-X¹⁰⁰ to determine total RNA or 1× TE buffer to determine free RNA. Standard curves were prepared by utilizing the starting RNA solution used to make the compositions and diluted in 1× TE buffer+/−0.2% Triton-X 100. Diluted RiboGreen® dye (according to the manufacturer's instructions) was then added to each of the standards and samples and allowed to incubate for approximately 10 minutes at room temperature, in the absence of light. A SpectraMax M5 Microplate Reader (Molecular Devices) was used to read the samples with excitation, auto cutoff and emission wavelengths set to 488 nm, 515 nm, and 525 nm respectively. Total RNA and free RNA were determined from the appropriate standard curves.

AF4-MALS is used to look at molecular weight and size distributions as well as secondary statistics from those calculations. LNPs are diluted as appropriate and injected into a AF4 separation channel using an HPLC autosampler where they are focused and then eluted with an exponential gradient in cross flow across the channel. All fluid is driven by an HPLC pump and Wyatt Eclipse Instrument. Particles eluting from the AF4 channel flow through a UV detector, multi-angle light scattering detector, quasi-elastic light scattering detector and differential refractive index detector. Raw data is processed by using a Debeye model to determine molecular weight and rms radius from the detector signals.

CAD is a destructive mass-based detector which detects all non-volatile compounds and the signal is consistent regardless of analyte structure.

Lipid components in LNPs were analyzed quantitatively by HPLC coupled to a charged aerosol detector (CAD). Chromatographic separation of 4 lipid components was achieved by reverse phase HPLC.

### Example 1.4 T Cell Preparation

Healthy human donor apheresis was obtained commercially (Hemacare). T cells were isolated by negative selection using the EasySep Human T cell Isolation Kit (Stem Cell Technology, Cat. 17951) or by CD4/CD8 positive selection using the StraightFrom® Leukopak® CD4/CD8 MicroBeads (Milteny, Catalog #130-122-352) on the MultiMACS™ Cell24 Separator Plus instrument following manufacturers instruction. T cells were cryopreserved in Cryostor CS10 freezing media (Cat. #07930) for future use. Upon thaw, T cells were cultured in complete T cell growth media composed of CTS OpTmizer Base Media (CTS OpTmizer Media (Gibco, A3705001) supplemented with 1× GlutaMAX, 10 mM HEPES buffer (10 mM), and 1% pen-strep (Gibco, 15140-122) further supplemented with 200 IU/mL IL2 (Peprotech, 200-02), 5 ng/mL IL7 (Peprotech, 200-07), ng/mL IL15 (Peprotech, 200-15), and 2.5% human serum (Gemini, 100-512). After overnight rest, T cells at a density of 10⁶/mL were activated with T cell TransAct Reagent (1:100 dilution, Miltenyi) and incubated at 37° C. for 24 or 48 hours. Post incubation, cells at a density of 0.5×10⁶/mL were used for editing applications.

Unless otherwise indicated, the same process was used for non-activated T cells with the following exceptions. Upon thaw, non-Activated T cells were cultured in the CTS complete growth media composed of CTS OpTmizer Base Media (Thermofisher, A10485-01), 1% pen-strep (Corning, 30-002-CI) 1× GlutaMAX (Thermofisher, 35050061), 10 mM HEPES (Thermofisher, 15630080)) which was further supplemented with 200 U/mL IL2 (Peprotech, 200-02), 5 ng/mL IL7 (Peprotech, 200-07), 5 ng/mL IL15 (Peprotech, 200-15) with 5% human AB serum (Gemini, 100-512) were incubated for 24 hrs with no activation. T cells were plated at a cell density of 10⁶/mL in 100 uL of CTS OpTmizer base media, described above, containing 2.5% human serum and cytokines for editing applications.

### Example 1.5 LNP Transfection of T Cells

T cells were transfected with LNPs formulated as described in Example 1.1. Materials used for LNP transfection are noted in Table 1. The LNP dose response curves (DRCs) transfection was performed on the Hamilton Microlab STAR liquid handling system. The liquid handler was provided with the following: (a) 4× the desired highest LNP dose in the top row of a deep well 96-deep well plate, (b) ApoE3 diluted in media at 20 μg/mL, (c) complete T cell growth media composed of CTS OpTmizer Base Media as previously described in Example 1 and (d) T cells plated at 10⁶/ml density in 100 μL in 96-well flat bottom tissue culture plates. The liquid handler first performed an 8-point two-fold serial dilution of the LNPs starting from the 4× LNP dose in the deep well plate. Equal volume of ApoE3 media was then added to each well resulting in a 1:1 dilution of both LNP and ApoE3. Subsequently, 100 μL of the LNP-ApoE mix was added to each T cell plate. The final concentration of LNPs at the top dose was set to be 5 μg/mL. Final concentrations of ApoE3 at 5 μg/mL and T cells were at a final density of 0.5×10⁶ cells/mL. Plates were incubated at 37° C. with 5% CO₂ for 24 or 48 hours for activated or on-activated T cells, respectively. Post-incubation, T cells treated with LNPs were harvested and analyzed for on-target editing or Cas9 protein expression detection. Remaining cells were cultured for 7-10 days post LNP treatment and protein surface expression assessed by flow cytometry.

### Example 1.7 Flow Cytometry Analysis

The T cell receptor alpha chain encoded by TRAC is required for T cell receptor/CD3 complex assembly and translocation to the cell surface. Editing was assayed by an increase in the percentage of CD3 negative cells following editing. To assay cell surface proteins by flow cytometry, T cells were resuspended in 100 μL of an antibody cocktail (1:100 PE-anti-human CD3 [Biolegend, Cat.300441], 1:200 FITC anti-human CD4[Biolegend, Cat.300538], 1:200 APC anti-human CD8a [Biolegend, Cat.301049], FACS buffer [PBS+2% FBS+2 mM EDTA]) and incubated for 30 mins at 4° C. T cells were washed then resuspended in FACS buffer (PBS+2% FBS+2 mM EDTA). T cells were subsequently processed on a Cytoflex instrument (Beckman Coulter). Data analysis was performed using FlowJo software package (v.10.6.1 or v.10.7.1). Briefly, T cells were gated on lymphocytes followed by single cells. These single cells were gated on CD4+/CD8+ status from which CD8+/CD3− cells were selected. Percent of CD8+/CD3− cells were quantified to determine the percentage of the cell population in which the edited target locus resulted in TCR knockout. A linear regression model was used to generate dose response curves for TCR KO using Prism GraphPad (v.9.0). The half maximal effective concentration (EC₅₀) and maximum percent CD3− value of the curve were calculated for each LNP.

### Example 1.6. Next-Generation Sequencing (“NGS”) and Analysis for Editing Efficiency

To quantitatively determine the efficiency of editing at the target location in the genome, sequencing was utilized to identify the presence of insertions and deletions introduced by gene editing. PCR primers were designed around the target site within the gene of interest (e.g. AAVS1), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

### Example 2—Compound 3 Composition Screens in T Cells

2.1 Characterization of LNP Ionizable Lipid in CD3+ T Cells

To evaluate editing efficacy, T cells were treated with LNP compositions with varied mol percents of lipid components encapsulating Cas9 mRNA and a sgRNA targeting the TRAC gene and assessed by flow cytometry for loss of T cell receptor surface proteins.

LNPs were generally prepared as described in Example 1 with the lipid composition expressed as the molar ratio of Compound 3/DSPC/cholesterol/PEG, respectively, as indicated in Table 1. LNP delivered mRNA encoding Cas9 (SEQ ID No. 4) and sgRNA (SEQ ID NO. 10) targeting human TRAC. The cargo ratio of sgRNA to Cas9 mRNA was 1:2 by weight.

LNP formulations were analyzed for Z-average and number average particle size, polydispersity (pdi), total RNA content and encapsulation efficiency of RNA as described in Example 1 and results shown in Table 1.

TABLE 1
LNP formulation analysis results

| | | | | | Num | |
|---|---|---|---|---|---|---|
| | | Encap- | Z-Ave | | Ave | |
| | Nominal | sulation | Size | | Size | N/P |
| LNP ID | Molar Ratio | (%) | (nm) | PDI | (nm) | Ratio |
| COMPOSITION | 50/10/38.5/1.5 | 94% | 73 | 0.07 | 56 | 6 |
| 1 | | | | | | |
| COMPOSITION | 50/5/43.5/1.5 | 94% | 71 | 0.05 | 54 | 6 |
| 2 | | | | | | |
| COMPOSITION | 45/15/38.5/1.5 | 95% | 80 | 0.05 | 62 | 6 |
| 3 | | | | | | |
| COMPOSITION | 45/5/48.5/1.5 | 95% | 67 | 0.08 | 49 | 6 |
| 4 | | | | | | |
| COMPOSITION | 40/10/48.5/1.5 | 94% | 68 | 0.1 | 46 | 6 |
| 5 | | | | | | |
| COMPOSITION | 30/10/58.5/1.5 | 98% | 71 | 0.06 | 56 | 6 |
| 6 | | | | | | |
| COMPOSITION | 30/5/63.5/1.5 | 98% | 65 | 0.04 | 50 | 6 |
| 7 | | | | | | |
| COMPOSITION | 55/5/38.5/1.5 | 92% | 92 | 0 | 76 | 6 |
| 8 | | | | | | |
| COMPOSITION | 55/10/33.5/1.5 | 92% | 84 | 0.05 | 65 | 6 |
| 9 | | | | | | |
| COMPOSITION | 65/5/28.5/1.5 | 83% | 122* | 0.02 | 102 | 6 |
| 10 | | | | | | |
| COMPOSITION | 50/10/38.5/1.5 | 94% | 72 | 0.06 | 56 | 5 |
| 11 | | | | | | |
| COMPOSITION | 50/10/38.5/1.5 | 96% | 71 | 0.04 | 57 | 7 |
| 11 | | | | | | |

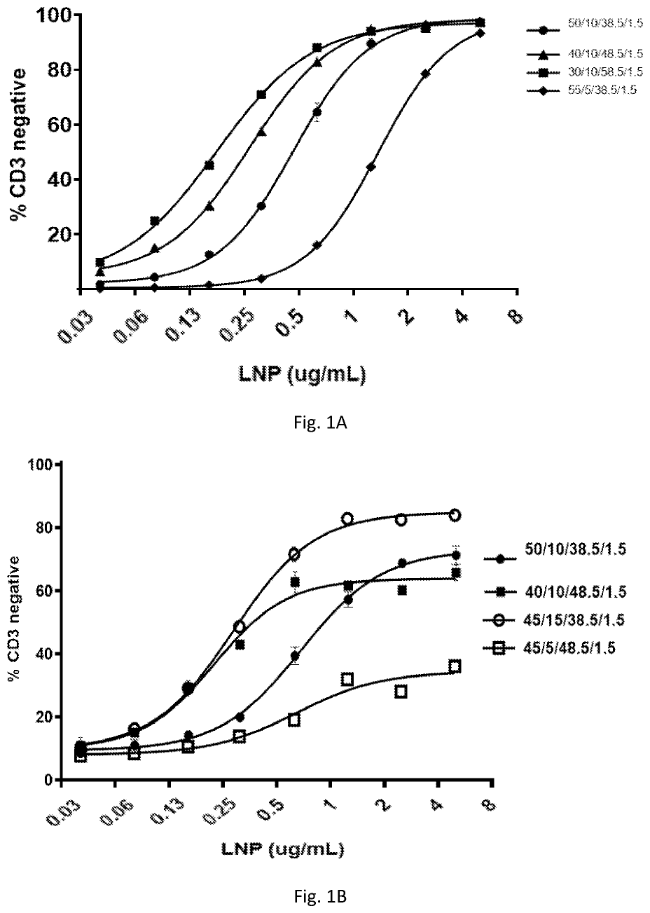

LNPs in Table 1 were assessed to determine the effect of the LNP composition ratios on editing efficiency in CD3 positive T cells. T cells from two donors (Lot #W106 and #W0186) were prepared and transfected as described in Example 1 for activated T cells and non-activated T cells, respectively. Seven days post transfection, the edited T cells were harvested and phenotyped by flow cytometry as described in Example 1. The percentage of CD3 negative T cells were measured following treatment with LNP concentrations of 0.04 μg/mL, 0.08 μg/mL, 0.16 μg/mL, 0.25 μg/mL, 0.63 μg/mL, 1.25 μg/mL, 2.5 μg/mL, and 5 μg/mL. The mean percent CD3 negative T cells, maximum percent CD3 negative value, and EC50 at each LNP dose is shown in Table 2 and FIG. 1A for activated T cells and Table 3 and FIG. 1B for non-activated T cells. Approximate max % CD3− or EC50 values are noted with a tilde and values that could not be determined with an “ND”.

TABLE 2
Mean percent CD3 negative cells following treatment of
activated T cell with indicated LNP formulations.

| | | LNP | Mean | | | Max | |
|---|---|---|---|---|---|---|---|
| | | (μg/ | % | | | % | |
| LNP ID | LNP | mL) | CD3- | SD | N | CD3- | EC50 |
| COMPOSITION | 50/10/38.5/1.5 | 5 | 97.6 | 0.0 | 2 | 99.0 | 0.47 |
| 1 | | 2.5 | 96.2 | 1.1 | 2 | | |
| | | 1.25 | 89.6 | 1.9 | 2 | | |
| | | 0.63 | 64.6 | 3.4 | 2 | | |
| | | 0.31 | 30.4 | 1.0 | 2 | | |
| | | 0.16 | 12.7 | 1.0 | 2 | | |
| | | 0.08 | 4.4 | 0.7 | 2 | | |
| | | 0.04 | 1.8 | 0.3 | 2 | | |
| COMPOSITION | 50/5/43.5/1.5 | 5 | 91.3 | 0.3 | 2 | 95.9 | 1.22 |
| 2 | | 2.5 | 81.7 | 0.9 | 2 | | |
| | | 1.25 | 48.3 | 1.1 | 2 | | |
| | | 0.63 | 18.9 | 2.4 | 2 | | |
| | | 0.31 | 5.5 | 0.6 | 2 | | |
| | | 0.16 | 2.0 | 0.3 | 2 | | |
| | | 0.08 | 0.8 | 0.2 | 2 | | |
| | | 0.04 | 0.4 | 0.1 | 2 | | |
| COMPOSITION | 45/15/38.5/1.5 | 5 | 97.9 | 0.1 | 2 | 96.8 | 0.24 |
| 3 | | 2.5 | 97.3 | 0.4 | 2 | | |
| | | 1.25 | 96.3 | 0.3 | 2 | | |
| | | 0.63 | 87.8 | 0.6 | 2 | | |
| | | 0.31 | 62.3 | 2.5 | 2 | | |
| | | 0.16 | 35.0 | 0.7 | 2 | | |
| | | 0.08 | 15.1 | 1.7 | 2 | | |
| | | 0.04 | 6.7 | 0.2 | 2 | | |
| COMPOSITION | 45/5/48.5/1.5 | 5 | 91.8 | 0.2 | 2 | 96.3 | 0.99 |
| 4 | | 2.5 | 82.8 | 0.2 | 2 | | |
| | | 1.25 | 58.8 | 0.9 | 2 | | |
| | | 0.63 | 28.9 | 1.3 | 2 | | |
| | | 0.31 | 11.0 | 1.5 | 2 | | |
| | | 0.16 | 4.2 | 0.0 | 2 | | |
| | | 0.08 | 1.7 | 0.5 | 2 | | |
| | | 0.04 | 1.1 | 0.4 | 2 | | |
| COMPOSITION | 40/10/48.5/1.5 | 5 | 97.6 | 0.2 | 2 | 98.5 | 0.27 |
| 5 | | 2.5 | 96.6 | 0.8 | 2 | | |
| | | 1.25 | 94.8 | 0.2 | 2 | | |
| | | 0.63 | 83.0 | 0.1 | 2 | | |
| | | 0.31 | 57.7 | 0.4 | 2 | | |
| | | 0.16 | 30.6 | 0.4 | 2 | | |
| | | 0.08 | 15.2 | 1.0 | 2 | | |
| | | 0.04 | 6.6 | 0.1 | 2 | | |
| COMPOSITION | 30/10/58.5/1.5 | 5 | 97.2 | 0.4 | 2 | 97.3 | 0.18 |
| 6 | | 2.5 | 95.2 | 0.7 | 2 | | |
| | | 1.25 | 94.0 | 1.2 | 2 | | |
| | | 0.63 | 88.1 | 0.4 | 2 | | |
| | | 0.31 | 71.0 | 1.1 | 2 | | |
| | | 0.16 | 45.2 | 0.7 | 2 | | |
| | | 0.08 | 25.0 | 0.7 | 2 | | |
| | | 0.04 | 9.9 | 0.0 | 2 | | |
| COMPOSITION | 30/5/63.5/1.5 | 5 | 82.9 | 0.3 | 2 | 88.1 | 0.70 |
| 7 | | 2.5 | 71.5 | 0.2 | 2 | | |
| | | 1.25 | 61.2 | 1.4 | 2 | | |
| | | 0.63 | 39.9 | 0.7 | 2 | | |
| | | 0.31 | 22.5 | 0.4 | 2 | | |
| | | 0.16 | 9.6 | 0.9 | 2 | | |
| | | 0.08 | 3.5 | 0.5 | 2 | | |
| | | 0.04 | 2.1 | 0.5 | 2 | | |
| COMPOSITION | 55/5/38.5/1.5 | 5 | 93.4 | 0.7 | 2 | 99.0 | 1.37 |
| 8 | | 2.5 | 78.6 | 1.5 | 2 | | |
| | | 1.25 | 44.6 | 0.1 | 2 | | |
| | | 0.63 | 16.2 | 1.1 | 2 | | |
| | | 0.31 | 3.9 | 0.2 | 2 | | |
| | | 0.16 | 1.6 | 0.1 | 2 | | |
| | | 0.08 | 0.7 | 0.1 | 2 | | |
| | | 0.04 | 0.4 | 0.0 | 2 | | |
| COMPOSITION | 55/10/33.5/1.5 | 5 | 97.1 | 0.4 | 2 | 98.9 | 0.54 |
| 9 | | 2.5 | 96.9 | 0.1 | 2 | | |
| | | 1.25 | 89.0 | 0.2 | 2 | | |
| | | 0.63 | 58.1 | 3.6 | 2 | | |
| | | 0.31 | 22.7 | 2.9 | 2 | | |
| | | 0.16 | 9.1 | 1.6 | 2 | | |
| | | 0.08 | 3.3 | 1.1 | 2 | | |
| | | 0.04 | 1.1 | 0.0 | 2 | | |
| COMPOSITION | 65/5/28.5/1.5 | 5 | 42.7 | 0.3 | 2 | 84.5 | 4.98 |
| 10 | | 2.5 | 10.9 | 0.4 | 2 | | |
| | | 1.25 | 2.0 | 0.4 | 2 | | |
| | | 0.63 | 0.5 | 0.0 | 2 | | |
| | | 0.31 | 0.3 | 0.1 | 2 | | |
| | | 0.16 | 0.4 | 0.1 | 2 | | |
| | | 0.08 | 0.2 | 0.0 | 2 | | |
| | | 0.04 | 0.4 | 0.1 | 2 | | |
| COMPOSITION | 50/10/38.5/1.5 | 5 | 97.0 | 0.3 | 2 | 100.0 | 0.57 |
| 11 | (N/P: 5.0) | 2.5 | 96.3 | 0.2 | 2 | | |
| | | 1.25 | 82.6 | 2.7 | 2 | | |
| | | 0.63 | 54.3 | 1.3 | 2 | | |
| | | 0.31 | 25.2 | 2.1 | 2 | | |
| | | 0.16 | 10.0 | 0.9 | 2 | | |
| | | 0.08 | 2.7 | 0.7 | 2 | | |
| | | 0.04 | 1.4 | 0.4 | 2 | | |
| COMPOSITION | 50/10/38.5/1.5 | 5 | 97.9 | 0.7 | 2 | 99.3 | 0.35 |
| 11 | (N/P: 7.0) | 2.5 | 97.3 | 0.1 | 2 | | |
| | | 1.25 | 94.3 | 0.6 | 2 | | |
| | | 0.63 | 77.7 | 0.9 | 2 | | |
| | | 0.31 | 43.5 | 2.5 | 2 | | |
| | | 0.16 | 21.2 | 1.3 | 2 | | |
| | | 0.08 | 8.4 | 1.1 | 2 | | |
| | | 0.04 | 2.7 | 0.3 | 2 | | |

TABLE 3
Mean percent CD3 negative cells following treatment of
non-activated T cell with indicated LNP formulations.

| | | LNP | Mean | | | Max | |
|---|---|---|---|---|---|---|---|
| | | (μg/ | % | | | % | |
| LNP ID | LNP | mL) | CD3- | SD | N | CD3- | EC50 |
| COMPOSITION | 50/5/43.5/1.5 | 5 | 70.7 | 2.9 | 2 | 99.0 | 0.68 |
| 1 | | 2.5 | 68.2 | 1.2 | 2 | | |
| | | 1.25 | 56.8 | 2.4 | 2 | | |
| | | 0.63 | 39.1 | 2.8 | 2 | | |
| | | 0.31 | 19.8 | 0.3 | 2 | | |
| | | 0.16 | 14.1 | 0.3 | 2 | | |
| | | 0.08 | 11.0 | 1.8 | 2 | | |
| | | 0.04 | 8.5 | 0.4 | 2 | | |
| COMPOSITION | 50/5/43.5/1.5 | 5 | 38.5 | 4.9 | 2 | 95.9 | 1.45 |
| 2 | | 2.5 | 27.1 | 0.1 | 2 | | |
| | | 1.25 | 28.0 | 0.1 | 2 | | |
| | | 0.63 | 13.6 | 1.3 | 2 | | |
| | | 0.31 | 10.6 | 1.8 | 2 | | |
| | | 0.16 | 10.7 | 2.3 | 2 | | |
| | | 0.08 | 9.1 | 1.8 | 2 | | |
| | | 0.04 | 10.1 | 3.1 | 2 | | |
| COMPOSITION | 45/15/ | 5 | 84.0 | 0.5 | 2 | 98.8 | 0.29 |
| 3 | 38.5/1.5 | 2.5 | 82.6 | 1.2 | 2 | | |
| | | 1.25 | 82.9 | 1.8 | 2 | | |
| | | 0.63 | 71.6 | 2.4 | 2 | | |
| | | 0.31 | 48.6 | 0.5 | 2 | | |
| | | 0.16 | 29.1 | 2.4 | 2 | | |
| | | 0.08 | 16.2 | 1.6 | 2 | | |
| | | 0.04 | 10.4 | 3.1 | 2 | | |
| COMPOSITION | 45/5/48.5/1.5 | 5 | 36.1 | 1.1 | 2 | 96.3 | 0.65 |
| 4 | | 2.5 | 28.0 | 0.3 | 2 | | |
| | | 1.25 | 31.9 | 0.4 | 2 | | |
| | | 0.63 | 19.1 | 1.5 | 2 | | |
| | | 0.31 | 13.8 | 0.6 | 2 | | |
| | | 0.16 | 10.6 | 0.3 | 2 | | |
| | | 0.08 | 8.4 | 0.2 | 2 | | |
| | | 0.04 | 7.7 | 1.5 | 2 | | |
| COMPOSITION | 40/10/ | 5 | 65.2 | 2.5 | 2 | 98.5 | 0.22 |
| 5 | 48.5/1.5 | 2.5 | 59.7 | 1.4 | 2 | | |
| | | 1.25 | 61.2 | 1.1 | 2 | | |
| | | 0.63 | 62.3 | 3.3 | 2 | | |
| | | 0.31 | 42.6 | 0.5 | 2 | | |
| | | 0.16 | 29.0 | 0.7 | 2 | | |
| | | 0.08 | 14.9 | 0.5 | 2 | | |
| | | 0.04 | 10.8 | 1.4 | 2 | | |
| COMPOSITION | 30/10/ | 5 | 60.2 | 0.6 | 2 | 97.3 | 0.13 |
| 6 | 58.5/1.5 | 2.5 | 53.3 | 1.1 | 2 | | |
| | | 1.25 | 59.3 | 1.6 | 2 | | |
| | | 0.63 | 65.3 | 0.9 | 2 | | |
| | | 0.31 | 58.9 | 2.0 | 2 | | |
| | | 0.16 | 44.4 | 0.5 | 2 | | |
| | | 0.08 | 22.5 | 0.2 | 2 | | |
| | | 0.04 | 15.7 | 0.5 | 2 | | |
| COMPOSITION | 30/5/63.5/1.5 | 5 | 37.4 | 1.1 | 2 | 88.1 | ~22.14 |
| 7 | | 2.5 | 26.8 | 1.9 | 2 | | |
| | | 1.25 | 23.6 | 2.4 | 2 | | |
| | | 0.63 | 20.0 | 1.4 | 2 | | |
| | | 0.31 | 17.0 | 1.4 | 2 | | |
| | | 0.16 | 11.9 | 2.2 | 2 | | |
| | | 0.08 | 10.1 | 0.4 | 2 | | |
| | | 0.04 | 7.8 | 0.1 | 2 | | |
| COMPOSITION | 55/5/38.5/1.5 | 5 | 55.8 | 2.0 | 2 | 99.0 | 1.35 |
| 8 | | 2.5 | 47.8 | 0.4 | 2 | | |
| | | 1.25 | 32.0 | 2.7 | 2 | | |
| | | 0.63 | 17.4 | 1.9 | 2 | | |
| | | 0.31 | 11.7 | 1.4 | 2 | | |
| | | 0.16 | 9.3 | 1.0 | 2 | | |
| | | 0.08 | 8.0 | 0.5 | 2 | | |
| | | 0.04 | 9.0 | 0.3 | 2 | | |
| COMPOSITION | 55/10/ | 5 | 73.7 | 2.2 | 2 | 98.9 | 0.63 |
| 9 | 33.5/1.5 | 2.5 | 75.2 | 1.4 | 2 | | |
| | | 1.25 | 61.3 | 0.8 | 2 | | |
| | | 0.63 | 42.6 | 2.0 | 2 | | |
| | | 0.31 | 24.6 | 2.3 | 2 | | |
| | | 0.16 | 17.6 | 3.3 | 2 | | |
| | | 0.08 | 10.5 | 1.0 | 2 | | |
| | | 0.04 | 9.0 | 0.9 | 2 | | |
| COMPOSITION | 65/5/28.5/1.5 | 5 | 23.8 | 1.2 | 2 | 84.5 | ~129.4 |
| 10 | | 2.5 | 18.4 | 1.3 | 2 | | |
| | | 1.25 | 16.3 | 1.4 | 2 | | |
| | | 0.63 | 14.7 | 2.3 | 2 | | |
| | | 0.31 | 10.4 | 1.4 | 2 | | |
| | | 0.16 | 10.8 | 0.8 | 2 | | |
| | | 0.08 | 8.4 | 0.2 | 2 | | |
| | | 0.04 | 8.2 | 0.7 | 2 | | |
| COMPOSITION | 50/10/ | 5 | 72.0 | 0.5 | 2 | 100.0 | 1.16 |
| 11 | 38.5/1.5 | 2.5 | 65.3 | 3.3 | 2 | | |
| | (N/P: 5.0) | 1.25 | 46.4 | 0.6 | 2 | | |
| | | 0.63 | 30.3 | 2.0 | 2 | | |
| | | 0.31 | 18.9 | 1.3 | 2 | | |
| | | 0.16 | 12.7 | 0.3 | 2 | | |
| | | 0.08 | 7.1 | 0.1 | 2 | | |
| | | 0.04 | 27.6 | 22.4 | 2 | | |
| COMPOSITION | 50/10/ | 5 | 71.3 | 1.8 | 2 | 99.3 | 0.48 |
| 11 | 38.5/1.5 | 2.5 | 70.6 | 1.7 | 2 | | |
| | (N/P: 7.0) | 1.25 | 63.6 | 0.1 | 2 | | |
| | | 0.63 | 45.3 | 4.9 | 2 | | |
| | | 0.31 | 24.1 | 0.5 | 2 | | |
| | | 0.16 | 18.0 | 0.2 | 2 | | |
| | | 0.08 | 10.1 | 2.4 | 2 | | |
| | | 0.04 | 0.0 | 0.0 | 2 | | |

### Example 3—Selected Compound 3 LNP Compositions Screening in T Cells

3.1 Evaluation of Select LNP Compositions in Edited CD3 Positive T Cells

To evaluate LNP editing efficacy, T cells were treated with LNP compositions with varied molar ratios of lipid components encapsulating Cas9 mRNA and a sgRNA targeting the TRAC gene and assessed by flow cytometry for loss of T cell receptor surface proteins.

LNPs were generally prepared as described in Example 1 with the lipid composition, as indicated in Table 4, expressed as the molar ratio of ionizable lipid A/DSPC/cholesterol/PEG, respectively. LNP delivered mRNA encoding Cas9 (SEQ ID No. 4) and sgRNA (SEQ ID NO. 10) targeting human TRAC. The cargo ratio of sgRNA to Cas9 mRNA was 1:2 by weight.

LNP formulations were analyzed for average particle size, polydispersity (pdi), total RNA content and encapsulation efficiency of RNA as described in Example 1 and results shown in Table 4.

TABLE 4
LNP formulation analysis results

| | | | | | Num | |
|---|---|---|---|---|---|---|
| | Nominal | Encap- | Z-Ave | | Ave | |
| | Molar | sulation | Size | | Size | N/P |
| LNP ID | Ratio | (%) | (nm) | PDI | (nm) | Ratio |
| COMPOSITION | 50/10/38.5/1.5 | 97% | 68 | 0.04 | 59 | 6 |
| 1 | | | | | | |
| (Comparative) | | | | | | |
| COMPOSITION | 30/10/58.5/1.5 | 98% | 76 | 0.06 | 65 | 6 |
| 6 | | | | | | |
| COMPOSITION | 30/15/53.5/1.5 | 99% | 83 | 0.03 | 73 | 6 |
| 12 | | | | | | |
| COMPOSITION | 30/20/58.5/1.5 | 98% | 86 | 0.07 | 77 | 6 |
| 13 | | | | | | |
| COMPOSITION | 30/10/59/1.0 | 98% | 88 | 0.04 | 75 | 6 |
| 14 | | | | | | |
| COMPOSITION | 45/10/43.5/1.5 | 98% | 65 | 0.06 | 56 | 6 |
| 15 | | | | | | |
| COMPOSITION | 40/15/43.5/1.5 | 98% | 74 | 0.03 | 65 | 6 |
| 16 | | | | | | |
| COMPOSITION | 50/10/39/1.0 | 98% | 71 | 0.04 | 64 | 6 |
| 17 | | | | | | |
| COMPOSITION | 30/10/58.5/1.5 | 98% | 73 | 0.08 | 57 | 9 |
| 18 | | | | | | |

T cells from two donors (Lot #W106 and #W790) were prepared and transfected as described in Example 1 for activated T cells and non-activated T cells, respectively. Seven days post transfection, the edited T cells were harvested and phenotyped by flow cytometry as described in Example 1.

The percentage of CD3 negative T cells was measured following treatment with LNP concentrations of 0.04 μg/mL, 0.08 μg/mL, 0.16 μg/mL, 0.25 μg/mL, 0.63 μg/mL, 1.25 μg/mL, 2.5 μg/mL, and 5 μg/mL. The mean percent CD3 negative T cells, calculated at each LNP dose with corresponding EC50 and maximum value is shown in Table 5 and FIG. 2A for activated T cells and Table 6 and FIG. 2B for non-activated T cells.

TABLE 5
Percent CD3 negative cells following treatment of activated
T cells by LNPs with indicated LNP formulations

| | | LNP | Mean | | | Max | |
|---|---|---|---|---|---|---|---|
| | | (μg/ | % | | | % | |
| LNP ID | LNP | mL) | CD3- | SD | N | CD3- | EC50 |
| COMPOSITION | 50/10/38.5/1.5 | 5 | 96.6 | 0.4 | 2 | 99.5 | 0.64 |
| 1 | | 2.5 | 93.0 | 0.1 | 2 | | |
| (Comparative) | | 1.25 | 77.3 | 2.2 | 2 | | |
| | | 0.63 | 48.6 | 1.9 | 2 | | |
| | | 0.31 | 21.6 | 1.1 | 2 | | |
| | | 0.16 | 8.3 | 0.8 | 2 | | |
| | | 0.08 | 3.1 | 0.0 | 2 | | |
| | | 0.04 | 0.9 | 0.1 | 2 | | |
| COMPOSITION | 30/10/58.5/1.5 | 5 | 85.8 | 0.4 | 2 | 86.5 | 0.31 |
| 6 | | 2.5 | 83.1 | 1.1 | 2 | | |
| | | 1.25 | 81.6 | 1.8 | 2 | | |
| | | 0.63 | 68.6 | 0.7 | 2 | | |
| | | 0.31 | 44.1 | 0.6 | 2 | | |
| | | 0.16 | 24.8 | 0.4 | 2 | | |
| | | 0.08 | 12.2 | 0.7 | 2 | | |
| | | 0.04 | 5.5 | 0.0 | 2 | | |
| COMPOSITION | 30/15/53.5/1.5 | 5 | 95.7 | 0.0 | 2 | 96.8 | 0.21 |
| 12 | | 2.5 | 94.4 | 0.3 | 2 | | |
| | | 1.25 | 92.9 | 0.7 | 2 | | |
| | | 0.63 | 84.4 | 1.4 | 2 | | |
| | | 0.31 | 63.9 | 0.0 | 2 | | |
| | | 0.16 | 40.6 | 1.2 | 2 | | |
| | | 0.08 | 23.4 | 0.1 | 2 | | |
| | | 0.04 | 10.4 | 0.1 | 2 | | |
| COMPOSITION | 30/20/58.5/1.5 | 5 | 93.0 | 0.8 | 2 | 95.1 | 0.40 |
| 13 | | 2.5 | 91.5 | 0.2 | 2 | | |
| | | 1.25 | 84.7 | 0.2 | 2 | | |
| | | 0.63 | 65.5 | 0.7 | 2 | | |
| | | 0.31 | 37.9 | 0.0 | 2 | | |
| | | 0.16 | 20.5 | 0.2 | 2 | | |
| | | 0.08 | 8.7 | 0.3 | 2 | | |
| | | 0.04 | 4.3 | 0.1 | 2 | | |
| COMPOSITION | 30/10/59/1.0 | 5 | 94.1 | 0.6 | 2 | 95.6 | 0.17 |
| 14 | | 2.5 | 94.3 | 0.7 | 2 | | |
| | | 1.25 | 94.2 | 0.3 | 2 | | |
| | | 0.63 | 89.8 | 0.9 | 2 | | |
| | | 0.31 | 72.8 | 0.8 | 2 | | |
| | | 0.16 | 47.9 | 0.2 | 2 | | |
| | | 0.08 | 28.8 | 0.7 | 2 | | |
| | | 0.04 | 13.2 | 0.3 | 2 | | |
| COMPOSITION | 45/10/43.5/1.5 | 5 | 96.5 | 0.0 | 2 | 98.6 | 0.49 |
| 15 | | 2.5 | 94.5 | 0.5 | 2 | | |
| | | 1.25 | 84.4 | 1.8 | 2 | | |
| | | 0.63 | 60.3 | 1.0 | 2 | | |
| | | 0.31 | 30.6 | 2.4 | 2 | | |
| | | 0.16 | 13.1 | 0.9 | 2 | | |
| | | 0.08 | 5.5 | 0.4 | 2 | | |
| | | 0.04 | 2.0 | 0.4 | 2 | | |
| COMPOSITION | 40/15/43.5/1.5 | 5 | 97.0 | 0.4 | 2 | 98.4 | 0.26 |
| 16 | | 2.5 | 96.4 | 0.3 | 2 | | |
| | | 1.25 | 94.0 | 0.6 | 2 | | |
| | | 0.63 | 82.0 | 1.3 | 2 | | |
| | | 0.31 | 57.7 | 0.4 | 2 | | |
| | | 0.16 | 32.8 | 0.5 | 2 | | |
| | | 0.08 | 16.5 | 1.1 | 2 | | |
| | | 0.04 | 6.9 | 0.2 | 2 | | |
| COMPOSITION | 50/10/39/1.0 | 5 | 96.7 | 0.1 | 2 | 99.2 | 0.38 |
| 17 | | 2.5 | 95.9 | 0.2 | 2 | | |
| | | 1.25 | 90.7 | 1.8 | 2 | | |
| | | 0.63 | 69.3 | 1.4 | 2 | | |
| | | 0.31 | 42.3 | 0.7 | 2 | | |
| | | 0.16 | 21.3 | 0.1 | 2 | | |
| | | 0.08 | 9.8 | 0.3 | 2 | | |
| | | 0.04 | 3.8 | 0.3 | 2 | | |
| COMPOSITION | 30/10/58.5/1.5 | 5 | 46.5 | 3.3 | 2 | 49.7 | 0.72 |
| 18 | (N/P 9.0) | 2.5 | 51.6 | 1.3 | 2 | | |
| | | 1.25 | 42.7 | 0.7 | 2 | | |
| | | 0.63 | 19.7 | 0.6 | 2 | | |
| | | 0.31 | 7.9 | 0.4 | 2 | | |
| | | 0.16 | 3.0 | 0.2 | 2 | | |
| | | 0.08 | 1.0 | 0.1 | 2 | | |
| | | 0.04 | 0.6 | 0.1 | 2 | | |

TABLE 6
Percent CD3 negative cells following treatment of
non-activated T cells with indicated LNP formulations

| | | LNP | Mean | | | Max | |
|---|---|---|---|---|---|---|---|
| | | (μg/ | % | | | % | |
| LNP ID | LNP | mL) | CD3- | SD | N | CD3- | EC50 |
| COMPOSITION | 50/10/38.5/1.5 | 5 | 78.4 | 2.4 | 2 | 81.5 | 0.70 |
| 1 | | 2.5 | 74.3 | 2.2 | 2 | | |
| (Comparative) | | 1.25 | 59.8 | 6.5 | 2 | | |
| | | 0.63 | 43.4 | 1.2 | 2 | | |
| | | 0.31 | 23.3 | 0.1 | 2 | | |
| | | 0.16 | 14.6 | 0.0 | 2 | | |
| | | 0.08 | 10.6 | 1.8 | 2 | | |
| | | 0.04 | 9.7 | 0.8 | 2 | | |
| COMPOSITION | 30/10/58.5/1.5 | 5 | 54.2 | 3.6 | 2 | 59.5 | 0.17 |
| 6 | | 2.5 | 54.9 | 3.3 | 2 | | |
| | | 1.25 | 63.1 | 2.9 | 2 | | |
| | | 0.63 | 67.0 | 3.2 | 2 | | |
| | | 0.31 | 49.9 | 0.1 | 2 | | |
| | | 0.16 | 35.5 | 3.7 | 2 | | |
| | | 0.08 | 20.4 | 1.2 | 2 | | |
| | | 0.04 | 13.4 | 1.1 | 2 | | |
| COMPOSITION | 30/15/53.5/1.5 | 5 | 72.0 | 1.0 | 2 | 78.3 | 0.18 |
| 12 | | 2.5 | 79.4 | 0.5 | 2 | | |
| | | 1.25 | 80.7 | 0.7 | 2 | | |
| | | 0.63 | 79.7 | 1.2 | 2 | | |
| | | 0.31 | 65.5 | 1.0 | 2 | | |
| | | 0.16 | 42.7 | 1.9 | 2 | | |
| | | 0.08 | 25.9 | 1.5 | 2 | | |
| | | 0.04 | 16.5 | 0.9 | 2 | | |
| COMPOSITION | 30/20/58.5/1.5 | 5 | 61.8 | 2.3 | 2 | 65.4 | 0.33 |
| 13 | | 2.5 | 66.0 | 1.3 | 2 | | |
| | | 1.25 | 67.2 | 1.3 | 2 | | |
| | | 0.63 | 55.9 | 0.8 | 2 | | |
| | | 0.31 | 35.7 | 2.8 | 2 | | |
| | | 0.16 | 19.6 | 1.5 | 2 | | |
| | | 0.08 | 13.0 | 1.5 | 2 | | |
| | | 0.04 | 9.9 | 1.1 | 2 | | |
| COMPOSITION | 30/10/59/1.0 | 5 | 73.4 | 0.5 | 2 | 79.3 | 0.14 |
| 14 | | 2.5 | 74.5 | 1.9 | 2 | | |
| | | 1.25 | 84.4 | 0.2 | 2 | | |
| | | 0.63 | 84.1 | 0.9 | 2 | | |
| | | 0.31 | 74.3 | 0.2 | 2 | | |
| | | 0.16 | 53.6 | 2.3 | 2 | | |
| | | 0.08 | 31.4 | 0.9 | 2 | | |
| | | 0.04 | 19.3 | 0.5 | 2 | | |
| COMPOSITION | 45/10/43.5/1.5 | 5 | 75.7 | 0.4 | 2 | 79.1 | 0.63 |
| 15 | | 2.5 | 79.6 | 2.1 | 2 | | |
| | | 1.25 | 66.7 | 1.1 | 2 | | |
| | | 0.63 | 43.9 | 1.6 | 2 | | |
| | | 0.31 | 22.3 | 0.5 | 2 | | |
| | | 0.16 | 14.4 | 0.3 | 2 | | |
| | | 0.08 | 11.5 | 2.6 | 2 | | |
| | | 0.04 | 8.6 | 0.9 | 2 | | |
| COMPOSITION | 40/15/43.5/1.5 | 5 | 87.0 | 0.2 | 2 | 91.0 | 0.29 |
| 16 | | 2.5 | 92.0 | 0.7 | 2 | | |
| | | 1.25 | 88.4 | 2.3 | 2 | | |
| | | 0.63 | 74.8 | 1.3 | 2 | | |
| | | 0.31 | 55.0 | 0.6 | 2 | | |
| | | 0.16 | 33.3 | 1.2 | 2 | | |
| | | 0.08 | 22.4 | 0.2 | 2 | | |
| | | 0.04 | 14.6 | 0.2 | 2 | | |
| COMPOSITION | 50/10/39/1.0 | 5 | 92.2 | 1.5 | 2 | 95.8 | 0.55 |
| 17 | | 2.5 | 93.5 | 0.8 | 2 | | |
| | | 1.25 | 86.1 | 1.2 | 2 | | |
| | | 0.63 | 56.7 | 3.5 | 2 | | |
| | | 0.31 | 34.1 | 5.9 | 2 | | |
| | | 0.16 | 22.4 | 2.6 | 2 | | |
| | | 0.08 | 15.0 | 1.4 | 2 | | |
| | | 0.04 | 11.8 | 1.1 | 2 | | |
| COMPOSITION | 30/10/58.5/1.5 | 5 | 26.1 | 0.4 | 2 | 28.2 | 0.59 |
| 18 | (N/P 9.0) | 2.5 | 28.7 | 0.7 | 2 | | |
| | | 1.25 | 29.3 | 3.3 | 2 | | |
| | | 0.63 | 19.8 | 0.7 | 2 | | |
| | | 0.31 | 17.3 | 2.0 | 2 | | |
| | | 0.16 | 13.3 | 1.2 | 2 | | |
| | | 0.08 | 11.0 | 0.3 | 2 | | |
| | | 0.04 | 14.8 | 7.1 | 2 | | |

### Example 4—Ionizable Lipid Screen in T Cells

4.1 Characterization of LNP Compositions with Various Ionizable Lipids

To evaluate the effect of other ionizable lipids in LNP on editing, T cells were treated with LNP compositions formulated with one of 3 ionizable lipids compounds at two different component ratios. Each of Compound 1, Compound 3, and Compound 4, were formulated in LNPs having a nominal mol % ratio of lipid components: 30% ionizable lipid, 10% DSPC, 59% cholesterol, and 1.0% PEG-2k-DMG, and in comparative LNPs having a nominal mol % ratio of lipid components: 50% ionizable lipid, 10% DSPC, 38.5% cholesterol, and 1.5% PEG-2k-DMG. LNPs encapsulated Cas9 mRNA and a sgRNA targeting the TRAC gene and editing was assessed by flow cytometry for loss of T cell receptor surface proteins.

LNPs were generally prepared as Example 1 with lipid composition ratios expressed as the molar ratio of ionizable lipid/DSPC/cholesterol/PEG, respectively. LNP delivered mRNA encoding Cas9 (SEQ ID No. 5) and sgRNA (SEQ ID NO. 10) targeting human TRAC. The cargo ratio of sgRNA to Cas9 mRNA for the LNPs tested were at 1:1 by weight.

LNP formulations were analyzed for average particle size, polydispersity (pdi), total RNA content and encapsulation efficiency of RNA as described in Example 1 and results shown in Table 7.

TABLE 7
LNP formulation analysis results

| | | Molar | | Z- | | |
|---|---|---|---|---|---|---|
| | | Com- | Encap- | Ave | | |
| | Com- | position | sulation | Size | | N/P |
| LNP ID | pound | ratio | (%) | (nm) | PDI | ratio |
| COMPOSITION 19 | 3 | 50/10/38.5/1.5 | 97% | 72 | 0.05 | 6 |
| (Comparative) | | | | | | |
| COMPOSITION 20 | 1 | 50/10/38.5/1.5 | 97% | 74 | 0.06 | 6 |
| (Comparative) | | | | | | |
| COMPOSITION 21 | 4 | 50/10/38.5/1.5 | 95% | 83 | 0.04 | 6 |
| (Comparative) | | | | | | |
| COMPOSITION 22 | 3 | 30/10/59/1.0 | 99% | 86 | 0.09 | 6 |
| COMPOSITION 23 | 1 | 30/10/59/1.0 | 98% | 84 | 0.04 | 6 |
| COMPOSITION 24 | 4 | 30/10/59/1.0 | 98% | 117 | 0.02 | 6 |

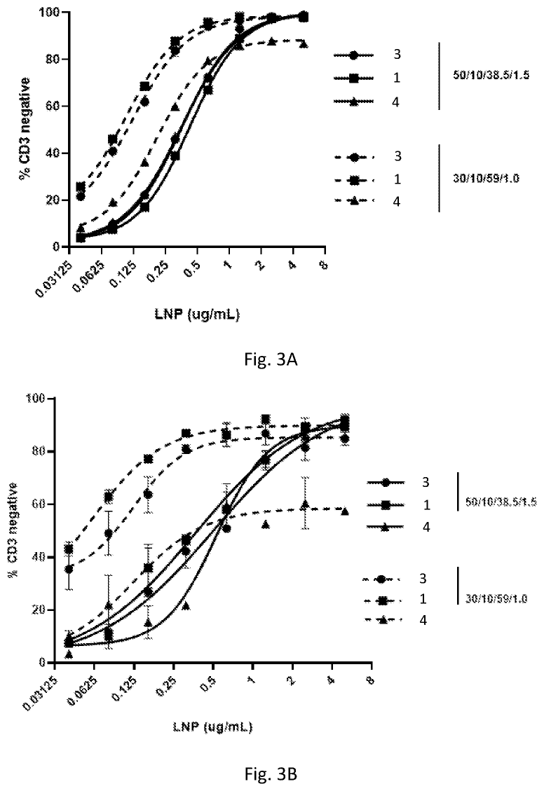

T cells from a single donor (W0106) were generally prepared, activated, and transfected as described in Example 1 except non-activated T cells were rested for 48 hours prior to transfection. Seven days post-transfection, edited activated T cells were harvested and phenotyped by flow cytometry as described in Example 1. The percentage of CD3 negative T cells was measured following treatment with LNP concentrations of 0.04 μg/mL, 0.08 μg/mL, 0.16 μg/mL, 0.25 μg/mL, 0.63 μg/mL, 1.25 μg/mL, 2.5 μg/mL, and 5 μg/mL. The mean percent CD3 negative T cells, maximum percent CD3 value, and EC50 at each LNP dose is shown in Table 8 and FIG. 3A for activated T cells and Table 9 and FIG. 3B for non-activated T cells.

TABLE 8
Percent CD3 negative cells following treatment of activated
T cell with LNPs formulated with different ionizable lipids

| | | Dose | Mean | | | Max | |
|---|---|---|---|---|---|---|---|
| | | (μg/ | % | | | % | |
| LNP | Lipid | mL) | CD3- | SD | N | CD3- | EC50 |
| 50/10/38.5/1.5 | COMPOSITION | 5 | 98.9 | 0 | 2 | 100 | 0.36 |
| (Comparative) | 19 | 2.5 | 98.3 | 0.1 | 2 | | |
| | | 1.25 | 93 | 0.8 | 2 | | |
| | | 0.63 | 72.4 | 0.5 | 2 | | |
| | | 0.31 | 45.9 | 1.3 | 2 | | |
| | | 0.16 | 22.2 | 0.5 | 2 | | |
| | | 0.08 | 9.8 | 0.2 | 2 | | |
| | | 0.04 | 3.8 | 0.4 | 2 | | |
| | COMPOSITION | 5 | 98.2 | 0.2 | 2 | 100 | 0.43 |
| | 20 | 2.5 | 97.7 | 0.1 | 2 | | |
| | | 1.25 | 88.9 | 1.6 | 2 | | |
| | | 0.63 | 67 | 1.1 | 2 | | |
| | | 0.31 | 38.9 | 1.6 | 2 | | |
| | | 0.16 | 17 | 0.1 | 2 | | |
| | | 0.08 | 7.5 | 0.1 | 2 | | |
| | | 0.04 | 4 | 0.6 | 2 | | |
| | COMPOSITION | 5 | 98.5 | 0.3 | 2 | 98.3 | 0.35 |
| | 21 | 2.5 | 97.9 | 0.2 | 2 | | |
| | | 1.25 | 89.7 | 0.8 | 2 | | |
| | | 0.63 | 72.1 | 0 | 2 | | |
| | | 0.31 | 47.1 | 0.3 | 2 | | |
| | | 0.16 | 22.4 | 0.5 | 2 | | |
| | | 0.08 | 10.5 | 0.3 | 2 | | |
| | | 0.04 | 4.6 | 0.1 | 2 | | |
| 30/10/59/1.0 | COMPOSITION | 5 | 97.8 | 0.1 | 2 | 98.3 | 0.12 |
| | 22 | 2.5 | 97.2 | 0.4 | 2 | | |
| | | 1.25 | 96.5 | 0.3 | 2 | | |
| | | 0.63 | 94.1 | 0.1 | 2 | | |
| | | 0.31 | 83.8 | 2.6 | 2 | | |
| | | 0.16 | 61.9 | 1.2 | 2 | | |
| | | 0.08 | 40.9 | 0.5 | 2 | | |
| | | 0.04 | 21.6 | 0.6 | 2 | | |
| | COMPOSITION | 5 | 97.9 | 0.1 | 2 | 98.6 | 0.11 |
| | 23 | 2.5 | 97.5 | 0.2 | 2 | | |
| | | 1.25 | 98.3 | 0.1 | 2 | | |
| | | 0.63 | 95.8 | 0.8 | 2 | | |
| | | 0.31 | 87.8 | 0.8 | 2 | | |
| | | 0.16 | 68.6 | 0.2 | 2 | | |
| | | 0.08 | 46.1 | 1.1 | 2 | | |
| | | 0.04 | 25.7 | 1.7 | 2 | | |
| | COMPOSITION | 5 | 86.8 | 1.6 | 2 | 88.5 | 0.21 |
| | 24 | 2.5 | 88 | 0.4 | 2 | | |
| | | 1.25 | 85.9 | 0.1 | 2 | | |
| | | 0.63 | 79.6 | 0.7 | 2 | | |
| | | 0.31 | 60 | 0.4 | 2 | | |
| | | 0.16 | 36.4 | 0.4 | 2 | | |
| | | 0.08 | 19.2 | 0.2 | 2 | | |
| | | 0.04 | 8.3 | 0.5 | 2 | | |

TABLE 9
Percent CD3 negative cells following treatment of non-activated
T cell with LNPs formulated with different ionizable lipids

| | | Dose | Mean | | | Max | |
|---|---|---|---|---|---|---|---|
| | | (μg/ | % | | | % | |
| LNP | LNP ID | mL) | CD3- | SD | N | CD3- | EC50 |
| 50/10/ | COMPOSITION | 5 | 91.8 | 0.1 | 2 | 100 | 0.5 |
| 38.5/1.5 | 19 | 2.5 | 89.1 | 1.1 | 2 | | |
| (Com- | | 1.25 | 76.8 | 3.7 | 2 | | |
| parative) | | 0.63 | 50.9 | 1.5 | 2 | | |
| | | 0.31 | 42.3 | 6.3 | 2 | | |
| | | 0.16 | 26.9 | 1.7 | 2 | | |
| | | 0.08 | 11.8 | 0.9 | 2 | | |
| | | 0.04 | 7.5 | 0.1 | 2 | | |
| | COMPOSITION | 5 | 91.8 | 1.5 | 2 | 98.6 | 0.37 |
| | 20 | 2.5 | 89.5 | 0.7 | 2 | | |
| | | 1.25 | 76.9 | 2.9 | 2 | | |
| | | 0.63 | 58.1 | 6.6 | 2 | | |
| | | 0.31 | 46.5 | 0.1 | 2 | | |
| | | 0.16 | 36.1 | 8.9 | 2 | | |
| | | 0.08 | 10.6 | 1.8 | 2 | | |
| | | 0.04 | 7.5 | 0.1 | 2 | | |
| | COMPOSITION | 5 | 91.4 | 2.8 | 2 | 89.9 | 0.53 |
| | 21 | 2.5 | 85.8 | 2.2 | 2 | | |
| | | 1.25 | 76.8 | 0.7 | 2 | | |
| | | 0.63 | 59.5 | 8.3 | 2 | | |
| | | 0.31 | 22 | 1 | 2 | | |
| | | 0.16 | 15.5 | 6.2 | 2 | | |
| | | 0.08 | 10.1 | 4.7 | 2 | | |
| | | 0.04 | 3.5 | 0.8 | 2 | | |
| 30/10/ | COMPOSITION | 5 | 84.9 | 2.5 | 2 | 85.4 | 0.12 |
| 59/1.0 | 22 | 2.5 | 81.5 | 4.8 | 2 | | |
| | | 1.25 | 86.8 | 4.3 | 2 | | |
| | | 0.63 | 86.5 | 4.6 | 2 | | |
| | | 0.31 | 81 | 1.7 | 2 | | |
| | | 0.16 | 63.7 | 6.8 | 2 | | |
| | | 0.08 | 49.2 | 8.3 | 2 | | |
| | | 0.04 | 35.5 | 7.7 | 2 | | |
| | COMPOSITION | 5 | 89.3 | 4.2 | 2 | 89.9 | 0.06 |
| | 23 | 2.5 | 89 | 3.8 | 2 | | |
| | | 1.25 | 92.2 | 1.7 | 2 | | |
| | | 0.63 | 86.2 | 4.3 | 2 | | |
| | | 0.31 | 87 | 0.9 | 2 | | |
| | | 0.16 | 77.2 | 0.9 | 2 | | |
| | | 0.08 | 63 | 2.6 | 2 | | |
| | | 0.04 | 43.2 | 2.7 | 2 | | |
| | COMPOSITION | 5 | 57.6 | 1 | 2 | 58.6 | 0.12 |
| | 24 | 2.5 | 60.5 | 9.6 | 2 | | |
| | | 1.25 | 52.6 | 0.6 | 2 | | |
| | | 0.63 | 59.7 | 0.6 | 2 | | |
| | | 0.31 | 46.5 | 1.8 | 2 | | |
| | | 0.16 | 35.9 | 7.6 | 2 | | |
| | | 0.08 | 22.1 | 11.2 | 2 | | |
| | | 0.04 | 10.2 | 2 | 2 | | |

### Example 5. Editing in NK Cells

5.1. LNP Formulations

LNPs were formulated as described in Example 1 except cryo-electron microscopy was not performed for LNP characterization. LNPs were formulated with a lipid amine to RNA phosphate (N:P) molar ratio of about 6, a ratio of sgRNA to Cas9 mRNA (cargo ratio) at 1:2 by weight for Compositions 26 and 27 or 1:1 cargo ratio by weight for Composition 25, and Compound 3 or Compound 8 (heptadecan-9-yl 8-((2-hydroxyethyl)(8-(nonyloxy)-8-oxooctyl)amino)octanoate), as shown in Table 10. LNPs delivered mRNA encoding Cas9 (SEQ ID No. 4) and sgRNA (SEQ ID NO. 11) targeting human AAVS1 gene.

Lipid components in LNPs were analyzed quantitatively by HPLC coupled to a charged aerosol detector (CAD). Chromatographic separation of 4 lipid components was achieved by reverse phase HPLC. HPLC lipid analysis provided the actual molar percent (mol-%) lipid levels for each component of the LNP formulations described in the following examples as shown in Table 10.

TABLE 10
Results of lipid analysis for LNP compositions

| LNP | Molar ratio | Actual or measured mol % | | | |
|---|---|---|---|---|---|
| Composition | (nominal) | Lipid | DSPC | Cholesterol | PEG |
| Composition 25 | 30/10/59/1.0 | 31.0 | 10.0 | 57.8 | 1.1 |
| (Cargo 1:1; | | | | | |
| Compound 3) | | | | | |
| Composition 26 | 50/10/38.5/1.5 | 51.9 | 9.4 | 36.7 | 1.9 |
| (Cargo 1:2; | | | | | |
| Compound 3) | | | | | |
| Composition 27 | 50/10/38.5/1.5 | 50.8 | 9.5 | 37.8 | 1.9 |
| (Cargo 1:2; | | | | | |
| compound 8) | | | | | |

LNP formulations were analyzed for Z-average and number average particle size, polydispersity (pdi), total RNA content and encapsulation efficiency of RNA according to the methods described in Example 1 and results shown in Table 11.

TABLE 11
LNP formulation analysis

| | Encap- | Z-Ave | | Num | RNA | |
|---|---|---|---|---|---|---|
| LNP | sulation | Size | | Ave | Conc. | N/P |
| Composition | (%) | (nm) | PDI | Size (nm) | (mg/mL) | ratio |
| Composition 25 | 99% | 91 | 0.07 | 77 | 0.07 | 6 |
| Composition 26 | 96% | 74 | 0.06 | 57 | 0.07 | 6 |
| Composition 27 | 92% | 71 | 0.1 | 50 | 0.07 | 6 |

NK cells were isolated from a commercially obtained leukopak from a healthy donor using the EasySep Human NK Cell Isolation Kit (STEMCELL, Cat. No. 17955) according to the manufacturer's protocol. Following isolation, NK cells were stored frozen until needed. Following cell thawing, NK cells were rested overnight in CTS™ OpTmizer™ T Cell Expansion media (Gibco, Cat. No. A10221-01) with 5% human AB serum (GemCell Cat. No. 100-512), 500 U/mL IL-2 (Peprotech, Cat. No. 200-02), 5 ng/ml IL-15 (Peprotech, Cat. No. 200-15), 10 ml Glutamax (Gibco Cat. No. 35050-61), 10 ml HEPES (Gibco, Cat. No. 15630-080) and 1% penicillin-streptomycin (ThermoFisher, Cat. No. 15140-122). The rested NK cells were then cultured at 1:1 ratio with irradiated K562 cells expressing 41BBIL (SEQ ID NO: 12) and membrane bound. IL21 (SEQ ID NO: 13) were used as feeder cells for NK activation in the above CTS™ OpTmizer™ T Cell Expansion media for 3 days. Three days following activation, NK cells were treated with LNPs delivering Cas9 mRNA (SEQ ID NO: 4) and sgRNA (SEQ ID NO: 11) targeting AAVS1 locus. A 12-pt dose response curve was generated by performing a 1:2 fold serial dilution series starting with 10 μg/mL LNPs mixed with ApoE3 (Peprotech 350-02) at 2.5 μg/ml in the above CTS™ OpTmizer™ T Cell Expansion media with 2.5% human AB serum and 0.25 uM of a small molecule inhibitor of DNA-dependent protein kinase.

The DNA-dependent protein kinase inhibitor, referred to hereinafter as “DNAPKI Compound 4” is 9-(4,4-difluorocyclohexyl)-7-methyl-2-((7-methyl-[1,2,4]triazolo[1,5-a]pyridin-6-yl)amino)-7,9-dihydro-8H-purin-8-one, also depicted as:

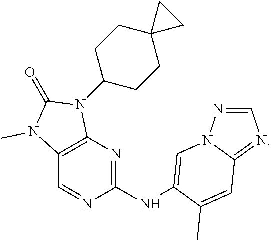 [SMILES: Cc1cc2ncnn2cc1Nc1ncc2c(n1)n(C1CCC3(CC1)CC3)c(=O)n2C]

DNAPKI Compound 4 was prepared as follows:

General Information

All reagents and solvents were purchased and used as received from commercial vendors or synthesized according to cited procedures. All intermediates and final compounds were purified using flash column chromatography on silica gel. NMR spectra were recorded on a Bruker or Varian 400 MHz spectrometer, and NMR data were collected in CDCl3 at ambient temperature. Chemical shifts are reported in parts per million (ppm) relative to CDCl3 (7.26). Data for 1H NMR are reported as follows: chemical shift, multiplicity (br=broad, s=singlet, d=doublet, t=triplet, q=quartet, dd=doublet of doublets, dt=doublet of triplets m=multiplet), coupling constant, and integration. MS data were recorded on a Waters SQD2 mass spectrometer with an electrospray ionization (ESI) source. Purity of the final compounds was determined by UPLC-MS-ELS using a Waters Acquity H-Class liquid chromatography instrument equipped with SQD2 mass spectrometer with photodiode array (PDA) and evaporative light scattering (ELS) detectors.

### Intermediate 1a: (E)-N,N-dimethyl-N′-(4-methyl-5-nitropyridin-2-yl)formimidamide

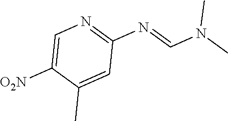

To a solution of 4-methyl-5-nitro-pyridin-2-amine (5 g, 1.0 equiv.) in toluene (0.3 M) was added DMF-DMA (3.0 equiv.). The mixture was stirred at 110° C. for 2 h. The reaction mixture was concentrated under reduced pressure to give a residue and purified by column chromatography to afford product as a yellow solid (59%). ¹H NMR (400 MHz, (CD₃)₂SO) δ 8.82 (s, 1H), 8.63 (s, 1H), 6.74 (s, 1H), 3.21 (m, 6H).

### Intermediate 1b: (E)-N-hydroxy-N′-(4-methyl-5-nitropyridin-2-yl)formimidamide

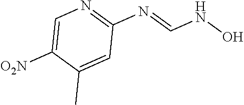

To a solution of Intermediate 1a (4 g, 1.0 equiv.) in MeOH (0.2 M) was added NH₂OH·HCl (2.0 equiv.). The reaction mixture was stirred at 80° C. for 1 h. The reaction mixture was filtered, and the filtrate was concentrated under reduced pressure to give a residue. The residue was partitioned between H₂O and EtOAc, followed by 2× extraction with EtOAc. The organic phases were concentrated under reduced pressure to give a residue and purified by column chromatography to afford product as a white solid (66%). ¹H NMR (400 MHz, (CD₃)₂SO) δ 10.52 (d, J=3.8 Hz, 1H), 10.08 (dd, J=9.9, 3.7 Hz, 1H), 8.84 (d, J=3.8 Hz, 1H), 7.85 (dd, J=9.7, 3.8 Hz, 1H), 7.01 (d, J=3.9 Hz, 1H), 3.36 (s, 3H).

### Intermediate 1c: 7-methyl-6-nitro-[1,2,4]triazolo[1,5-a]pyridine

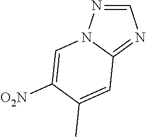

To a solution of Intermediate 1b (2.5 g, 1.0 equiv.) in THF (0.4 M) was added trifluoroacetic anhydride (1.0 equiv.) at 0° C. The mixture was stirred at 25° C. for 18 h. The reaction mixture was filtered, and the filtrate was concentrated under reduced pressure to give a residue. The residue was purified by column chromatography to afford product as a white solid (44%). ¹H NMR (400 MHz, CDCl₃) δ 9.53 (s, 1H), 8.49 (s, 1H), 7.69 (s, 1H), 2.78 (d, J=1.0 Hz, 3H).

### Intermediate 1d: 7-methyl-[1,2,4]triazolo[1,5-a]pyridin-6-amine

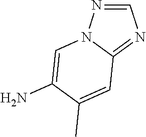

To a mixture of Pd/C (10% w/w, 0.2 equiv.) in EtOH (0.1 M) was added Intermediate 1c (1.0 equiv. and ammonium formate (5.0 equiv.). The mixture was heated at 105° C. for 2 h. The reaction mixture was filtered, and the filtrate was concentrated under reduced pressure to give a residue. The residue was purified by column chromatography to afford product as a pale brown solid. ¹H NMR (400 MHz, (CD₃)₂SO) δ 8.41 (s, 2H), 8.07 (d, J=9.0 Hz, 2H), 7.43 (s, 1H), 2.22 (s, 3H).

### Intermediate 1e: 8-methylene-1,4-dioxaspiro[4.5]decane

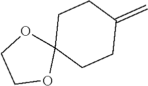

To a solution of methyl(triphenyl)phosphonium bromide (1.15 equiv.) in THF (0.6 M) was added n-BuLi (1.1 equiv.) at −78° C. dropwise, and the mixture was stirred at 0° C. for 1 h. Then, 1,4-dioxaspiro[4.5]decan-8-one (50 g, 1.0 equiv.) was added to the reaction mixture. The mixture was stirred at 25° C. for 12 h. The reaction mixture was poured into aq. NH₄Cl at 0° C., diluted with H₂O, and extracted 3× with EtOAc. The combined organic layers were concentrated under reduced pressure to give a residue and purified by column chromatography to afford product as a colorless oil (51%). ¹H NMR (400 MHz, CDCl₃) δ 4.67 (s, 1H), 3.96 (s, 4H), 2.82 (t, J=6.4 Hz, 4H), 1.70 (t, J=6.4 Hz, 4H).

### Intermediate 1f: 7,10-dioxadispiro[2.2.4⁶.2³]dodecane

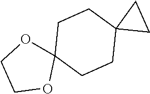 [SMILES: C1COC2(CCC3(CC3)CC2)O1]

To a solution of Intermediate 4a (5 g, 1.0 equiv.) in toluene (3 M) was added ZnEt₂ (2.57 equiv.) dropwise at −40° C. and the mixture was stirred at −40° C. for 1 h. Then diiodomethane (6.0 equiv.) was added dropwise to the mixture at −40° C. under N₂. The mixture was then stirred at 20° C. for 17 h under N₂ atmosphere. The reaction mixture was poured into aq. NH₄Cl at 0° C. and extracted 2× with EtOAc. The combined organic phases were washed with brine (20 mL), dried with anhydrous Na₂SO₄, filtered, and the filtrate was concentrated in vacuum. The residue was purified by column chromatography to afford product as a pale yellow oil (73%).

### Intermediate 1g: spiro[2.5]octan-6-one

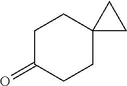

To a solution of Intermediate 4b (4 g, 1.0 equiv.) in 1:1 THF/H₂O (1.0 M) was added TFA (3.0 equiv.). The mixture was stirred at 20° C. for 2 h under N₂ atmosphere. The reaction mixture was concentrated under reduced pressure to remove THF, and the residue adjusted pH to 7 with 2 M NaOH (aq.). The mixture was poured into water and 3× extracted with EtOAc. The combined organic phase was washed with brine, dried with anhydrous Na₂SO₄, filtered, and the filtrate was concentrated in vacuum. The residue was purified by column chromatography to afford product as a pale yellow oil (68%). ¹H NMR (400 MHz, CDCl₃) δ 2.35 (t, J=6.6 Hz, 4H), 1.62 (t, J=6.6 Hz, 4H), 0.42 (s, 4H).

### Intermediate 1h: N-(4-methoxybenzyl)spiro[2.5]octan-6-amine

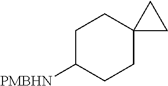

To a mixture of Intermediate 4c (2 g, 1.0 equiv.) and (4-methoxyphenyl)methanamine (1.1 equiv.) in DCM (0.3 M) was added AcOH (1.3 equiv.). The mixture was stirred at 20° C. for 1 h under N₂ atmosphere. Then, NaBH(OAc)₃ (3.3 equiv.) was added to the mixture at 0° C., and the mixture was stirred at 20° C. for 17 h under N₂ atmosphere. The reaction mixture was concentrated under reduced pressure to remove DCM, and the resulting residue was diluted with H₂O and extracted 3× with EtOAc. The combined organic layers were washed with brine, dried over Na₂SO₄, filtered, and the filtrate was concentrated under reduced pressure to give a residue. The residue was purified by column chromatography to afford product as a gray solid (51%). ¹H NMR (400 MHz, (CD₃)₂SO) δ 7.15-7.07 (m, 2H), 6.77-6.68 (m, 2H), 3.58 (s, 3H), 3.54 (s, 2H), 2.30 (ddt, J=10.1, 7.3, 3.7 Hz, 1H), 1.69-1.62 (m, 2H), 1.37 (td, J=12.6, 3.5 Hz, 2H), 1.12-1.02 (m, 2H), 0.87-0.78 (m, 2H), 0.13-0.04 (m, 2H).

### Intermediate 1i: spiro[2.5]octan-6-amine

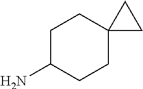

To a suspension of Pd/C (10% w/w, 1.0 equiv.) in MeOH (0.25 M) was added Intermediate 4d (2 g, 1.0 equiv.) and the mixture was stirred at 80° C. at 50 Psi for 24 h under H₂ atmosphere. The reaction mixture was filtered, and the filtrate was concentrated under reduced pressure to give a residue that was purified by column chromatography to afford product as a white solid. ¹H NMR (400 MHz, (CD₃)₂SO) δ 2.61 (tt, J=10.8, 3.9 Hz, 1H), 1.63 (ddd, J=9.6, 5.1, 2.2 Hz, 2H), 1.47 (td, J=12.8, 3.5 Hz, 2H), 1.21-1.06 (m, 2H), 0.82-0.72 (m, 2H), 0.14-0.05 (m, 2H).

### Intermediate 1j: ethyl 2-chloro-4-(spiro[2.5]octan-6-ylamino)pyrimidine-5-carboxylate

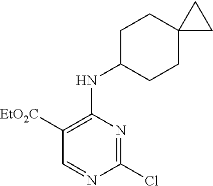 [SMILES: CCOC(=O)c1cnc(Cl)nc1NC1CCC2(CC1)CC2]

To a mixture of ethyl 2,4-dichloropyrimidine-5-carboxylate (2.7 g, 1.0 equiv.) and Intermediate 1i (1.0 equiv.) in ACN (0.5-0.6 M) was added K₂CO₃ (2.5 equiv.) in one portion under N₂. The mixture was stirred at 20° C. for 12 h. The reaction mixture was filtered, and the filtrate was concentrated under reduced pressure to give a residue. The residue was purified by column chromatography to afford product as a white solid (54%). ¹H NMR (400 MHz, (CD₃)₂SO) δ 8.64 (s, 1H), 8.41 (d, J=7.9 Hz, 1H), 4.33 (q, J=7.1 Hz, 2H), 4.08 (d, J=9.8 Hz, 1H), 1.90 (dd, J=12.7, 4.8 Hz, 2H), 1.64 (t, J=12.3 Hz, 2H), 1.52 (q, J=10.7, 9.1 Hz, 2H), 1.33 (t, J=7.1 Hz, 3H), 1.12 (d, J=13.0 Hz, 2H), 0.40-0.21 (m, 4H).

### Intermediate 1k: 2-chloro-4-(spiro[2.5]octan-6-ylamino)pyrimidine-5-carboxylic acid

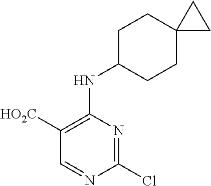 [SMILES: O=C(O)c1cnc(Cl)nc1NC1CCC2(CC1)CC2]

To a solution of Intermediate 1j (2 g, 1.0 equiv.) in 1:1 THF/H₂O (0.3 M) was added LiOH (2.0 equiv.). The mixture was stirred at 20° C. for 12 h. The reaction mixture was filtered, and the filtrate was concentrated under reduced pressure to give a residue. The residue was adjusted to pH 2 with 2 M HCl, and the precipitate was collected by filtration, washed with water, and tried under vacuum. Product was used directly in the next step without additional purification (82%). ¹H NMR (400 MHz, (CD₃)₂SO) δ 13.54 (s, 1H), 8.38 (d, J=8.0 Hz, 1H), 8.35 (s, 1H), 3.82 (qt, J=8.2, 3.7 Hz, 1H), 1.66 (dq, J=12.8, 4.1 Hz, 2H), 1.47-1.34 (m, 2H), 1.33-1.20 (m, 2H), 0.86 (dt, J=13.6, 4.2 Hz, 2H), 0.08 (dd, J=8.3, 4.8 Hz, 4H).

### Intermediate 1l: 2-chloro-9-(spiro[2.5]octan-6-yl)-7,9-dihydro-8H-purin-8-one

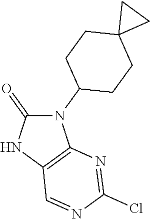 [SMILES: O=c1[nH]c2cnc(Cl)nc2n1C1CCC2(CC1)CC2]

To a mixture of Intermediate 1k (1.5 g, 1.0 equiv.) and Et₃N (1.0 equiv.) in DMF (0.3 M) was added DPPA (1.0 equiv.). The mixture was stirred at 120° C. for 8 h under N₂ atmosphere. The reaction mixture was poured into water. The precipitate was collected by filtration, washed with water, and dried under vacuum to give a residue that was used directly in the next step without additional purification (67%). ¹H NMR (400 MHz, (CD₃)₂SO) δ 11.68 (s, 1H), 8.18 (s, 1H), 4.26 (ddt, J=12.3, 7.5, 3.7 Hz, 1H), 2.42 (qd, J=12.6, 3.7 Hz, 2H), 1.95 (td, J=13.3, 3.5 Hz, 2H), 1.82-1.69 (m, 2H), 1.08-0.95 (m, 2H), 0.39 (tdq, J=11.6, 8.7, 4.2, 3.5 Hz, 4H).

### Intermediate 1m: 2-chloro-7-methyl-9-(spiro[2.5]octan-6-yl)-7,9-dihydro-8H-purin-8-one

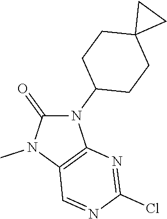 [SMILES: Cn1c(=O)n(C2CCC3(CC2)CC3)c2nc(Cl)ncc21]

To a mixture of Intermediate 11 (1.0 g, 1.0 equiv.) and NaOH (5.0 equiv.) in 1:1 THF/H₂O (0.3-0.5 M) was added MeI (2.0 equiv.). The mixture was stirred at 20° C. for 12 h under N₂ atmosphere. The reaction mixture was concentrated under reduced pressure to afford a residue that was purified by column chromatography to afford product as a pale yellow solid (67%). ¹H NMR (400 MHz, CDCl₃) δ 7.57 (s, 1H), 4.03 (tt, J=12.5, 3.9 Hz, 1H), 3.03 (s, 3H), 2.17 (qd, J=12.6, 3.8 Hz, 2H), 1.60 (td, J=13.4, 3.6 Hz, 2H), 1.47-1.34 (m, 2H), 1.07 (s, 1H), 0.63 (dp, J=14.0, 2.5 Hz, 2H), −0.05 (s, 4H).

### DNAPKI Compound 4: 7-methyl-2-((7-methyl-[1,2,4]triazolo[1,5-a]pyridin-6-yl)amino)-9-(spiro[2.5]octan-6-yl)-7,9-dihydro-8H-purin-8-one

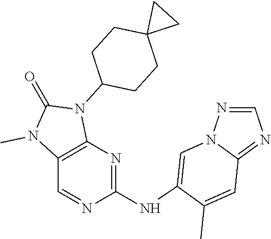 [SMILES: Cc1cc2ncnn2cc1Nc1ncc2c(n1)n(C1CCC3(CC1)CC3)c(=O)n2C]

To a mixture of Intermediate 1m (1.0 equiv.) and Intermediate 1d (1.0 equiv.), Pd(dppf)C₁₂ (0.2 equiv.), XantPhos (0.4 equiv.), and Cs₂CO₃ (2.0 equiv.) in DMF (0.2-0.3 M) was degassed and purged 3× with N₂, and the mixture was stirred at 130° C. for 12 h under N₂ atmosphere. The mixture was then poured into water and extracted 3× with DCM. The combined organic phase was washed with brine, dried over Na₂SO₄, filtered, and the filtrate was concentrated in vacuum. The residue was purified by column chromatography to afford product as an off-white solid. ¹H NMR (400 MHz, (CD₃)₂SO) δ 9.09 (s, 1H), 8.73 (s, 1H), 8.44 (s, 1H), 8.16 (s, 1H), 7.78 (s, 1H), 4.21 (t, J=12.5 Hz, 1H), 3.36 (s, 3H), 2.43 (s, 3H), 2.34 (dt, J=13.0, 6.5 Hz, 2H), 1.93-1.77 (m, 2H), 1.77-1.62 (m, 2H), 0.91 (d, J=13.2 Hz, 2H), 0.31 (t, J=7.1 Hz, 2H). MS: 405.5 m/z [M+H].

The final concentrations of total RNA cargo in LNPs were 10, 5, 2.5, 1.25, 0.63, 0.31, 0.16, 0.08, 0.04, 0.02, 0.01, 0.005, and 0 μg/ml (untreated controls) as indicated in Table 12. The mixed LNPs were added to NK cells of 1×10⁶ cells/ml at 1:1 ratio in triplicate. Seven days post LNP treatment, genomic DNA was isolated from cells and NGS analysis performed as described in Example 1.

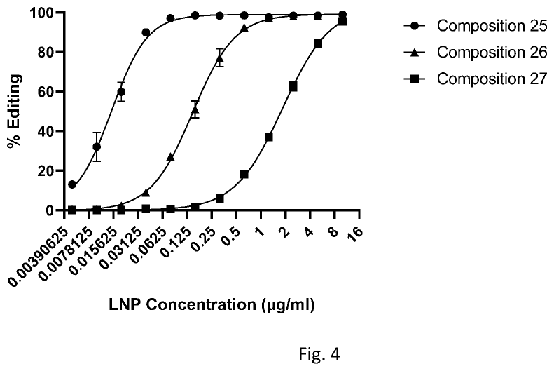

Mean percent editing, standard deviation, and EC50s of LNP formulations at the indicated concentrations are shown in Table 12 and dose response curves in FIG. 4.

TABLE 12
Mean percent editing in NK cells

| | Composition | | Composition | | Composition | |
|---|---|---|---|---|---|---|
| LNP | 26 | | 25 | | 27 | |
| (μg/mL) | Mean | SD | Mean | SD | Mean | SD |
| 10 | 98.8 | 0.5 | 98.9 | 0.1 | 95.4 | 0.4 |
| 5 | 98.4 | 0.2 | 98.4 | 0.1 | 84.3 | 2.2 |
| 2.5 | 98.0 | 0.2 | 98.4 | 0.2 | 62.7 | 2.3 |
| 1.25 | 97.2 | 0.4 | 97.6 | 0.6 | 36.9 | 1.4 |
| 0.63 | 92.5 | 1.4 | 98.6 | 0.2 | 18.1 | 1.0 |
| 0.31 | 77.1 | 4.5 | 98.4 | 0.2 | 6.0 | 0.7 |
| 0.16 | 51.0 | 4.3 | 98.5 | 0.0 | 1.8 | 0.4 |
| 0.08 | 27.1 | 1.3 | 97.1 | 0.2 | 0.6 | 0.2 |
| 0.04 | 8.8 | 0.3 | 90.0 | 1.4 | 0.8 | 0.5 |
| 0.02 | 2.3 | 0.2 | 59.9 | 4.8 | 0.1 | 0.1 |
| 0.01 | 0.6 | 0.1 | 32.0 | 7.3 | 0.1 | 0.0 |
| 0.005 | 0.4 | 0.1 | 13.1 | 1.9 | 0.1 | 0.0 |
| 0 | 0.2 | 0.1 | 0.1 | 0.0 | 0.3 | 0.3 |

### Example 6. Monocyte and Macrophage Editing

CD14+ cells were isolated from a leukopak obtained commercially (Hemacare) using StraightFrom® Leukopak® CD14 MicroBead Kit, human (Miltenyi Biotec, Catalog, 130-117-020) following the manufacturers protocol on MultiMACS™ Ce1124 Separator Plus instrument (Miltenyi Biotec). CD14+ cells were thawed and cultured in triplicate at 50,000 cells/well in OpTmizer base media as described in Example 1 with 10 ng/mL GM-CSF (Stemcell, 78140.1) at a cell density of 1 million/mL on 96-well non-tissue culture plates (Falcon, 351172). Every 2-3 days, 50% of the OpTmizer media per well was replaced with ng/mL of fresh cytokine media (GM-CSF (Stemcell, 78140.1), 2.5% HS OpTmizer (Gibco, A3705001).

Cells were treated with LNPs prepared as described in Example 10. LNPs were preincubated at 37° C. for 15 minutes with ApoE3 (Peprotech 350-02) at 10 μg/ml. The pre-incubated LNPs were added to cells in 1:1 v/v ratio, yielding a final total RNA cargo dose of 0-1.25 μg/mL.

Post incubation, 50 μL of the LNP concentrations were added to monocytes the same day CD14+ cells were plated on non-tissue culture plates (Falcon, 351172) and to macrophages after 5 days of incubation on non-tissue culture plates (Falcon, 351172). Monocyte and macrophage plates were incubated at 37° C. until use.

Six days post LNP treatment, genomic DNA was isolated as described in Example 1 from the monocyte and macrophage-engineered cells were collected for NGS as described in Example 1.

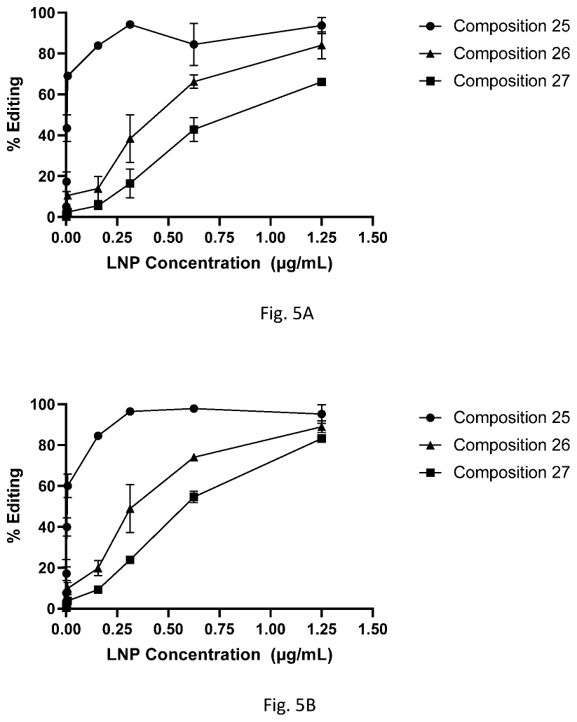

Mean percent editing, standard deviation, and EC50 of each LNP formulation at the indicated concentrations are shown in Table 13 for monocytes and Table 14 for macrophages. Dose response curves for monocytes and macrophages are shown in FIGS. 5A and 5B, respectively.

TABLE 13
Mean percent editing six days after treatment of monocytes
with LNPs with varied ionizable lipids

| LNP con- | | | | | | | | | |
|---|---|---|---|---|---|---|---|---|---|
| centration | Composition 25 | | | Composition 26 | | | Composition 27 | | |
| (ng/ml) | Mean | SD | EC50 | Mean | SD | EC50 | Mean | SD | EC50 |
| 0 | 0.1 | 0 | 42.1 | 0.1 | 0 | 407 | 0.2 | 0.1 | 587 |
| 4.88 | 2.5 | 0.9 | | 0.2 | 0 | | 0.1 | 0 | |
| 9.77 | 5.1 | 1.4 | | 0.5 | 0.3 | | 0.2 | 0.1 | |
| 19.53 | 17.3 | 4.8 | | 0.6 | 0.5 | | 0.2 | 0.1 | |
| 39.06 | 43.5 | 6.5 | | 2.6 | 0.3 | | 0.5 | 0.1 | |
| 78.13 | 69.1 | 1.9 | | 10.5 | 1.3 | | 2.4 | 0.1 | |
| 156.25 | 83.9 | 1.5 | | 13.9 | 6 | | 5.5 | 0.9 | |
| 312.5 | 94.2 | 0.1 | | 38.4 | 11.7 | | 16.4 | 7 | |
| 625 | 84.5 | 10.3 | | 66.2 | 3.2 | | 42.9 | 5.9 | |
| 1250 | 93.7 | 4.0 | | 84.0 | 6.7 | | 66.1 | 0.8 | |
| 2500 | 95.0 | 3.0 | | 95.0 | 1.5 | | 84.0 | 2.1 | |
| 5000 | 97.0 | 0.4 | | 97.0 | 1.4 | | 86.2 | 4.2 | |

TABLE 14
Mean percent editing six days after treatment of macrophages
with LNPs with varied ionizable lipids

| LNP | | | | | | | | | |
|---|---|---|---|---|---|---|---|---|---|
| concentration | Composition 25 | | | Composition 26 | | | Composition 27 | | |
| (ng/ml) | Mean | SD | EC50 | Mean | SD | EC50 | Mean | SD | EC50 |
| 0 | 2.2 | 1.1 | 54.9 | 1.6 | 0.9 | 745 | 0.3 | 0.1 | 613 |
| 4.88 | 3.3 | 0.7 | | 0.3 | 0.2 | | 0.4 | 0.1 | |
| 9.77 | 7.6 | 1.7 | | 9.3 | 14.8 | | 0.7 | 0.3 | |
| 19.53 | 17.1 | 3.3 | | 1.6 | 0.8 | | 1.1 | 0.7 | |
| 39.06 | 40 | 4.6 | | 3.4 | 0.5 | | 1.5 | 0.3 | |
| 78.13 | 60 | 5.7 | | 9.8 | 3 | | 3.8 | 0.9 | |
| 156.25 | 84.5 | 1.4 | | 19.8 | 3.7 | | 9.3 | 0.7 | |
| 312.5 | 96.4 | 0.3 | | 48.9 | 11.8 | | 23.9 | 1.3 | |
| 625 | 97.9 | 1.8 | | 74.0 | 0.1 | | 54.6 | 2.8 | |
| | | | | *n = 2 | | | | | |
| 1250 | 95.2 | 4.6 | | 89 | 2.8 | | 83.2 | 1.5 | |
| 2500 | 98.2 | 0.4 | | 96.2 | 0.7 | | 86.7 | 3.6 | |
| 5000 | 95.3 | 1.4 | | 59.7 | 2.2 | | 48.6 | 5.6 | |

### Example 7. B Cell Editing

7.1. B Cell Isolation and Culture and Media Preparation

B cells (Hemacare) were cultured in Stemspan SFEM media (StemCell Technologies, cat. 09650) supplemented with 1% penicillin-streptomycin (ThermoFisher, cat. 15140122), 1 μg/ml CpG ODN 2006 (Invivogen, cat. tlrl-2006-1), 50 ng/ml IL-2 (Peprotech, cat. 200-02), 50 ng/ml IL-10 (Peprotech, cat. 200-10) and 10 ng/ml IL-15 (Peprotech, cat. 200-15). Two media components of variable concentrations were also used to supplement the media: 1. Human Serum AB (Gemini Bioproducts, cat.100-512, lot #H94X00K, 2.5% and 5%) and 2. MEGACD40L (Enzo Life Sciences, cat. ALX-522-110-0000, 1 ng/ml and 100 ng/ml). B cell culture media compositions used for preparing B cells are described in Table 15.

TABLE 15
B cell media compositions

| | |
|---|---|
| Media | |
| Number | Media Composition |

| | | |
|---|---|---|
| 1 | StemSpan SFEM media + | With 5% |
| | Pen/Strep, IL2, IL10, | Human Serum |
| | IL15, CpG ODN, | AB |
| | 1 ng/ml MEGACD40L | |
| 2 | StemSpan SFEM media + | With 2.5% |
| | Pen/Strep, IL2, IL10, | Human Serum |
| | IL15, CpG ODN, | AB |
| | 1 ng/ml MEGACD40L | |
| 3 | StemSpan SFEM media + | With 5% |
| | Pen/Strep, IL2, IL10, | Human Serum |
| | IL15, CpG ODN, | AB |
| | 100 ng/ml MEGACD40L | |

B cells were isolated by CD19 positive selection from a leukopak from a healthy human donor (Hemacare) using the StraightFrom Leukopak CD19 MicroBead kit (Miltenyi, 130-117-021) on a MultiMACS Ce1124 Separator Plus instrument according to the manufacturer's instructions. Isolated CD19+ B cells were stored frozen in liquid nitrogen until needed.

When ready for use, B cells were thawed and activated the same day in B Cell Media 1. Two days following B cell thawing and activation, B cells were cultured in Media 2 and treated with LNPs delivering Cas9 mRNA (SEQ ID NO: 4) and gRNA (SEQ ID NO: 11) targeting AAVS1. Several concentrations were tested for each LNP to generate an 8-point dose response curve by setting up a 1:2 serial dilution, starting at 20 μg/ml total RNA cargo (4× the final dose). Subsequently, 4 μg/ml ApoE3 in B Cell Media 2 was added (4× the final dose), before adding the B cells at a 1:1 ratio v/v to the LNP-APOE3 mixture, resulting in a final dose of total RNA cargo of 5, 2.5, 1.25, 0.625, 0.313, 0.156, 0.078 μg/ml as indicated in Table 16. Cells were treated with LNPs prepared and analyzed as described in Example or not treated with LNPs to serve as controls.

Three days post LNP treatment, cells were washed and resuspended in B Cell Media 3. Seven days post LNP treatment, cells were collected and NGS analysis was performed as described in Example 1.

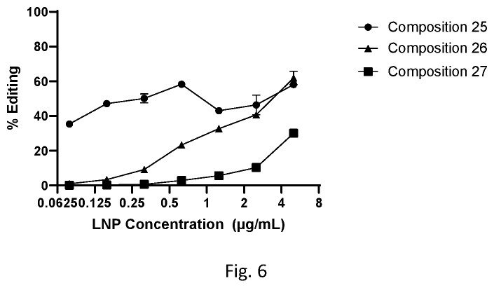

Mean percent editing and standard deviation of the LNP formulations at the indicated concentrations is shown in Table 16 and dose response curves in FIG. 6. “Untreated B cells” were not treated with the LNP formulation.

TABLE 16
Mean percent editing in B cells following
editing with described lipid compositions

| | LNP conc. | | |
|---|---|---|---|
| Composition | (ng/mL) | Mean | SD |
| Untreated B cells | | 0.1 | 0.1 |
| Composition 25 | 5 | 58.2 | 0.6 |
| | 2.5 | 46.4 | 5.7 |
| | 1.25 | 43.1 | 1.2 |
| | 0.625 | 58.3 | 0.4 |
| | 0.313 | 50.2 | 2.6 |
| | 0.156 | 47.1 | 0.0 |
| | 0.078 | 35.4 | 1.1 |
| Untreated B cells | 0 | 0.1 | 0.0 |
| Composition 26 | 5 | 61.9 | 3.8 |
| | 2.5 | 40.7 | 0.4 |
| | 1.25 | 32.7 | 0.7 |
| | 0.625 | 23.3 | 0.9 |
| | 0.313 | 9.2 | 0.4 |
| | 0.156 | 3.4 | 0.5 |
| | 0.078 | 1.0 | 0.1 |
| Untreated B cells | 0 | 0.1 | 0.0 |
| Composition 27 | 5 | 30.0 | 0.7 |
| | 2.5 | 10.3 | 0.0 |
| | 1.25 | 5.7 | 1.2 |
| | 0.625 | 2.9 | 0.6 |
| | 0.313 | 0.7 | 0.2 |
| | 0.156 | 0.3 | 0.1 |
| | 0.078 | 0.1 | 0.0 |

In the following table and throughout, the terms “mA,” “mC,” “mU,” or “mG” are used to denote a nucleotide that has been modified with 2′-O-Me.

In the following table, a “*” is used to depict a PS modification. In this application, the terms A*, C*, U*, or G* may be used to denote a nucleotide that is linked to the next (e.g., 3′) nucleotide with a PS bond.

It is understood that if a DNA sequence (comprising Ts) is referenced with respect to an RNA, then Ts should be replaced with Us (which may be modified or unmodified depending on the context), and vice versa.

In the following table, single amino acid letter code is used to provide peptide sequences.

TABLE 17
List of sequences

| | SEQ ID | |
|---|---|---|
| Description | NO | Sequence |
| ORF | SEQ ID | ATGGACAAGAAGTACAGCATCGGACTGGACATCGGAACAAACAGCGTCGGATG |
| encoding | NO: 1 | GGCAGTCATCACAGACGAATACAAGGTCCCGAGCAAGAAGTTCAAGGTCCTGG |
| Sp. Cas9 | | GAAACACAGACAGACACAGCATCAAGAAGAACCTGATCGGAGCACTGCTGTTC |
| | | GACAGCGGAGAAACAGCAGAAGCAACAAGACTGAAGAGAACAGCAAGAAGAAG |
| | | ATACACAAGAAGAAAGAACAGAATCTGCTACCTGCAGGAAATCTTCAGCAACG |
| | | AAATGGCAAAGGTCGACGACAGCTTCTTCCACAGACTGGAAGAAAGCTTCCTG |
| | | GTCGAAGAAGACAAGAAGCACGAAAGACACCCGATCTTCGGAAACATCGTCGA |
| | | CGAAGTCGCATACCACGAAAAGTACCCGACAATCTACCACCTGAGAAAGAAGC |
| | | TGGTCGACAGCACAGACAAGGCAGACCTGAGACTGATCTACCTGGCACTGGCAC |
| | | ACATGATCAAGTTCAGAGGACACTTCCTGATCGAAGGAGACCTGAACCCGGAC |
| | | AACAGCGACGTCGACAAGCTGTTCATCCAGCTGGTCCAGACATACAACCAGCTG |
| | | TTCGAAGAAAACCCGATCAACGCAAGCGGAGTCGACGCAAAGGCAATCCTGAG |
| | | CGCAAGACTGAGCAAGAGCAGAAGACTGGAAAACCTGATCGCACAGCTGCCGG |
| | | GAGAAAAGAAGAACGGACTGTTCGGAAACCTGATCGCACTGAGCCTGGGACTG |
| | | ACACCGAACTTCAAGAGCAACTTCGACCTGGCAGAAGACGCAAAGCTGCAGCTG |
| | | AGCAAGGACACATACGACGACGACCTGGACAACCTGCTGGCACAGATCGGAGA |
| | | CCAGTACGCAGACCTGTTCCTGGCAGCAAAGAACCTGAGCGACGCAATCCTGCT |
| | | GAGCGACATCCTGAGAGTCAACACAGAAATCACAAAGGCACCGCTGAGCGCAA |
| | | GCATGATCAAGAGATACGACGAACACCACCAGGACCTGACACTGCTGAAGGCA |
| | | CTGGTCAGACAGCAGCTGCCGGAAAAGTACAAGGAAATCTTCTTCGACCAGAG |
| | | CAAGAACGGATACGCAGGATACATCGACGGAGGAGCAAGCCAGGAAGAATTCT |
| | | ACAAGTTCATCAAGCCGATCCTGGAAAAGATGGACGGAACAGAAGAACTGCTG |
| | | GTCAAGCTGAACAGAGAAGACCTGCTGAGAAAGCAGAGAACATTCGACAACGG |
| | | AAGCATCCCGCACCAGATCCACCTGGGAGAACTGCACGCAATCCTGAGAAGACA |
| | | GGAAGACTTCTACCCGTTCCTGAAGGACAACAGAGAAAAGATCGAAAAGATCC |
| | | TGACATTCAGAATCCCGTACTACGTCGGACCGCTGGCAAGAGGAAACAGCAGA |
| | | TTCGCATGGATGACAAGAAAGAGCGAAGAAACAATCACACCGTGGAACTTCGA |
| | | AGAAGTCGTCGACAAGGGAGCAAGCGCACAGAGCTTCATCGAAAGAATGACAA |
| | | ACTTCGACAAGAACCTGCCGAACGAAAAGGTCCTGCCGAAGCACAGCCTGCTGT |
| | | ACGAATACTTCACAGTCTACAACGAACTGACAAAGGTCAAGTACGTCACAGAA |
| | | GGAATGAGAAAGCCGGCATTCCTGAGCGGAGAACAGAAGAAGGCAATCGTCGA |
| | | CCTGCTGTTCAAGACAAACAGAAAGGTCACAGTCAAGCAGCTGAAGGAAGACT |
| | | ACTTCAAGAAGATCGAATGCTTCGACAGCGTCGAAATCAGCGGAGTCGAAGAC |
| | | AGATTCAACGCAAGCCTGGGAACATACCACGACCTGCTGAAGATCATCAAGGA |
| | | CAAGGACTTCCTGGACAACGAAGAAAACGAAGACATCCTGGAAGACATCGTCC |
| | | TGACACTGACACTGTTCGAAGACAGAGAAATGATCGAAGAAAGACTGAAGACA |
| | | TACGCACACCTGTTCGACGACAAGGTCATGAAGCAGCTGAAGAGAAGAAGATA |
| | | CACAGGATGGGGAAGACTGAGCAGAAAGCTGATCAACGGAATCAGAGACAAGC |
| | | AGAGCGGAAAGACAATCCTGGACTTCCTGAAGAGCGACGGATTCGCAAACAGA |
| | | AACTTCATGCAGCTGATCCACGACGACAGCCTGACATTCAAGGAAGACATCCAG |
| | | AAGGCACAGGTCAGCGGACAGGGAGACAGCCTGCACGAACACATCGCAAACCT |
| | | GGCAGGAAGCCCGGCAATCAAGAAGGGAATCCTGCAGACAGTCAAGGTCGTCG |
| | | ACGAACTGGTCAAGGTCATGGGAAGACACAAGCCGGAAAACATCGTCATCGAA |
| | | ATGGCAAGAGAAAACCAGACAACACAGAAGGGACAGAAGAACAGCAGAGAAAG |
| | | AATGAAGAGAATCGAAGAAGGAATCAAGGAACTGGGAAGCCAGATCCTGAAGG |
| | | AACACCCGGTCGAAAACACACAGCTGCAGAACGAAAAGCTGTACCTGTACTACC |
| | | TGCAGAACGGAAGAGACATGTACGTCGACCAGGAACTGGACATCAACAGACTG |
| | | AGCGACTACGACGTCGACCACATCGTCCCGCAGAGCTTCCTGAAGGACGACAGC |
| | | ATCGACAACAAGGTCCTGACAAGAAGCGACAAGAACAGAGGAAAGAGCGACAA |
| | | CGTCCCGAGCGAAGAAGTCGTCAAGAAGATGAAGAACTACTGGAGACAGCTGC |
| | | TGAACGCAAAGCTGATCACACAGAGAAAGTTCGACAACCTGACAAAGGCAGAG |
| | | AGAGGAGGACTGAGCGAACTGGACAAGGCAGGATTCATCAAGAGACAGCTGGT |
| | | CGAAACAAGACAGATCACAAAGCACGTCGCACAGATCCTGGACAGCAGAATGA |
| | | ACACAAAGTACGACGAAAACGACAAGCTGATCAGAGAAGTCAAGGTCATCACA |
| | | CTGAAGAGCAAGCTGGTCAGCGACTTCAGAAAGGACTTCCAGTTCTACAAGGT |
| | | CAGAGAAATCAACAACTACCACCACGCACACGACGCATACCTGAACGCAGTCGT |
| | | CGGAACAGCACTGATCAAGAAGTACCCGAAGCTGGAAAGCGAATTCGTCTACG |
| | | GAGACTACAAGGTCTACGACGTCAGAAAGATGATCGCAAAGAGCGAACAGGAA |
| | | ATCGGAAAGGCAACAGCAAAGTACTTCTTCTACAGCAACATCATGAACTTCTTC |
| | | AAGACAGAAATCACACTGGCAAACGGAGAAATCAGAAAGAGACCGCTGATCGA |
| | | AACAAACGGAGAAACAGGAGAAATCGTCTGGGACAAGGGAAGAGACTTCGCAA |
| | | CAGTCAGAAAGGTCCTGAGCATGCCGCAGGTCAACATCGTCAAGAAGACAGAA |
| | | GTCCAGACAGGAGGATTCAGCAAGGAAAGCATCCTGCCGAAGAGAAACAGCGA |
| | | CAAGCTGATCGCAAGAAAGAAGGACTGGGACCCGAAGAAGTACGGAGGATTCG |
| | | ACAGCCCGACAGTCGCATACAGCGTCCTGGTCGTCGCAAAGGTCGAAAAGGGAA |
| | | AGAGCAAGAAGCTGAAGAGCGTCAAGGAACTGCTGGGAATCACAATCATGGAA |
| | | AGAAGCAGCTTCGAAAAGAACCCGATCGACTTCCTGGAAGCAAAGGGATACAA |
| | | GGAAGTCAAGAAGGACCTGATCATCAAGCTGCCGAAGTACAGCCTGTTCGAAC |
| | | TGGAAAACGGAAGAAAGAGAATGCTGGCAAGCGCAGGAGAACTGCAGAAGGGA |
| | | AACGAACTGGCACTGCCGAGCAAGTACGTCAACTTCCTGTACCTGGCAAGCCAC |
| | | TACGAAAAGCTGAAGGGAAGCCCGGAAGACAACGAACAGAAGCAGCTGTTCGT |
| | | CGAACAGCACAAGCACTACCTGGACGAAATCATCGAACAGATCAGCGAATTCA |
| | | GCAAGAGAGTCATCCTGGCAGACGCAAACCTGGACAAGGTCCTGAGCGCATAC |
| | | AACAAGCACAGAGACAAGCCGATCAGAGAACAGGCAGAAAACATCATCCACCT |
| | | GTTCACACTGACAAACCTGGGAGCACCGGCAGCATTCAAGTACTTCGACACAAC |
| | | AATCGACAGAAAGAGATACACAAGCACAAAGGAAGTCCTGGACGCAACACTGA |
| | | TCCACCAGAGCATCACAGGACTGTACGAAACAAGAATCGACCTGAGCCAGCTGG |
| | | GAGGAGACGGAGGAGGAAGCCCGAAGAAGAAGAGAAAGGTCTAG |
| ORF | SEQ ID | ATGGACAAGAAGTACTCCATCGGCCTGGACATCGGCACCAACTCCGTGGGCTGG |
| encoding | NO: 2 | GCCGTGATCACCGACGAGTACAAGGTGCCCTCCAAGAAGTTCAAGGTGCTGGGC |
| Sp. Cas9 | | AACACCGACCGGCACTCCATCAAGAAGAACCTGATCGGCGCCCTGCTGTTCGAC |
| | | TCCGGCGAGACCGCCGAGGCCACCCGGCTGAAGCGGACCGCCCGGCGGCGGTAC |
| | | ACCCGGCGGAAGAACCGGATCTGCTACCTGCAGGAGATCTTCTCCAACGAGATG |
| | | GCCAAGGTGGACGACTCCTTCTTCCACCGGCTGGAGGAGTCCTTCCTGGTGGAG |
| | | GAGGACAAGAAGCACGAGCGGCACCCCATCTTCGGCAACATCGTGGACGAGGT |
| | | GGCCTACCACGAGAAGTACCCCACCATCTACCACCTGCGGAAGAAGCTGGTGGA |
| | | CTCCACCGACAAGGCCGACCTGCGGCTGATCTACCTGGCCCTGGCCCACATGAT |
| | | CAAGTTCCGGGGCCACTTCCTGATCGAGGGCGACCTGAACCCCGACAACTCCGA |
| | | CGTGGACAAGCTGTTCATCCAGCTGGTGCAGACCTACAACCAGCTGTTCGAGGA |
| | | GAACCCCATCAACGCCTCCGGCGTGGACGCCAAGGCCATCCTGTCCGCCCGGCT |
| | | GTCCAAGTCCCGGCGGCTGGAGAACCTGATCGCCCAGCTGCCCGGCGAGAAGAA |
| | | GAACGGCCTGTTCGGCAACCTGATCGCCCTGTCCCTGGGCCTGACCCCCAACTT |
| | | CAAGTCCAACTTCGACCTGGCCGAGGACGCCAAGCTGCAGCTGTCCAAGGACAC |
| | | CTACGACGACGACCTGGACAACCTGCTGGCCCAGATCGGCGACCAGTACGCCGA |
| | | CCTGTTCCTGGCCGCCAAGAACCTGTCCGACGCCATCCTGCTGTCCGACATCCT |
| | | GCGGGTGAACACCGAGATCACCAAGGCCCCCCTGTCCGCCTCCATGATCAAGCG |
| | | GTACGACGAGCACCACCAGGACCTGACCCTGCTGAAGGCCCTGGTGCGGCAGCA |
| | | GCTGCCCGAGAAGTACAAGGAGATCTTCTTCGACCAGTCCAAGAACGGCTACGC |
| | | CGGCTACATCGACGGCGGCGCCTCCCAGGAGGAGTTCTACAAGTTCATCAAGCC |
| | | CATCCTGGAGAAGATGGACGGCACCGAGGAGCTGCTGGTGAAGCTGAACCGGG |
| | | AGGACCTGCTGCGGAAGCAGCGGACCTTCGACAACGGCTCCATCCCCCACCAGA |
| | | TCCACCTGGGCGAGCTGCACGCCATCCTGCGGCGGCAGGAGGACTTCTACCCCT |
| | | TCCTGAAGGACAACCGGGAGAAGATCGAGAAGATCCTGACCTTCCGGATCCCCT |
| | | ACTACGTGGGCCCCCTGGCCCGGGGCAACTCCCGGTTCGCCTGGATGACCCGGA |
| | | AGTCCGAGGAGACCATCACCCCCTGGAACTTCGAGGAGGTGGTGGACAAGGGC |
| | | GCCTCCGCCCAGTCCTTCATCGAGCGGATGACCAACTTCGACAAGAACCTGCCC |
| | | AACGAGAAGGTGCTGCCCAAGCACTCCCTGCTGTACGAGTACTTCACCGTGTAC |
| | | AACGAGCTGACCAAGGTGAAGTACGTGACCGAGGGCATGCGGAAGCCCGCCTTC |
| | | CTGTCCGGCGAGCAGAAGAAGGCCATCGTGGACCTGCTGTTCAAGACCAACCGG |
| | | AAGGTGACCGTGAAGCAGCTGAAGGAGGACTACTTCAAGAAGATCGAGTGCTT |
| | | CGACTCCGTGGAGATCTCCGGCGTGGAGGACCGGTTCAACGCCTCCCTGGGCAC |
| | | CTACCACGACCTGCTGAAGATCATCAAGGACAAGGACTTCCTGGACAACGAGG |
| | | AGAACGAGGACATCCTGGAGGACATCGTGCTGACCCTGACCCTGTTCGAGGACC |
| | | GGGAGATGATCGAGGAGCGGCTGAAGACCTACGCCCACCTGTTCGACGACAAG |
| | | GTGATGAAGCAGCTGAAGCGGCGGCGGTACACCGGCTGGGGCCGGCTGTCCCGG |
| | | AAGCTGATCAACGGCATCCGGGACAAGCAGTCCGGCAAGACCATCCTGGACTTC |
| | | CTGAAGTCCGACGGCTTCGCCAACCGGAACTTCATGCAGCTGATCCACGACGAC |
| | | TCCCTGACCTTCAAGGAGGACATCCAGAAGGCCCAGGTGTCCGGCCAGGGCGAC |
| | | TCCCTGCACGAGCACATCGCCAACCTGGCCGGCTCCCCCGCCATCAAGAAGGGC |
| | | ATCCTGCAGACCGTGAAGGTGGTGGACGAGCTGGTGAAGGTGATGGGCCGGCA |
| | | CAAGCCCGAGAACATCGTGATCGAGATGGCCCGGGAGAACCAGACCACCCAGAA |
| | | GGGCCAGAAGAACTCCCGGGAGCGGATGAAGCGGATCGAGGAGGGCATCAAGG |
| | | AGCTGGGCTCCCAGATCCTGAAGGAGCACCCCGTGGAGAACACCCAGCTGCAGA |
| | | ACGAGAAGCTGTACCTGTACTACCTGCAGAACGGCCGGGACATGTACGTGGACC |
| | | AGGAGCTGGACATCAACCGGCTGTCCGACTACGACGTGGACCACATCGTGCCCC |
| | | AGTCCTTCCTGAAGGACGACTCCATCGACAACAAGGTGCTGACCCGGTCCGACA |
| | | AGAACCGGGGCAAGTCCGACAACGTGCCCTCCGAGGAGGTGGTGAAGAAGATG |
| | | AAGAACTACTGGCGGCAGCTGCTGAACGCCAAGCTGATCACCCAGCGGAAGTTC |
| | | GACAACCTGACCAAGGCCGAGCGGGGCGGCCTGTCCGAGCTGGACAAGGCCGGC |
| | | TTCATCAAGCGGCAGCTGGTGGAGACCCGGCAGATCACCAAGCACGTGGCCCAG |
| | | ATCCTGGACTCCCGGATGAACACCAAGTACGACGAGAACGACAAGCTGATCCGG |
| | | GAGGTGAAGGTGATCACCCTGAAGTCCAAGCTGGTGTCCGACTTCCGGAAGGA |
| | | CTTCCAGTTCTACAAGGTGCGGGAGATCAACAACTACCACCACGCCCACGACGC |
| | | CTACCTGAACGCCGTGGTGGGCACCGCCCTGATCAAGAAGTACCCCAAGCTGGA |
| | | GTCCGAGTTCGTGTACGGCGACTACAAGGTGTACGACGTGCGGAAGATGATCG |
| | | CCAAGTCCGAGCAGGAGATCGGCAAGGCCACCGCCAAGTACTTCTTCTACTCCA |
| | | ACATCATGAACTTCTTCAAGACCGAGATCACCCTGGCCAACGGCGAGATCCGGA |
| | | AGCGGCCCCTGATCGAGACCAACGGCGAGACCGGCGAGATCGTGTGGGACAAGG |
| | | GCCGGGACTTCGCCACCGTGCGGAAGGTGCTGTCCATGCCCCAGGTGAACATCG |
| | | TGAAGAAGACCGAGGTGCAGACCGGCGGCTTCTCCAAGGAGTCCATCCTGCCCA |
| | | AGCGGAACTCCGACAAGCTGATCGCCCGGAAGAAGGACTGGGACCCCAAGAAG |
| | | TACGGCGGCTTCGACTCCCCCACCGTGGCCTACTCCGTGCTGGTGGTGGCCAAG |
| | | GTGGAGAAGGGCAAGTCCAAGAAGCTGAAGTCCGTGAAGGAGCTGCTGGGCAT |
| | | CACCATCATGGAGCGGTCCTCCTTCGAGAAGAACCCCATCGACTTCCTGGAGGC |
| | | CAAGGGCTACAAGGAGGTGAAGAAGGACCTGATCATCAAGCTGCCCAAGTACT |
| | | CCCTGTTCGAGCTGGAGAACGGCCGGAAGCGGATGCTGGCCTCCGCCGGCGAGC |
| | | TGCAGAAGGGCAACGAGCTGGCCCTGCCCTCCAAGTACGTGAACTTCCTGTACC |
| | | TGGCCTCCCACTACGAGAAGCTGAAGGGCTCCCCCGAGGACAACGAGCAGAAGC |
| | | AGCTGTTCGTGGAGCAGCACAAGCACTACCTGGACGAGATCATCGAGCAGATCT |
| | | CCGAGTTCTCCAAGCGGGTGATCCTGGCCGACGCCAACCTGGACAAGGTGCTGT |
| | | CCGCCTACAACAAGCACCGGGACAAGCCCATCCGGGAGCAGGCCGAGAACATCA |
| | | TCCACCTGTTCACCCTGACCAACCTGGGCGCCCCCGCCGCCTTCAAGTACTTCGA |
| | | CACCACCATCGACCGGAAGCGGTACACCTCCACCAAGGAGGTGCTGGACGCCAC |
| | | CCTGATCCACCAGTCCATCACCGGCCTGTACGAGACCCGGATCGACCTGTCCCA |
| | | GCTGGGCGGCGACGGCGGCGGCTCCCCCAAGAAGAAGCGGAAGGTGTGA |
| open | SEQ ID | AUGGACAAGAAGUACUCCAUCGGCCUGGACAUCGGCACCAACUCCGUGGGCUG |
| reading | NO: 3 | GGCCGUGAUCACCGACGAGUACAAGGUGCCCUCCAAGAAGUUCAAGGUGCUGG |
| frame for | | GCAACACCGACCGGCACUCCAUCAAGAAGAACCUGAUCGGCGCCCUGCUGUUC |
| Cas9 with | | GACUCCGGCGAGACCGCCGAGGCCACCCGGCUGAAGCGGACCGCCCGGCGGCGG |
| Hibit tag | | UACACCCGGCGGAAGAACCGGAUCUGCUACCUGCAGGAGAUCUUCUCCAACGA |
| | | GAUGGCCAAGGUGGACGACUCCUUCUUCCACCGGCUGGAGGAGUCCUUCCUGG |
| | | UGGAGGAGGACAAGAAGCACGAGCGGCACCCCAUCUUCGGCAACAUCGUGGAC |
| | | GAGGUGGCCUACCACGAGAAGUACCCCACCAUCUACCACCUGCGGAAGAAGCU |
| | | GGUGGACUCCACCGACAAGGCCGACCUGCGGCUGAUCUACCUGGCCCUGGCCC |
| | | ACAUGAUCAAGUUCCGGGGCCACUUCCUGAUCGAGGGCGACCUGAACCCCGAC |
| | | AACUCCGACGUGGACAAGCUGUUCAUCCAGCUGGUGCAGACCUACAACCAGCU |
| | | GUUCGAGGAGAACCCCAUCAACGCCUCCGGCGUGGACGCCAAGGCCAUCCUGU |
| | | CCGCCCGGCUGUCCAAGUCCCGGCGGCUGGAGAACCUGAUCGCCCAGCUGCCCG |
| | | GCGAGAAGAAGAACGGCCUGUUCGGCAACCUGAUCGCCCUGUCCCUGGGCCUG |
| | | ACCCCCAACUUCAAGUCCAACUUCGACCUGGCCGAGGACGCCAAGCUGCAGCU |
| | | GUCCAAGGACACCUACGACGACGACCUGGACAACCUGCUGGCCCAGAUCGGCG |
| | | ACCAGUACGCCGACCUGUUCCUGGCCGCCAAGAACCUGUCCGACGCCAUCCUGC |
| | | UGUCCGACAUCCUGCGGGUGAACACCGAGAUCACCAAGGCCCCCCUGUCCGCC |
| | | UCCAUGAUCAAGCGGUACGACGAGCACCACCAGGACCUGACCCUGCUGAAGGC |
| | | CCUGGUGCGGCAGCAGCUGCCCGAGAAGUACAAGGAGAUCUUCUUCGACCAGU |
| | | CCAAGAACGGCUACGCCGGCUACAUCGACGGCGGCGCCUCCCAGGAGGAGUUC |
| | | UACAAGUUCAUCAAGCCCAUCCUGGAGAAGAUGGACGGCACCGAGGAGCUGCU |
| | | GGUGAAGCUGAACCGGGAGGACCUGCUGCGGAAGCAGCGGACCUUCGACAACG |
| | | GCUCCAUCCCCCACCAGAUCCACCUGGGCGAGCUGCACGCCAUCCUGCGGCGGC |
| | | AGGAGGACUUCUACCCCUUCCUGAAGGACAACCGGGAGAAGAUCGAGAAGAUC |
| | | CUGACCUUCCGGAUCCCCUACUACGUGGGCCCCCUGGCCCGGGGCAACUCCCGG |
| | | UUCGCCUGGAUGACCCGGAAGUCCGAGGAGACCAUCACCCCCUGGAACUUCGA |
| | | GGAGGUGGUGGACAAGGGCGCCUCCGCCCAGUCCUUCAUCGAGCGGAUGACCA |
| | | ACUUCGACAAGAACCUGCCCAACGAGAAGGUGCUGCCCAAGCACUCCCUGCUG |
| | | UACGAGUACUUCACCGUGUACAACGAGCUGACCAAGGUGAAGUACGUGACCGA |
| | | GGGCAUGCGGAAGCCCGCCUUCCUGUCCGGCGAGCAGAAGAAGGCCAUCGUGG |
| | | ACCUGCUGUUCAAGACCAACCGGAAGGUGACCGUGAAGCAGCUGAAGGAGGAC |
| | | UACUUCAAGAAGAUCGAGUGCUUCGACUCCGUGGAGAUCUCCGGCGUGGAGG |
| | | ACCGGUUCAACGCCUCCCUGGGCACCUACCACGACCUGCUGAAGAUCAUCAAG |
| | | GACAAGGACUUCCUGGACAACGAGGAGAACGAGGACAUCCUGGAGGACAUCGU |
| | | GCUGACCCUGACCCUGUUCGAGGACCGGGAGAUGAUCGAGGAGCGGCUGAAGA |
| | | CCUACGCCCACCUGUUCGACGACAAGGUGAUGAAGCAGCUGAAGCGGCGGCGG |
| | | UACACCGGCUGGGGCCGGCUGUCCCGGAAGCUGAUCAACGGCAUCCGGGACAA |
| | | GCAGUCCGGCAAGACCAUCCUGGACUUCCUGAAGUCCGACGGCUUCGCCAACC |
| | | GGAACUUCAUGCAGCUGAUCCACGACGACUCCCUGACCUUCAAGGAGGACAUC |
| | | CAGAAGGCCCAGGUGUCCGGCCAGGGCGACUCCCUGCACGAGCACAUCGCCAAC |
| | | CUGGCCGGCUCCCCCGCCAUCAAGAAGGGCAUCCUGCAGACCGUGAAGGUGGU |
| | | GGACGAGCUGGUGAAGGUGAUGGGCCGGCACAAGCCCGAGAACAUCGUGAUCG |
| | | AGAUGGCCCGGGAGAACCAGACCACCCAGAAGGGCCAGAAGAACUCCCGGGAG |
| | | CGGAUGAAGCGGAUCGAGGAGGGCAUCAAGGAGCUGGGCUCCCAGAUCCUGAA |
| | | GGAGCACCCCGUGGAGAACACCCAGCUGCAGAACGAGAAGCUGUACCUGUACU |
| | | ACCUGCAGAACGGCCGGGACAUGUACGUGGACCAGGAGCUGGACAUCAACCGG |
| | | CUGUCCGACUACGACGUGGACCACAUCGUGCCCCAGUCCUUCCUGAAGGACGA |
| | | CUCCAUCGACAACAAGGUGCUGACCCGGUCCGACAAGAACCGGGGCAAGUCCG |
| | | ACAACGUGCCCUCCGAGGAGGUGGUGAAGAAGAUGAAGAACUACUGGCGGCAG |
| | | CUGCUGAACGCCAAGCUGAUCACCCAGCGGAAGUUCGACAACCUGACCAAGGC |
| | | CGAGCGGGGCGGCCUGUCCGAGCUGGACAAGGCCGGCUUCAUCAAGCGGCAGC |
| | | UGGUGGAGACCCGGCAGAUCACCAAGCACGUGGCCCAGAUCCUGGACUCCCGG |
| | | AUGAACACCAAGUACGACGAGAACGACAAGCUGAUCCGGGAGGUGAAGGUGA |
| | | UCACCCUGAAGUCCAAGCUGGUGUCCGACUUCCGGAAGGACUUCCAGUUCUAC |
| | | AAGGUGCGGGAGAUCAACAACUACCACCACGCCCACGACGCCUACCUGAACGC |
| | | CGUGGUGGGCACCGCCCUGAUCAAGAAGUACCCCAAGCUGGAGUCCGAGUUCG |
| | | UGUACGGCGACUACAAGGUGUACGACGUGCGGAAGAUGAUCGCCAAGUCCGAG |
| | | CAGGAGAUCGGCAAGGCCACCGCCAAGUACUUCUUCUACUCCAACAUCAUGAA |
| | | CUUCUUCAAGACCGAGAUCACCCUGGCCAACGGCGAGAUCCGGAAGCGGCCCC |
| | | UGAUCGAGACCAACGGCGAGACCGGCGAGAUCGUGUGGGACAAGGGCCGGGAC |
| | | UUCGCCACCGUGCGGAAGGUGCUGUCCAUGCCCCAGGUGAACAUCGUGAAGAA |
| | | GACCGAGGUGCAGACCGGCGGCUUCUCCAAGGAGUCCAUCCUGCCCAAGCGGA |
| | | ACUCCGACAAGCUGAUCGCCCGGAAGAAGGACUGGGACCCCAAGAAGUACGGC |
| | | GGCUUCGACUCCCCCACCGUGGCCUACUCCGUGCUGGUGGUGGCCAAGGUGGA |
| | | GAAGGGCAAGUCCAAGAAGCUGAAGUCCGUGAAGGAGCUGCUGGGCAUCACCA |
| | | UCAUGGAGCGGUCCUCCUUCGAGAAGAACCCCAUCGACUUCCUGGAGGCCAAG |
| | | GGCUACAAGGAGGUGAAGAAGGACCUGAUCAUCAAGCUGCCCAAGUACUCCCU |
| | | GUUCGAGCUGGAGAACGGCCGGAAGCGGAUGCUGGCCUCCGCCGGCGAGCUGC |
| | | AGAAGGGCAACGAGCUGGCCCUGCCCUCCAAGUACGUGAACUUCCUGUACCUG |
| | | GCCUCCCACUACGAGAAGCUGAAGGGCUCCCCCGAGGACAACGAGCAGAAGCA |
| | | GCUGUUCGUGGAGCAGCACAAGCACUACCUGGACGAGAUCAUCGAGCAGAUCU |
| | | CCGAGUUCUCCAAGCGGGUGAUCCUGGCCGACGCCAACCUGGACAAGGUGCUG |
| | | UCCGCCUACAACAAGCACCGGGACAAGCCCAUCCGGGAGCAGGCCGAGAACAU |
| | | CAUCCACCUGUUCACCCUGACCAACCUGGGCGCCCCCGCCGCCUUCAAGUACUU |
| | | CGACACCACCAUCGACCGGAAGCGGUACACCUCCACCAAGGAGGUGCUGGACG |
| | | CCACCCUGAUCCACCAGUCCAUCACCGGCCUGUACGAGACCCGGAUCGACCUG |
| | | UCCCAGCUGGGCGGCGACGGCGGCGGCUCCCCCAAGAAGAAGCGGAAGGUGUC |
| | | CGAGUCCGCCACCCCCGAGUCCGUGUCCGGCUGGCGGCUGUUCAAGAAGAUCU |
| | | CCUGA |
| amino acid | SEQ ID | MDKKYSIGLDIGTNSVGWAVITDEYKVPSKKFKVLGNTDRHSIKKNLIGAL |
| sequence | NO: 4 | LFDSGETAEATRLKRTARRRYTRRKNRICYLQEIFSNEMAKVDDSFFHRLEESFL |
| encoded by | | VEEDKKHERHPIFGNIVDEVAYHEKYPTIYHLRKKLVDSTDKADLRLIYLALAH |
| SEQ ID | | MIKFRGHFLIEGDLNPDNSDVDKLFIQLVQTYNQLFEENPINASGVDAKAILSAR |
| NOs: 1-3 of | | LSKSRRLENLIAQLPGEKKNGLFGNLIALSLGLTPNFKSNFDLAEDAKLQLSKDT |
| Cas9 | | YDDDLDNLLAQIGDQYADLFLAAKNLSDAILLSDILRVNTEITKAPLSASMIKRY |
| | | DEHHQDLTLLKALVRQQLPEKYKEIFFDQSKNGYAGYIDGGASQEEFYKFIKPIL |
| | | EKMDGTEELLVKLNREDLLRKQRTFDNGSIPHQIHLGELHAILRRQEDFYPFLK |
| | | DNREKIEKILTFRIPYYVGPLARGNSRFAWMTRKSEETITPWNFEEVVDKGASA |
| | | QSFIERMTNFDKNLPNEKVLPKHSLLYEYFTVYNELTKVKYVTEGMRKPAFLSG |
| | | EQKKAIVDLLFKTNRKVTVKQLKEDYFKKIECFDSVEISGVEDRFNASLGTYHDL |
| | | LKIIKDKDFLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHDDSLTFKE |
| | | DIQKAQVSGQGDSLHEHIANLAGSPAIKKGILQTVKVVDELVKVMGRHKPENIVI |
| | | EMARENQTTQKGQKNSRERMKRIEEGIKELGSQILKEHPVENTQLQNEKLYLYY |
| | | LQNGRDMYVDQELDINRLSDYDVDHIVPQSFLKDDSIDNKVLTRSDKNRGKSD |
| | | NVPSEEVVKKMKNYWRQLLNAKLITQRKFDNLTKAERGGLSELDKAGFIKRQL |
| | | VETRQITKHVAQILDSRMNTKYDENDKLIREVKVITLKSKLVSDFRKDFQFYKVR |
| | | EINNYHHAHDAYLNAVVGTALIKKYPKLESEFVYGDYKVYDVRKMIAKSEQEIG |
| | | KATAKYFFYSNIMNFFKTEITLANGEIRKRPLIETNGETGEIVWDKGRDFATVRK |
| | | VLSMPQVNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYKEVKKDLI |
| | | IKLPKYSLFELENGRKRMLASAGELQKGNELALPSKYVNFLYLASHYEKLKGSPE |
| | | DNEQKQLFVEQHKHYLDEIIEQISEFSKRVILADANLDKVLSAYNKHRDKPIREQ |
| | | AENIIHLFTLTNLGAPAAFKYFDTTIDRKRYTSTKEVLDATLIHQSITGLYETRID |
| | | LSQLGGDGGGSPKKKRKV* |
| amino acid | SEQ ID | MDKKYSIGLDIGTNSVGWAVITDEYKVPSKKFKVLGNTDRHSIKKNLIG |
| sequence for | NO: 5 | ALLFDSGETAEATRLKRTARRRYTRRKNRICYLQEIFSNEMAKVDDSFFH |
| Sp Cas9- | | RLEESFLVEEDKKHERHPIFGNIVDEVAYHEKYPTIYHLRKKLVDSTDKADLRLI |
| Hibit fusion | | YLALAHMIKFRGHFLIEGDLNPDNSDVDKLFIQLVQTYNQLFEENPINASGVDAK |
| | | AILSARLSKSRRLENLIAQLPGEKKNGLFGNLIALSLGLTPNFKSNFDLAEDAKLQ |
| | | LSKDTYDDDLDNLLAQIGDQYADLFLAAKNLSDAILLSDILRVNTEITKAPLSAS |
| | | MIKRYDEHHQDLTLLKALVRQQLPEKYKEIFFDQSKNGYAGYIDGGASQEEFYK |
| | | FIKPILEKMDGTEELLVKLNREDLLRKQRTFDNGSIPHQIHLGELHAILRRQEDF |
| | | YPFLKDNREKIEKILTFRIPYYVGPLARGNSRFAWMTRKSEETITPWNFEEVVD |
| | | KGASAQSFIERMTNFDKNLPNEKVLPKHSLLYEYFTVYNELTKVKYVTEGMRKP |
| | | AFLSGEQKKAIVDLLFKTNRKVTVKQLKEDYFKKIECFDSVEISGVEDRFNASLG |
| | | TYHDLLKIIKDKDFLDNEENEDILEDIVLTLTLFEDREMIEERLKTYAHLFDDKV |
| | | MKQLKRRRYTGWGRLSRKLINGIRDKQSGKTILDFLKSDGFANRNFMQLIHDDS |
| | | LTFKEDIQKAQVSGQGDSLHEHIANLAGSPAIKKGILQTVKVVDELVKVMGRHK |
| | | PENIVIEMARENQTTQKGQKNSRERMKRIEEGIKELGSQILKEHPVENTQLQNE |
| | | KLYLYYLQNGRDMYVDQELDINRLSDYDVDHIVPQSFLKDDSIDNKVLTRSDKN |
| | | RGKSDNVPSEEVVKKMKNYWRQLLNAKLITQRKFDNLTKAERGGLSELDKAGF |
| | | IKRQLVETRQITKHVAQILDSRMNTKYDENDKLIREVKVITLKSKLVSDFRKDFQ |
| | | FYKVREINNYHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVWDKGRDF |
| | | ATVRKVLSMPQVNIVKKTEVQTGGFSKESILPKRNSDKLIARKKDWDPKKYGGF |
| | | DSPTVAYSVLVVAKVEKGKSKKLKSVKELLGITIMERSSFEKNPIDFLEAKGYKEV |
| | | KKDLIIKLPKYSLFELENGRKRMLASAGELQKGNELALPSKYVNFLYLASHYEKL |
| | | KGSPEDNEQKQLFVEQHKHYLDEIIEQISEFSKRVILADANLDKVLSAYNKHRDK |
| | | PIREQAENIIHLFTLTNLGAPAAFKYFDTTIDRKRYTSTKEVLDATLIHQSITGLY |
| | | ETRIDLSQLGGDGGGSPKKKRKVSESATPESVSGWRLFKKIS |
| Full HDRT | SEQ ID | GAGGGCCGCGGCAGCCTGCTGACCTGCGGCGACGTGGAGGAGAAtCCCGGCCCC |
| template - | NO: 6 | ATGgtgAGCAAGGGCGAGGAGCTGTTCACCGGGGTGGTGCCCATCCTGGTCGAG |
| GFP T2A | | CTGGACGGCGACGTAAACGGCCACAAGTTCAGCGTGTCCGGCGAGGGCGAGGGC |
| insert | | GATGCCACCTACGGCAAGCTGACCCTGAAGTTCATCTGCACCACCGGCAAGCTG |
| GFP: | | CCCGTGCCCTGGCCCACCCTCGTGACCACCCTGACCTACGGCGTGCAGTGCTTCA |
| P00894 | | GCCGCTACCCCGACCACATGAAGCAGCACGACTTCTTCAAGTCCGCCATGCCCG |
| | | AAGGCTACGTCCAGGAGCGCACCATCTTCTTCAAGGACGACGGCAACTACAAGA |
| | | CCCGCGCCGAGGTGAAGTTCGAGGGCGACACCCTGGTGAACCGCATCGAGCTGA |
| | | AGGGCATCGACTTCAAGGAGGACGGCAACATCCTGGGGCACAAGCTGGAGTAC |
| | | AACTACAACAGCCACAACGTCTATATCATGGCCGACAAGCAGAAGAACGGCAT |
| | | CAAGGTGAACTTCAAGATCCGCCACAACATCGAGGACGGCAGCGTGCAGCTCGC |
| | | CGACCACTACCAGCAGAACACCCCCATCGGCGACGGCCCCGTGCTGCTGCCCGA |
| | | CAACCACTACCTGAGCACCCAGTCCGCCCTGAGCAAAGACCCCAACGAGAAGCG |
| | | CGATCACATGGTCCTGCTGGAGTTCGTGACCGCCGCCGGGATCACTCTCGGCAT |
| | | GGACGAGCTGTACAAGTAAcctCGACTGTGCCTTCTAGTTGCCAGCCATCTGTTG |
| | | TTTGCCCCTCCCCCGTGCCTTCCTTGACCCTGGAAGGTGCCACTCCCACTGTCCT |
| | | TTCCTAATAAAATGAGGAAATTGCATCGCATTGTCTGAGTAGGTGTCATTCTA |
| | | TTCTGGGGGGTGGGGTGGGGCAGGACAGCAAGGGGGAGGATTGGGAAGACAAT |
| | | AGCAGGCATGCTGGGGATGCGGTGGGCTCTATGGcttctgaggcggaaagaaccag |
| | | ctggggctctagggggtatccccACTAGTCGTGTACCAGCTGAGAGACTCTAAATC |
| | | CAGTGACAAGTCTGTCTGCCTATTCACCGATTTTGATTCTCAAACAAATGTGTCAC |
| | | AAAGTAAGGATTCTGATGTGTATATCACAGACAAAACTGTGCTAGACATGAGGTCT |
| | | ATGGACTTCAAGAGCAACAGTGCTGTGGCCTGGAGCAACAAATCTGACTTTGCA |
| | | TGTGCAAACGCCTTCAACAACAGCATTATTCCAGAAGACACCTTCTTCCCCAGC |
| | | CCAGgtaagggcagctttggtgccttcgcaggctgtttccttgcttcaggaatggc |
| | | caggttctgcccagagctctggtcaatgatgtctaaaactcctctgattggtggtc |
| | | tcggccttatccattgccaccaaaaccctctttttactaagaaacagtgagccttg |
| | | ttctggcagtccagagaatgacacgggaaaaaagcagatgaagagaaggtggcagg |
| | | agagggcacgtggcccagcctcagtctct |
| GFP insert | SEQ ID | ATGGTGAGCAAGGGCGAGGAGCTGTTCACCGGGGTGGTGCCCATCC |
| for HDRT - | NO: 7 | TGGTCGAGCTGGACGGCGACGTAAACGGCCACAAGTTCAGCGTGTCCGGC |
| GFP: | | GAGGGCGAGGGCGATGCCACCTACGGCAAGCTGACCCTGAAGTTCATCTGCACC |
| P00894 | | ACCGGCAAGCTGCCCGTGCCCTGGCCCACCCTCGTGACCACCCTGACCTACGGCG |
| | | TGCAGTGCTTCAGCCGCTACCCCGACCACATGAAGCAGCACGACTTCTTCAAGT |
| | | CCGCCATGCCCGAAGGCTACGTCCAGGAGCGCACCATCTTCTTCAAGGACGACG |
| | | GCAACTACAAGACCCGCGCCGAGGTGAAGTTCGAGGGCGACACCCTGGTGAACC |
| | | GCATCGAGCTGAAGGGCATCGACTTCAAGGAGGACGGCAACATCCTGGGGCAC |
| | | AAGCTGGAGTACAACTACAACAGCCACAACGTCTATATCATGGCCGACAAGCA |
| | | GAAGAACGGCATCAAGGTGAACTTCAAGATCCGCCACAACATCGAGGACGGCA |
| | | GCGTGCAGCTCGCCGACCACTACCAGCAGAACACCCCCATCGGCGACGGCCCCG |
| | | TGCTGCTGCCCGACAACCACTACCTGAGCACCCAGTCCGCCCTGAGCAAAGACC |
| | | CCAACGAGAAGCGCGATCACATGGTCCTGCTGGAGTTCGTGACCGCCGCCGGGA |
| | | TCACTCTCGGCATGGACGAGCTGTACAAGTAA |
| TCR α chain | SEQ ID | METLLKVLSGTLLWQLTWVRSQQPVQSPQAVILREGEDAVINCSSSKAL |
| pINT1066 | NO: 8 | YSVHWYRQKHGEAPVFLMILLKGGEQKGHEKISASFNEKKQQSSLYLTAS |
| | | QLSYSGTYFCGTAWINDYKLSFGAGTTVTVRANIQNPDPAVYQLRDSKSSDKSV |
| | | CLFTDFDSQTNVSQSKDSDVYITDKTVLDMRSMDFKSNSAVAWSNKSDFACAN |
| | | AFNNSIIPEDTFFPSPESSCDVKLVEKSFETDTNLNFQNLSVIGFRILLLKVAGFNL |
| | | LMTLRLWSS* |
| eGFP ORF | SEQ ID | ATGGTGAGCAAGGGCGAGGAGCTGTTCACCGGGGTGGTGCCCATCCTGGTCG |
| GFP: | NO: 9 | AGCTGGACGGCGACGTAAACGGCCACAAGTTCAGCGTGTCCGGCGAGGGCGAG |
| P00894, | | GGCGATGCCACCTACGGCAAGCTGACCCTGAAGTTCATCTGCACCACCGGCAAG |
| GFP | | CTGCCCGTGCCCTGGCCCACCCTCGTGACCACCCTGACCTACGGCGTGCAGTGCT |
| P01018 | | TCAGCCGCTACCCCGACCACATGAAGCAGCACGACTTCTTCAAGTCCGCCATGC |
| | | CCGAAGGCTACGTCCAGGAGCGCACCATCTTCTTCAAGGACGACGGCAACTACA |
| | | AGACCCGCGCCGAGGTGAAGTTCGAGGGCGACACCCTGGTGAACCGCATCGAGC |
| | | TGAAGGGCATCGACTTCAAGGAGGACGGCAACATCCTGGGGCACAAGCTGGAG |
| | | TACAACTACAACAGCCACAACGTCTATATCATGGCCGACAAGCAGAAGAACGG |
| | | CATCAAGGTGAACTTCAAGATCCGCCACAACATCGAGGACGGCAGCGTGCAGCT |
| | | CGCCGACCACTACCAGCAGAACACCCCCATCGGCGACGGCCCCGTGCTGCTGCCC |
| | | GACAACCACTACCTGAGCACCCAGTCCGCCCTGAGCAAAGACCCCAACGAGAAG |
| | | CGCGATCACATGGTCCTGCTGGAGTTCGTGACCGCCGCCGGGATCACTCTCGGC |
| | | ATGGACGAGCTGTACAAGTAA |
| gRNA | SEQ ID | mC*mU*mC*UCAGCUGGUACACGGCAGUUUUAGAmGmCmUmAmGm |
| targeting | NO: 10 | AmAmAmUmAmGmCAAGUUAAAAUAAGGCUAGUCCGUUAUCAmAm |
| TRAC | | CmUmUmGmAmAmAmAmAmGmUmGmGmCmAmCmCmGmAmGmUmC |
| | | mGmGmUmGmCmU*mU*mU*mU |
| sgRNA | SEQ ID | mC*mC*mA*AUAUCAGGAGACUAGGAGUUUUAGAmGmCmUmAmGmAmAm |
| targeting | NO: 11 | AmUmAmGmCAAGUUAAAAUAAGGCUAGUCCGUUAUCAmAmCmUmUmGm |
| AAVS1 | | AmAmAmAmAmGmUmGmGmCmAmCmCmGmAmGmUmCmGmGmUmGmC |
| | | mU*mU*mU*mU |
| 41BBL | 12 | MEYASDASLDPEAPWPPAPRARACRVLPWALVAGLLLLLLLAAAC |
| | | AVFLACPWAVSGARASPGSAASPRLREGPELSPDDPAGLLDLRQGM |
| | | FAQLVAQNVLLIDGPLSWYSDPGLAGVSLTGGLSYKEDTKELVVAK |
| | | AGVYYVFFQLELRRVVAGEGSGSVSLALHLQPLRSAAGAAALALTV |
| | | DLPPASSEARNSAFGFQGRLLHLSAGQRLGVHLHTEARARHAWQLT |
| | | QGATVLGLFRVTPEIPAGLPSPRSE |
| mIL21 | 13 | MDWTWILFLVAAATRVHSHKSSSQGQDRHMIRMRQLIDIVDQLKN |
| | | YVNDLVPEFLPAPEDVETNCEWSAFSCFQKAQLKSANTGNNERIINV |
| | | SIKKLKRKPPSTNAGRRQKHRLTCPSCDSYEKKPPKEFLERFKSLLQK |
| | | MIHQHLSSRTHGSEDSEQKLISEEDLTTTPAPRPPTPAPTIASQPLSLRP |
| | | EACRPAAGGAVHTRGLDFACDFWVLVVVGGVLACYSLLVTVAFIIF |
| | | WV |

What is claimed is:

1. A lipid composition comprising:
a biologically active agent; and
a lipid component, wherein the lipid component comprises:
a) an ionizable lipid in an amount from about 25-50 mol % of the lipid component;
b) a neutral lipid in an amount from about 7-25 mol % of the lipid component;
c) a helper lipid in an amount from about 39-65 mol % of the lipid component; and
d) a PEG lipid in an amount from about 0.5-1.8 mol % of the lipid component;
wherein the ionizable lipid is a compound of Formula (I) 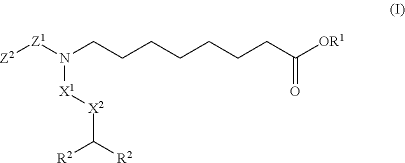
wherein
X¹ is C₆₋₇ alkylene;
X² is 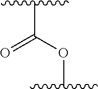 [SMILES: *OC(*)=O]
or absent, provided that if X² is 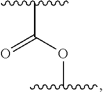 [SMILES: *OC(*)=O]
R² is not alkoxy;
Z¹ is C₂₋₃ alkylene;
Z² is selected from —OH, —NHC(═O)OCH₃, and —NHS(═O)₂CH₃;
R¹ is C₇₋₉ unbranched alkyl or C₇₋₁₁ unbranched alkynyl; and
each R² is independently C₈ alkyl or C₈ alkoxy;
or a salt thereof.

2. A lipid composition comprising:
a biologically active agent; and
a lipid component, wherein the lipid component comprises:
a) an ionizable lipid in an amount from about 25-50 mol % of the lipid component;
b) a neutral lipid in an amount from about 7-25 mol % of the lipid component;
c) a helper lipid in an amount from about 39-65 mol % of the lipid component; and
d) a PEG lipid in an amount from about 0.5-1.8 mol % of the lipid component;
wherein the ionizable lipid is a compound of Formula (I) 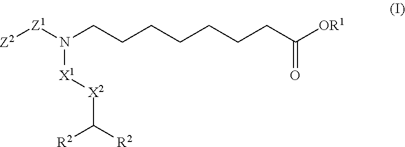
wherein
X¹ is C₆₋₇ alkylene;
X² is 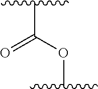 [SMILES: *OC(*)=O]
or absent, provided that if X² is 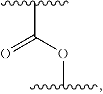 [SMILES: *OC(*)=O]
R² is not alkoxy;
Z¹ is C₂₋₃ alkylene;
Z² is selected from —OH, —NHC(═O)OCH₃, and —NHS(═O)₂CH₃;
R¹ is C₇₋₉ unbranched alkyl; and
each R² is independently C₈ alkyl or C₈ alkoxy;
or a salt thereof.

3. The lipid composition of claim 1 or 2, wherein the ionizable lipid is a compound of Formula (II) 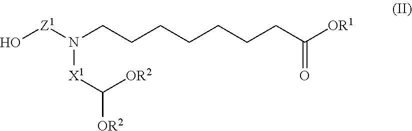
wherein
X¹ is C₆₋₇ alkylene;
Z¹ is C₂₋₃ alkylene;
R¹ is C₇₋₉ unbranched alkyl; and
each R² is C₈ alkyl;
or a salt thereof.

4. A lipid composition comprising:
a biologically active agent; and
a lipid component, wherein the lipid component comprises:
a) an ionizable lipid in an amount from about 25-50 mol % of the lipid component;
b) a neutral lipid in an amount from about 7-25 mol % of the lipid component;
c) a helper lipid in an amount from about 39-65 mol % of the lipid component; and
d) a PEG lipid in an amount from about 0.5-1.8 mol % of the lipid component;
wherein the ionizable lipid is 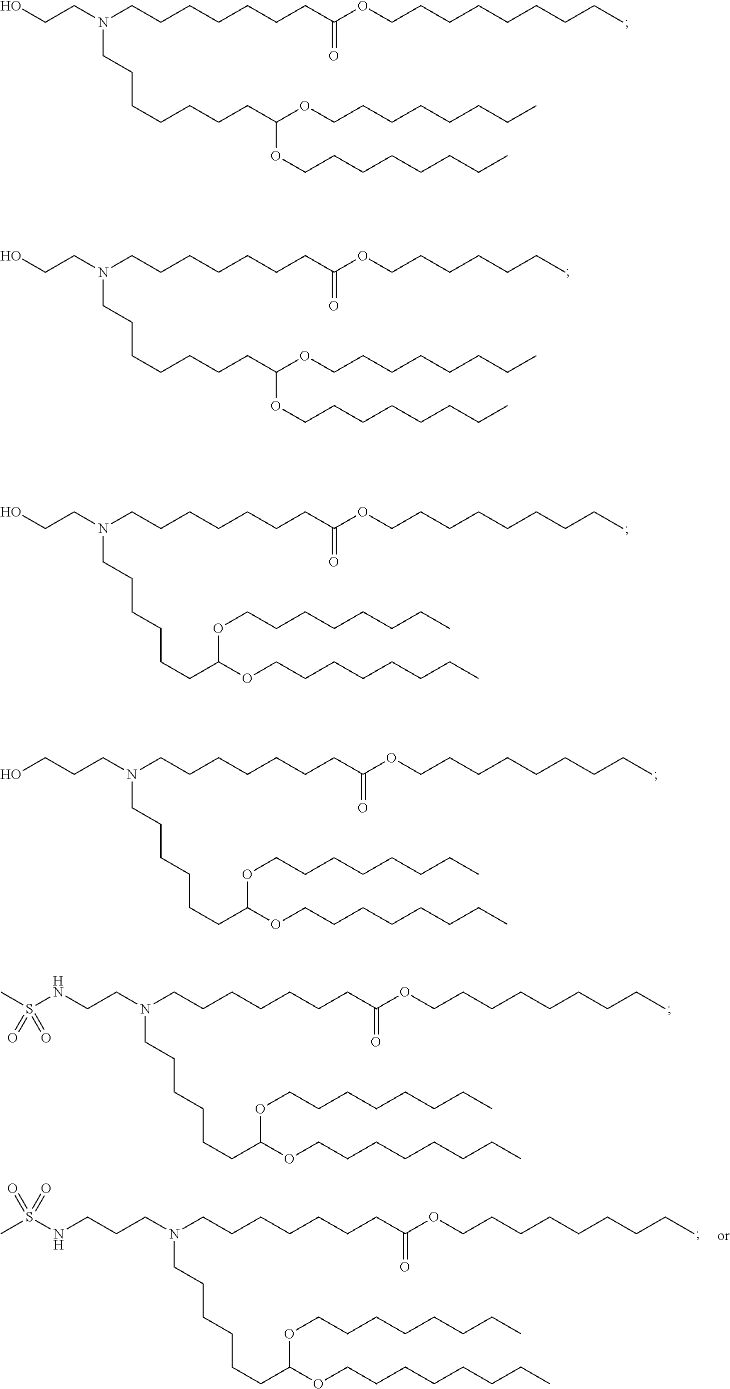 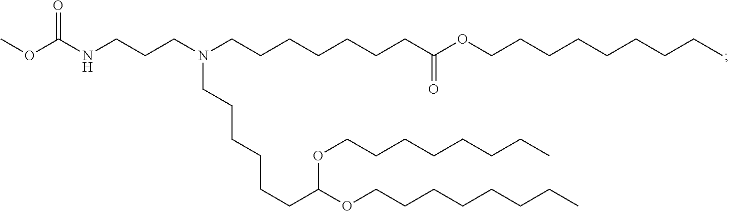 [SMILES: CCCCCCCCCOC(=O)CCCCCCCN(CCCCCCC(OCCCCCCCC)OCCCCCCCC)CCCNC(=O)OC]
or a salt thereof.

5. The lipid composition of any one of the preceding claims, wherein the ionizable lipid is 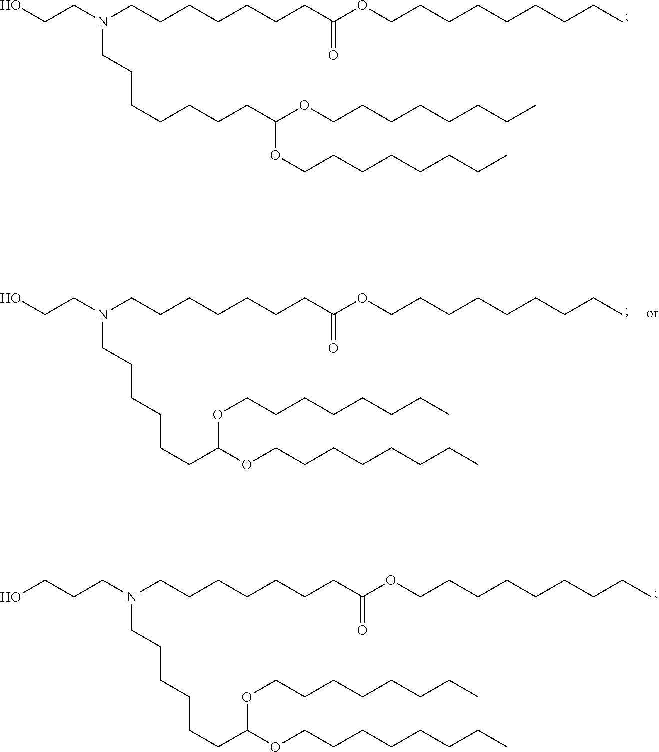 [SMILES: CCCCCCCCCOC(=O)CCCCCCCN(CCO)CCCCCCCC(OCCCCCCCC)OCCCCCCCC ; CCCCCCCCCOC(=O)CCCCCCCN(CCO)CCCCCCC(OCCCCCCCC)OCCCCCCCC ; CCCCCCCCCOC(=O)CCCCCCCN(CCCO)CCCCCCC(OCCCCCCCC)OCCCCCCCC]
or a salt thereof.

6. The lipid composition of any one of the preceding claims, wherein the ionizable lipid is 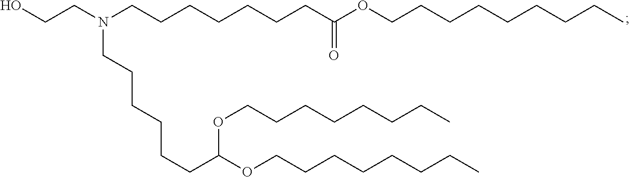 [SMILES: CCCCCCCCCOC(=O)CCCCCCCN(CCO)CCCCCCC(OCCCCCCCC)OCCCCCCCC]
or a salt thereof.

7. The lipid composition of any one of the preceding claims, wherein the neutral lipid is an uncharged lipid or a zwitterionic lipid.

8. The lipid composition of any one of the preceding claims, wherein the neutral lipid is DSPC or DPME.

9. The lipid composition of any one of the preceding claims, wherein the neutral lipid is DSPC.

10. The lipid composition of any one of the preceding claims, wherein the helper lipid is selected from cholesterol, 5-heptadecylresorcinol, and cholesterol hemisuccinate.

11. The lipid composition of any one of the preceding claims, wherein the helper lipid is cholesterol.

12. The lipid composition of any one of the preceding claims, wherein the PEG lipid comprises dimyristoylglycerol (DMG).

13. The lipid composition of any one of the preceding claims, wherein the PEG lipid comprises PEG-2k.

14. The lipid composition of any one of the preceding claims, wherein the PEG lipid is a PEG-DMG.

15. The lipid composition of any one of the preceding claims, wherein the PEG lipid is PEG-2k DMG.

16. The lipid composition of any one of the preceding claims, wherein the ionizable lipid is 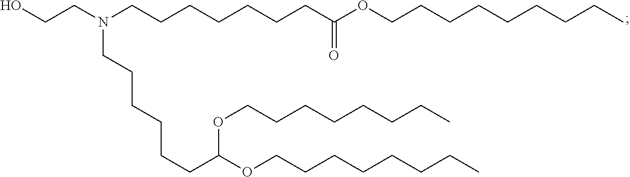 [SMILES: CCCCCCCCCOC(=O)CCCCCCCN(CCO)CCCCCCC(OCCCCCCCC)OCCCCCCCC]
the neutral lipid is DSPC; the helper lipid is cholesterol; and the PEG lipid is 1,2-dimyristoyl-rac-glycero-3-methoxypolyethylene glycol-2000.

17. The lipid composition of any one of the preceding claims, wherein the amount of the ionizable lipid is from about 30-45 mol % of the lipid component.

18. The lipid composition of any one of claims 1-16, wherein the amount of the ionizable lipid is from about 30-40 mol % of the lipid component.

19. The lipid composition of any one of claims 1-16, wherein the amount of the ionizable lipid is about 30 mol % of the lipid component.

20. The lipid composition of any one of claims 1-16, wherein the amount of the ionizable lipid is about 40 mol % of the lipid component.

21. The lipid composition of any one of claims 1-16, wherein the amount of the ionizable lipid is about 50 mol % of the lipid component.

22. The lipid composition of any one of the preceding claims, wherein the amount of the neutral lipid is from about 10-20 mol % of the lipid component.

23. The lipid composition of any one of claims 1-21, wherein the amount of the neutral lipid is from about 10-15 mol % of the lipid component.

24. The lipid composition of any one of claims 1-21, wherein the amount of the neutral lipid is about 10 mol % of the lipid component.

25. The lipid composition of any one of claims 1-21, wherein the amount of the neutral lipid is about 15 mol % of the lipid component.

26. The lipid composition of any one of the preceding claims, wherein the amount of the helper lipid is from about 50-60 mol % of the lipid component.

27. The lipid composition of any one of claims 1-25, wherein the amount of the helper lipid is from about 39-59 mol % of the lipid component.

28. The lipid composition of any one of claims 1-25, wherein the amount of the helper lipid is from about 43.5-59 mol % of the lipid component.

29. The lipid composition of any one of claims 1-25, wherein the amount of the helper lipid is about 59 mol % of the lipid component.

30. The lipid composition of any one of claims 1-25, wherein the amount of the helper lipid is about 43.5 mol % of the lipid component.

31. The lipid composition of any one of claims 1-25, wherein the amount of the helper lipid is about 39 mol % of the lipid component.

32. The lipid composition of any one of the preceding claims, wherein the amount of the PEG lipid is from about 0.9-1.6 mol % of the lipid component.

33. The lipid composition of any one of claims 1-31, wherein the amount of the PEG lipid is from about 1-1.5 mol % of the lipid component.

34. The lipid composition of any one of claims 1-31, wherein the amount of the PEG lipid is about 1 mol % of the lipid component.

35. The lipid composition of any one of claims 1-31, wherein the amount of the PEG lipid is about 1.5 mol % of the lipid component.

36. The lipid composition of any one of claims 1-16, wherein the amount of the ionizable lipid is from about 27-40 mol % of the lipid component; the amount of the neutral lipid is from about 10-20 mol % of the lipid component; the amount of the helper lipid is from about 50-60 mol % of the lipid component; and the amount of the PEG lipid is from about 0.9-1.6 mol % of the lipid component.

37. The lipid composition of any one of claims 1-16, wherein the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component.

38. The lipid composition of any one of claims 1-16, wherein the amount of the ionizable lipid is about 30 mol % of the lipid component; the amount of the neutral lipid is about 10 mol % of the lipid component; the amount of the helper lipid is about 59 mol % of the lipid component; and the amount of the PEG lipid is about 1-1.5 mol % of the lipid component.

39. The lipid composition of any one of claims 1-16, wherein the amount of the ionizable lipid is about 40 mol % of the lipid component; the amount of the neutral lipid is about 15 mol % of the lipid component; the amount of the helper lipid is about 43.5 mol % of the lipid component; and the amount of the PEG lipid is about 1.5 mol % of the lipid component.

40. The lipid composition of any one of claims 1-16, wherein the amount of the ionizable lipid is about 50 mol % of the lipid component; the amount of the neutral lipid is about 10 mol % of the lipid component; the amount of the helper lipid is about 39 mol % of the lipid component; and the amount of the PEG lipid is about 1 mol % of the lipid component.

41. The lipid composition of any one of the preceding claims, wherein each mol % varies by less than 5%.

42. The lipid composition of any one of the preceding claims, wherein each mol % varies by less than 1%.

43. The lipid composition of any one of the preceding claims, wherein each mol % varies by less than 0.5%.

44. The lipid composition of any one of the preceding claims, wherein each mol % is based on the relative nominal concentrations of the ionizable lipid, the neutral lipid, the helper lipid, and the PEG lipid.

45. The lipid composition of any one of the preceding claims, wherein each mol % is based on the relative actual concentrations of the ionizable lipid, the neutral lipid, the helper lipid, and the PEG lipid.

46. The lipid composition of any one of the preceding claims, wherein the lipid composition is in the form of LNPs; and the LNPs have a Z-average diameter of less than about 100 nm.

47. The lipid composition of any one of the preceding claims, wherein the lipid composition is in the form of LNPs; and the LNPs have a Z-average diameter of less than about 95 nm.

48. The lipid composition of any one of the preceding claims, wherein the lipid composition is in the form of LNPs; and the LNPs have a Z-average diameter of less than about 90 nm.

49. The lipid composition of any one of the preceding claims, wherein the lipid composition is in the form of LNPs; and the LNPs have number-average diameter of greater than about 45 nm.

50. The lipid composition of any one of the preceding claims, wherein the lipid composition is in the form of LNPs; and the LNPs have number-average diameter of greater than about 50 nm.

51. The lipid composition of any one of the preceding claims, wherein the lipid composition is in the form of LNPs; and the LNPs have a polydispersity index of about 0.005 to about 0.75.

52. The lipid composition of any one of the preceding claims, wherein the LNP composition is in the form of LNPs; and the LNPs have a polydispersity index of about 0.005 to about 0.1.

53. The lipid composition of any one of the preceding claims, wherein the N/P ratio of the lipid composition is from about 5 to about 7.

54. The lipid composition of any one of the preceding claims, wherein the N/P ratio of the lipid composition is about 6.

55. The lipid composition of any one of the preceding claims, wherein the biologically active agent comprises a non-nucleic acid component.

56. The lipid composition of any one of the preceding claims, wherein the biologically active agent comprises or encodes a therapeutically active protein.

57. The lipid composition of any one of the preceding claims, wherein the biologically active agent comprises or encodes a genome-editing tool.

58. The lipid composition of any one of the preceding claims, wherein the biologically active agent comprises or encodes one or more nucleases capable of making single or double strand break in a DNA or an RNA.

59. The lipid composition of any one of the preceding claims, wherein the biologically active agent comprises a nucleic acid component.

60. The lipid composition of any one of the preceding claims, wherein the biologically active agent comprises an RNA.

61. The lipid composition of claim 60, wherein the RNA is an mRNA.

62. The lipid composition of claim 61, wherein the nucleic acid component comprises an mRNA encoding an RNA-guided DNA-binding agent.

63. The lipid composition of claim 62, wherein the mRNA comprises a Cas nuclease mRNA.

64. The lipid composition of claim 62, wherein the mRNA comprises a Class 2 Cas nuclease mRNA.

65. The lipid composition of claim 62, wherein the mRNA comprises a Cas9 nuclease mRNA.

66. The lipid composition of any one of claims 59-65, wherein the nucleic acid component comprises a modified RNA.

67. The lipid composition of any one of claims 59-66, wherein the nucleic acid component comprises a guide RNA nucleic acid.

68. The lipid composition of claim 67, wherein the guide RNA nucleic acid is a gRNA.

69. The lipid composition of claim 67 or 68, wherein the guide RNA nucleic acid is or encodes a dual-guide RNA (dgRNA).

70. The lipid composition of claim 67 or 68, wherein the guide RNA nucleic acid is or encodes a single-guide (sgRNA).

71. The lipid composition of any one of claims 68-70, wherein the gRNA is a modified gRNA.

72. The lipid composition of claim 71, wherein the modified gRNA comprises a modification at one or more of the first five nucleotides at a 5′ end.

73. The lipid composition of claim 71 or 72, wherein the modified gRNA comprises a modification at one or more of the last five nucleotides at a 3′ end.

74. The lipid composition of any one of claims 59-73, wherein the nucleic acid component comprises a guide RNA nucleic acid; the mRNA is a Class 2 Cas nuclease mRNA; and the ratio of the mRNA to the guide RNA nucleic acid is from about 2:1 to 1:4 by weight.

75. The lipid composition of claim 74, wherein the ratio of the guide RNA nucleic acid to the Class 2 Cas nuclease mRNA is about 1:1 by weight.

76. The lipid composition of any one of the preceding claims, wherein the lipid composition is an LNP composition.

77. A method of gene editing, comprising contacting a cell with a lipid composition of any one of the preceding claims.

78. The method of claim 77, wherein the gene editing results in a gene knockout.

79. The method of claim 77, wherein the gene editing results in a gene correction.

80. The method of claim 77, wherein the gene editing results in an insertion.

81. A method of cleaving a DNA, comprising contacting a cell with a lipid composition of any one of claims 1-76.

82. A method of delivering a biologically active agent to a cell, comprising contacting the cell with a lipid composition of any one of claims 1-76.

83. The method of any one of claims 77-82, wherein the contacting step results in a single stranded DNA nick.

84. The method of any one of claims 77-82, wherein the contacting step results in a double-stranded DNA break.

85. The method of any one of claims 77-84, further comprising introducing at least one template nucleic acid into the cell.

86. The method of any one of claims 77-85, wherein the method comprises administering the lipid composition to the cell.

87. The method of any one of claims 77-86, wherein the lipid composition is a first lipid composition, and the method further comprises contacting the cell with a second lipid composition comprising one or more of an mRNA, a gRNA, and a gRNA nucleic acid.

88. The method of claim 87, wherein the second lipid composition is a second lipid composition of any one of claims 1-76.

89. The method of claim 87 or 88, wherein the first and second lipid compositions are administered simultaneously.

90. The method of claim 87 or 88, wherein the first and second lipid compositions are administered sequentially.

91. The method of any one of claims 87-90, wherein the first lipid composition comprises a first gRNA and the second lipid composition comprises a second gRNA, wherein the first and second gRNAs comprise different guide sequences that are complementary to different target sequences.

92. The method of any one of claims 77-91, wherein the cell is a eukaryotic cell.

93. The method of claim 92, wherein the cell is a human cell.

94. The method of any one of claims 77-93, wherein the cell is useful in adoptive cell therapy (ACT).

95. The method of claim 94, wherein the cell is useful in autologous cell therapy.

96. The method of any one of claims 77-95, wherein the cell is a stem cell.

97. The method of claim 96, wherein the stem cell is a hematopoietic stem cell (HSC) or an induced pluripotent stem cell (iPSC).

98. The method of any one of claims 77-97, wherein the cell is an immune cell.

99. The method of claim 98, wherein the immune cell is a leukocyte or a lymphocyte.

100. The method of claim 98, wherein the immune cell is a lymphocyte.

101. The method of claim 100, wherein the lymphocyte is a T cell, a B cell, or an NK cell.

102. The method of claim 100, wherein the lymphocyte is a T cell.

103. The method of claim 100, wherein the lymphocyte is an activated T cell.

104. The method of claim 100, wherein the lymphocyte is a non-activated T cell.

105. The method of any one of claims 77-104, wherein the cell is contacted with the lipid composition in vitro.

106. The method of any one of claims 77-105, wherein the cell is contacted with the lipid composition ex vivo.

107. The method of any one of claims 77-106, wherein the method comprises contacting a tissue of an animal with the lipid.

108. The method of any one of claims 77-107, wherein the method comprises administering the lipid composition to an animal.

109. The method of claim 107 or 108, wherein the animal is a human.

110. The method of any one of claims 77-109, wherein the lipid composition comprises a gRNA targeting a gene that reduces or eliminates surface expression of a T cell receptor, MHC class I, or MHC class II.

111. The method of claim 110, wherein the lipid composition comprises a gRNA targeting TRAC.

112. The method of claim 110, wherein the lipid composition comprises a gRNA targeting TRBC.

113. The method of claim 110, wherein the lipid composition comprises a gRNA targeting CIITA.

114. The method of claim 110, the lipid composition comprises a gRNA targeting HLA-A.

115. The method of claim 110, the lipid composition comprises a gRNA targeting HLA-B.

116. The method of claim 110, the lipid composition comprises a gRNA targeting HLA-C.

117. The method of claim 110, the lipid composition comprises a gRNA targeting B2M.

118. A method of producing multiple genome edits in a cell, comprising
contacting the cell in vitro with at least a first lipid composition of any one of claims 1-76 and a second lipid composition of any one of claims 1-75,
wherein the biologically active agent of the first lipid composition comprises a first guide RNA (gRNA) directed to a first target sequence and optionally a nucleic acid genome editing tool, and
the biologically active agent of the second lipid composition comprises a second gRNA directed to a second target sequence and optionally a nucleic acid genome editing tool thereby producing multiple genome edits in the cell.

119. The method of claim 118, further comprising contacting the cell with a third lipid composition of any one of claims 1-76, wherein the biologically active agent of the third lipid composition comprises a third gRNA directed to a third target sequence and optionally a nucleic acid genome editing tool.

120. The method of claim 119, further comprising contacting the cell with a fourth lipid composition of any one of claims 1-76, wherein the biologically active agent of the fourth lipid composition comprises a fourth gRNA directed to a fourth target sequence and optionally a nucleic acid genome editing tool.

121. The method of claim 120, further comprising contacting the cell with a fifth lipid composition of any one of claims 1-76, wherein the biologically active agent of the fifth lipid composition comprises a fifth gRNA directed to a fifth target sequence and optionally a nucleic acid genome editing tool.

122. The method of claim 121, further comprising contacting the cell with a sixth lipid composition of any one of claims 1-76, wherein the biologically active agent of the sixth lipid composition comprises a sixth gRNA directed to a sixth target sequence and optionally a nucleic acid genome editing tool.

123. The method of any one of claims 118-122, wherein the cell is contacted with at least one lipid composition comprising a genome editing tool.

124. The method of claim 123, wherein the genome editing tool comprises a nucleic acid encoding an RNA-guided DNA binding agent.

125. The method of any one of claims 118-124, wherein the cell is further contacted with a donor nucleic acid for insertion in a target sequence, optionally wherein the donor nucleic acid is provided as a vector.

126. The method of any one of claims 118-125, wherein the lipid compositions are administered sequentially.

127. The method of any one of claims 118-125, wherein at least two lipid compositions are administered simultaneously.

128. The method of any one of claims 118-127, wherein the cell is a eukaryotic cell.

129. The method of claim 128, wherein the cell is a human cell.

130. The method of any one of claims 118-129, wherein the cell is useful in adoptive cell therapy (ACT).

131. The method of claim 130, wherein the cell is useful in autologous cell therapy.

132. The method of any one of claims 118-131, wherein the cell is a stem cell.

133. The method of claim 132, wherein the stem cell is a hematopoietic stem cell (HSC) or an induced pluripotent stem cell (iPSC).

134. The method of any one of claims 118-133, wherein the cell is an immune cell.

135. The method of claim 134, wherein the immune cell is a leukocyte or a lymphocyte.

136. The method of claim 135, wherein the immune cell is a lymphocyte.

137. The method of claim 136, wherein the lymphocyte is a T cell, a B cell, or an NK cell.

138. The method of claim 134, wherein the immune cell is selected from lymphocytes, monocytes, macrophages, mast cells, dendritic cells, granulocytes, primary immune cells, CD3+ cells, CD4+ cells, CD8+ T cells, regulatory T cells (Tregs), B cells, NK cells, and dendritic cells (DC)).

139. The method of claim 134, wherein the cell is a T cell.

140. The method of claim 139, wherein the cell is an activated T cell.

141. The method of claim 139, wherein the cell is a non-activated T cell.

142. The method of claim 125, wherein the cell is a T cell, and the donor nucleic acid comprises regions having homology with corresponding regions of a T cell receptor sequence.

143. The method of any one of claims 118-142, wherein one of the lipid compositions comprises a gRNA targeting TRAC.

144. The method of any one of claims 118-143, wherein one of the lipid compositions comprises a gRNA targeting TRBC.

145. The method of any one of claims 118-144, wherein one of the lipid compositions comprises a gRNA targeting a gene that reduces or eliminates surface expression of MHC class I.

146. The method of any one of claims 118-145, wherein one of the lipid compositions comprises a gRNA targeting a gene that reduces or eliminates surface expression of MHC class II.

147. The method of any one of claims 118-146, wherein one of the lipid compositions comprises a gRNA targeting TRAC, and one of the lipid compositions comprises a gRNA targeting TRBC.

148. The method of any one of claims 118-147, wherein one of the lipid compositions comprises a gRNA targeting TRAC, one of the lipid compositions comprises a gRNA targeting TRBC, one of the lipid compositions comprises a gRNA targeting HLA-A, and one of the lipid compositions comprises a gRNA targeting CIITA.

149. The method of any one of claims 118-147, wherein one of the lipid compositions comprises a gRNA targeting TRAC, one of the lipid compositions comprises a gRNA targeting TRBC, one of the lipid compositions comprises a gRNA targeting HLA-A, and one of the lipid compositions comprises a gRNA targeting a gene that reduces or eliminates surface expression of MHC class II.

150. The method of any one of claims 118-147, wherein one of the lipid compositions comprises a gRNA targeting TRAC, one of the lipid compositions comprises a gRNA targeting TRBC, one of the lipid compositions comprises a gRNA targeting a gene that reduces or eliminates surface expression of MHC class I, and one of the lipid compositions comprises a gRNA targeting CIITA.

151. The method of any one of claims 118-147, wherein one of the lipid compositions comprises a gRNA targeting a gene that reduces or eliminates surface expression of a T cell receptor, one of the lipid compositions comprises a gRNA targeting HLA-A, and one of the lipid compositions comprises a gRNA targeting CIITA.

152. The method of any one of claims 118-147, wherein one of the lipid compositions comprises a gRNA targeting TRAC, one of the lipid compositions comprises a gRNA targeting HLA-A, and one of the lipid compositions comprises a gRNA targeting CIITA.

153. The method of any one of claims 118-147, wherein one of the lipid compositions comprises a gRNA targeting a gene that reduces or eliminates surface expression of a T cell receptor, one of the lipid compositions comprises a gRNA targeting a gene that reduces or eliminates surface expression of MEW class I, and one of the lipid compositions comprises a gRNA targeting CIITA.

154. The method of any one of claims 118-153, further comprising expanding the cells in vitro.

155. A method of producing multiple genome edits in a population of cells, comprising the steps of:
a) contacting the population of cells in vitro with at least a first lipid composition of any one of claims 1-76 and a second lipid composition of any one of claims 1-76,
wherein the biologically active agent of the first lipid composition comprises a first guide RNA (gRNA) directed to a first target sequence and optionally a nucleic acid genome editing tool, and
the biologically active agent of the second lipid composition comprises a second gRNA directed to a second target sequence and optionally a nucleic acid genome editing tool;
thereby producing multiple genome edits in the population of cells.

156. The method of claim 155, further comprising contacting the population of cells with a third lipid composition of any one of claims 1-76, wherein the biologically active agent of the third lipid composition comprises a third gRNA directed to a third target sequence and optionally a nucleic acid genome editing tool.

157. The method of claim 156, further comprising contacting the population of cells with a fourth lipid composition of any one of claims 1-76, wherein the biologically active agent of the fourth lipid composition comprises a fourth gRNA directed to a fourth target sequence and optionally a nucleic acid genome editing tool.

158. The method of claim 157, further comprising contacting the population of cells with a fifth lipid composition of any one of claims 1-76, wherein the biologically active agent of the fifth lipid composition comprises a fifth gRNA directed to a fifth target sequence and optionally a nucleic acid genome editing tool.

159. The method of claim 158, further comprising contacting the population of cells with a sixth lipid composition of any one of claims 1-76, wherein the biologically active agent of the sixth lipid composition comprises a sixth gRNA directed to a sixth target sequence and optionally a nucleic acid genome editing tool.

160. The method of any one of claims 155-159, wherein the population of cells is contacted with at least one lipid composition comprising a genome editing tool.

161. The method of claim 160, wherein the genome editing tool comprises a nucleic acid encoding an RNA-guided DNA binding agent.

162. The method of any one of claims 155-161, wherein the population of cells is further contacted with a donor nucleic acid for insertion in a target sequence, optionally wherein the donor nucleic acid is provided as a vector.

163. The method of any one of claims 155-162, wherein the lipid compositions are administered sequentially.

164. The method of any one of claims 155-162, wherein at least two lipid compositions are administered simultaneously.

165. The method of any one of claims 155-164, wherein the population of cells is a population of eukaryotic cells.

166. The method of claim 166, wherein the population of cells is a population of human cells.

167. The method of any one of claims 155-166, wherein the population of cells is useful in adoptive cell therapy (ACT).

168. The method of claim 167, wherein the population of cells is useful in autologous cell therapy.

169. The method of any one of claims 155-168, wherein the population of cells is a population of stem cells.

170. The method of claim 169, wherein the population of stem cells is a population of hematopoietic stem cells (HSCs) or a population of induced pluripotent stem cells (iPSCs).

171. The method of any one of claims 155-170, wherein the population of cells is a population of immune cells.

172. The method of claim 171, wherein the population of immune cells is a population of leukocytes or a population of lymphocytes.

173. The method of claim 172, wherein the population of immune cells is a population of lymphocytes.

174. The method of claim 173, wherein the population of lymphocytes is a population of T cells, a population of B cells, or a population of NK cells.

175. The method of claim 171, wherein the immune cells are selected from lymphocytes, monocytes, macrophages, mast cells, dendritic cells, granulocytes, primary immune cells, CD3+ cells, CD4+ cells, CD8+ T cells, regulatory T cells (Tregs), B cells, NK cells, and dendritic cells (DC)).

176. The method of claim 171, wherein the population of cells is a population of T cells.

177. The method of claim 176, wherein the cells are activated T cells.

178. The method of claim 176, wherein the cells are non-activated T cells.

179. The method of claim 162, wherein the population of cells is a population of T cells, and the donor nucleic acid comprises regions having homology with corresponding regions of a T cell receptor sequence.

180. The method of any one of claims 155-179, wherein one of the lipid compositions comprises a gRNA targeting TRAC.

181. The method of any one of claims 155-180, wherein one of the lipid compositions comprises a gRNA targeting TRBC.

182. The method of any one of claims 155-181, wherein one of the lipid compositions comprises a gRNA targeting a gene that reduces or eliminates surface expression of MHC class I.

183. The method of any one of claims 155-182, wherein one of the lipid compositions comprises a gRNA targeting a gene that reduces or eliminates surface expression of MHC class II.

184. The method of any one of claims 155-183, wherein one of the lipid compositions comprises a gRNA targeting TRAC, and one of the lipid compositions comprises a gRNA targeting TRBC.

185. The method of any one of claims 155-184, wherein one of the lipid compositions comprises a gRNA targeting TRAC, one of the lipid compositions comprises a gRNA targeting TRBC, one of the lipid compositions comprises a gRNA targeting HLA-A, and one of the lipid compositions comprises a gRNA targeting CIITA.

186. The method of any one of claims 155-184, wherein one of the lipid compositions comprises a gRNA targeting TRAC, one of the lipid compositions comprises a gRNA targeting TRBC, one of the lipid compositions comprises a gRNA targeting HLA-A, and one of the lipid compositions comprises a gRNA targeting a gene that reduces or eliminates surface expression of MHC class II.

187. The method of any one of claims 155-184, wherein one of the lipid compositions comprises a gRNA targeting TRAC, one of the lipid compositions comprises a gRNA targeting TRBC, one of the lipid compositions comprises a gRNA targeting a gene that reduces or eliminates surface expression of MHC class I, and one of the lipid compositions comprises a gRNA targeting CIITA.

188. The method of any one of claims 155-184, wherein one of the lipid compositions comprises a gRNA targeting a gene that reduces or eliminates surface expression of a T cell receptor, one of the lipid compositions comprises a gRNA targeting HLA-A, and one of the lipid compositions comprises a gRNA targeting CIITA.

189. The method of any one of claims 155-184, wherein one of the lipid compositions comprises a gRNA targeting TRAC, one of the lipid compositions comprises a gRNA targeting HLA-A, and one of the lipid compositions comprises a gRNA targeting CIITA.

190. The method of any one of claims 155-184, wherein one of the lipid compositions comprises a gRNA targeting a gene that reduces or eliminates surface expression of a T cell receptor, one of the lipid compositions comprises a gRNA targeting a gene that reduces or eliminates surface expression of MHC class I, and one of the lipid compositions comprises a gRNA targeting CIITA.

191. The method of any one of claims 155-190, further comprising expanding the population of cells in vitro.
